SEQUENCE LISTING (104 sequences)

SEQ ID NO: 1 - protein, 217 aa, Artificial Sequence
  Synthetic polymer.
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, Artificial Sequence
  Synthetic polymer.
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, Artificial Sequence
  Synthetic polymer.
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 11 aa, Artificial Sequence
  Synthetic polymer.
SKYGPPCPSCP

SEQ ID NO: 5 - protein, 11 aa, Artificial Sequence
  Synthetic polymer.
SKYGPPCPPCP

SEQ ID NO: 6 - protein, 6 aa, Artificial Sequence
  Synthetic polymer.
SKYGPP

SEQ ID NO: 7 - protein, 6 aa, Artificial Sequence
  Synthetic polymer.
IEGRMD

SEQ ID NO: 8 - protein, 9 aa, Artificial Sequence
  Synthetic polymer.
GGGVPRDCG

SEQ ID NO: 9 - protein, 15 aa, Artificial Sequence
  Synthetic polymer.
IEGRMDGGGGAGGGG

SEQ ID NO: 10 - protein, 8 aa, Artificial Sequence
  Synthetic polymer.
GGGSGGGS

SEQ ID NO: 11 - protein, 12 aa, Artificial Sequence
  Synthetic polymer.
GGGSGGGGSGGG

SEQ ID NO: 12 - protein, 14 aa, Artificial Sequence
  Synthetic polymer.
EGKSSGSGSESKST

SEQ ID NO: 13 - protein, 4 aa, Artificial Sequence
  Synthetic polymer.
GGSG

SEQ ID NO: 14 - protein, 12 aa, Artificial Sequence
  Synthetic polymer.
GGSGGGSGGGSG

SEQ ID NO: 15 - protein, 15 aa, Artificial Sequence
  Synthetic polymer.
EAAAKEAAAKEAAAK

SEQ ID NO: 16 - protein, 20 aa, Artificial Sequence
  Synthetic polymer.
EAAAREAAAREAAAREAAAR

SEQ ID NO: 17 - protein, 17 aa, Artificial Sequence
  Synthetic polymer.
GGGGSGGGGSGGGGSAS

SEQ ID NO: 18 - protein, 9 aa, Artificial Sequence
  Synthetic polymer.
GGGGAGGGG

SEQ ID NO: 19 - skipped

SEQ ID NO: 20 - protein, 6 aa, Artificial Sequence
  Synthetic polymer.
GSGSGS

SEQ ID NO: 21 - protein, 10 aa, Artificial Sequence
  Synthetic polymer.
GSGSGSGSGS

SEQ ID NO: 22 - protein, 7 aa, Artificial Sequence
  Synthetic polymer.
GGGGSAS

SEQ ID NO: 23 - protein, 20 aa, Artificial Sequence
  Synthetic polymer.
APAPAPAPAPAPAPAPAPAP

SEQ ID NO: 24 - protein, 4 aa, Artificial Sequence
  Synthetic polymer.
CPPC

SEQ ID NO: 25 - protein, 5 aa, Artificial Sequence
  Synthetic polymer.
GGGGS

SEQ ID NO: 26 - protein, 10 aa, Artificial Sequence
  Synthetic polymer.
GGGGSGGGGS

SEQ ID NO: 27 - protein, 15 aa, Artificial Sequence
  Synthetic polymer.
GGGGSGGGGSGGGGS

SEQ ID NO: 28 - protein, 20 aa, Artificial Sequence
  Synthetic polymer.
GGGGSGGGGSGGGGSGGGGS

SEQ ID NO: 29 - protein, 25 aa, Artificial Sequence
  Synthetic polymer.
GGGGSGGGGSGGGGSGGGGSGGGGS

SEQ ID NO: 30 - protein, 30 aa, Artificial Sequence
  Synthetic polymer.
GGGGSGGGGSGGGGSGGGGSGGGGSGGGGS

SEQ ID NO: 31 - protein, 35 aa, Artificial Sequence
  Synthetic polymer.
GGGGSGGGGSGGGGSGGGGSGGGGSGGGGSGGGGS

SEQ ID NO: 32 - protein, 40 aa, Artificial Sequence
  Synthetic polymer.
GGGGSGGGGSGGGGSGGGGSGGGGSGGGGSGGGGSGGGGS

SEQ ID NO: 33 - protein, 16 aa, Artificial Sequence
  Synthetic polymer.
GGSGGSGGGGSGGGGS

SEQ ID NO: 34 - protein, 8 aa, Artificial Sequence
  Synthetic polymer.
GGGGGGGG

SEQ ID NO: 35 - protein, 6 aa, Artificial Sequence
  Synthetic polymer.
GGGGGG

SEQ ID NO: 36 - protein, 5 aa, Artificial Sequence
  Synthetic polymer.
EAAAK

SEQ ID NO: 37 - protein, 10 aa, Artificial Sequence
  Synthetic polymer.
EAAAKEAAAK

SEQ ID NO: 38 - protein, 15 aa, Artificial Sequence
  Synthetic polymer.
EAAAKEAAAKEAAAK

SEQ ID NO: 39 - protein, 12 aa, Artificial Sequence
  Synthetic polymer.
AEAAAKEAAAKA

SEQ ID NO: 40 - protein, 17 aa, Artificial Sequence
  Synthetic polymer.
AEAAAKEAAAKEAAAKA

SEQ ID NO: 41 - protein, 22 aa, Artificial Sequence
  Synthetic polymer.
AEAAAKEAAAKEAAAKEAAAKA

SEQ ID NO: 42 - protein, 27 aa, Artificial Sequence
  Synthetic polymer.
AEAAAKEAAAKEAAAKEAAAKEAAAKA

SEQ ID NO: 43 - protein, 46 aa, Artificial Sequence
  Synthetic polymer.
AEAAAKEAAAKEAAAKEAAAKALEAEAAAKEAAAKEAAAKEAAAKA

SEQ ID NO: 44 - protein, 5 aa, Artificial Sequence
  Synthetic polymer.
PAPAP

SEQ ID NO: 45 - protein, 18 aa, Artificial Sequence
  Synthetic polymer.
KESGSVSSEQLAQFRSLD

SEQ ID NO: 46 - protein, 12 aa, Artificial Sequence
  Synthetic polymer.
GSAGSAAGSGEF

SEQ ID NO: 47 - protein, 5 aa, Artificial Sequence
  Synthetic polymer.
GGGSE

SEQ ID NO: 48 - protein, 5 aa, Artificial Sequence
  Synthetic polymer.
GSESG

SEQ ID NO: 49 - protein, 5 aa, Artificial Sequence
  Synthetic polymer.
GSEGS

SEQ ID NO: 50 - protein, 35 aa, Artificial Sequence
  Synthetic polymer.
GEGGSGEGSSGEGSSSEGGGSEGGGSEGGGSEGGS

SEQ ID NO: 51 - protein, 234 aa, Artificial Sequence
  Synthetic polymer.
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 52 - protein, 234 aa, Artificial Sequence
  Synthetic polymer.
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 53 - protein, 234 aa, Artificial Sequence
  Synthetic polymer.
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 54 - protein, 234 aa, Artificial Sequence
  Synthetic polymer.
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 55 - protein, 234 aa, Artificial Sequence
  Synthetic polymer.
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 56 - protein, 234 aa, Artificial Sequence
  Synthetic polymer.
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 57 - protein, 347 aa, Artificial Sequence
  Synthetic polymer.
SPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLA

SEQ ID NO: 58 - protein, 160 aa, Artificial Sequence
  Synthetic polymer.
AETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLC

SEQ ID NO: 59 - protein, 741 aa, Artificial Sequence
  Synthetic polymer.
SPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLC

SEQ ID NO: 60 - protein, 175 aa, Artificial Sequence
  Synthetic polymer.
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQ

SEQ ID NO: 61 - protein, 220 aa, Artificial Sequence
  Synthetic polymer.
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 62 - protein, 629 aa, Artificial Sequence
  Synthetic polymer.
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 63 - protein, 305 aa, Artificial Sequence
  Synthetic polymer.
QSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQSKYGPP

SEQ ID NO: 64 - protein, 70 aa, Artificial Sequence
  Synthetic polymer.
ASNFDCCLGYTDRILHPKFIVGFTRQLANEGCDINAIIFHTKKKLSVCANPKQTWVKYIVRLLSKKVKNM

SEQ ID NO: 65 - protein, 603 aa, Artificial Sequence
  Synthetic polymer.
QSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDASNFDCCLGYTDRILHPKFIVGFTRQLANEGCDINAIIFHTKKKLSVCANPKQTWVKYIVRLLSKKVKNM

SEQ ID NO: 66 - protein, 127 aa, Artificial Sequence
  Synthetic polymer.
QGVFEDCCLAYHYPIGWAVLRRAWTYRIQEVSGSCNLPAAIFYLPKRHRKVCGNPKSREVQRAMKLLDARNKVFAKLHHNTQTFQAGPHAVKKLSSGNSKLSSSKFSNPISSSKRNVSLLISANSGL

SEQ ID NO: 67 - protein, 660 aa, Artificial Sequence
  Synthetic polymer.
QSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDQGVFEDCCLAYHYPIGWAVLRRAWTYRIQEVSGSCNLPAAIFYLPKRHRKVCGNPKSREVQRAMKLLDARNKVFAKLHHNTQTFQAGPHAVKKLSSGNSKLSSSKFSNPISSSKRNVSLLISANSGL

SEQ ID NO: 68 - protein, 753 aa, Artificial Sequence
  Synthetic polymer.
QSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 69 - protein, 680 aa, Artificial Sequence
  Synthetic polymer.
FKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPESKYGPP

SEQ ID NO: 70 - protein, 1128 aa, Artificial Sequence
  Synthetic polymer.
FKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPESKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 71 - protein, 322 aa, Artificial Sequence
  Synthetic polymer.
DGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRSKYGPP

SEQ ID NO: 72 - protein, 710 aa, Artificial Sequence
  Synthetic polymer.
DGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLC

SEQ ID NO: 73 - protein, 175 aa, Artificial Sequence
  Synthetic polymer.
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWVRSPRRGLQEQEELCFHMWGGKGGLCQSSLSKYGPP

SEQ ID NO: 74 - protein, 563 aa, Artificial Sequence
  Synthetic polymer.
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWVRSPRRGLQEQEELCFHMWGGKGGLCQSSLSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLC

SEQ ID NO: 75 - protein, 563 aa, Artificial Sequence
  Synthetic polymer.
AETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLCSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDTPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWVRSPRRGLQEQEELCFHMWGGKGGLCQSSL

SEQ ID NO: 76 - protein, 713 aa, Artificial Sequence
  Synthetic polymer.
ERLPLTPVSLKVSTNSTRQSLHLQWTVHNLPYHQELKMVFQIQISRIETSNVIWVGNYSTTVKWNQVLHWSWESELPLECATHFVRIKSLVDDAKFPEPNFWSNWSSWEEVSVQDSTGQDILFVFPKDKLVEEGTNVTICYVSRNIQNNVSCYLEGKQIHGEQLDPHVTAFNLNSVPFIRNKGTNIYCEASQGNVSEGMKGIVLFVSKVLEEPKDFSCETEDFKTLHCTWDPGTDTALGWSKQPSQSYTLFESFSGEKKLCTHKNWCNWQITQDSQETYNFTLIAENYLRKRSVNILFNLTHRVYLMNPFSVNFENVNATNAIMTWKVHSIRNNFTYLCQIELHGEGKMMQYNVSIKVNGEYFLSELEPATEYMARVRCADASHFWKWSEWSGQNFTTLEAAPSEAPDVWRIVSLEPGNHTVTLFWKPLSKLHANGKILFYNVVVENLDKPSSSELHSIPAPANSTKLILDRCSYQICVIANNSVGASPASVIVISADPENKEVEEERIAGTEGGFSLSWKPQPGDVIGYVVDWCDHTQDVLGDFQWKNVGPNTTSTVISTDAFRPGVRYDFRIYGLSTKRIACLLEKKTGYSQELAPSDNPHVLVDTLTSHSFTLSWKDYSTESQPGFIQGYHVYLKSKARQCHPRFEKAVLSDGSECCKYKIDNPEEKALIVDNLKPESFYEFFITPFTSAGEGPSATFTKVTTPDEHSSM

SEQ ID NO: 77 - protein, 1123 aa, Artificial Sequence
  Synthetic polymer.
ERLPLTPVSLKVSTNSTRQSLHLQWTVHNLPYHQELKMVFQIQISRIETSNVIWVGNYSTTVKWNQVLHWSWESELPLECATHFVRIKSLVDDAKFPEPNFWSNWSSWEEVSVQDSTGQDILFVFPKDKLVEEGTNVTICYVSRNIQNNVSCYLEGKQIHGEQLDPHVTAFNLNSVPFIRNKGTNIYCEASQGNVSEGMKGIVLFVSKVLEEPKDFSCETEDFKTLHCTWDPGTDTALGWSKQPSQSYTLFESFSGEKKLCTHKNWCNWQITQDSQETYNFTLIAENYLRKRSVNILFNLTHRVYLMNPFSVNFENVNATNAIMTWKVHSIRNNFTYLCQIELHGEGKMMQYNVSIKVNGEYFLSELEPATEYMARVRCADASHFWKWSEWSGQNFTTLEAAPSEAPDVWRIVSLEPGNHTVTLFWKPLSKLHANGKILFYNVVVENLDKPSSSELHSIPAPANSTKLILDRCSYQICVIANNSVGASPASVIVISADPENKEVEEERIAGTEGGFSLSWKPQPGDVIGYVVDWCDHTQDVLGDFQWKNVGPNTTSTVISTDAFRPGVRYDFRIYGLSTKRIACLLEKKTGYSQELAPSDNPHVLVDTLTSHSFTLSWKDYSTESQPGFIQGYHVYLKSKARQCHPRFEKAVLSDGSECCKYKIDNPEEKALIVDNLKPESFYEFFITPFTSAGEGPSATFTKVTTPDEHSSMGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGSRAETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLC

SEQ ID NO: 78 - protein, 500 aa, Artificial Sequence
  Synthetic polymer.
ALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFE

SEQ ID NO: 79 - protein, 910 aa, Artificial Sequence
  Synthetic polymer.
ALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSRAETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLC

SEQ ID NO: 80 - protein, 197 aa, Artificial Sequence
  Synthetic polymer.
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 81 - protein, 606 aa, Artificial Sequence
  Synthetic polymer.
AETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLCGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDECGEDGSGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 82 - protein, 209 aa, Artificial Sequence
  Synthetic polymer.
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 83 - protein, 618 aa, Artificial Sequence
  Synthetic polymer.
AETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLCGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKCGKRGSGSRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 84 - protein, 332 aa, Artificial Sequence
  Synthetic polymer.
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIG

SEQ ID NO: 85 - protein, 742 aa, Artificial Sequence
  Synthetic polymer.
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGSRAETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLC

SEQ ID NO: 86 - protein, 522 aa, Artificial Sequence
  Synthetic polymer.
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSD

SEQ ID NO: 87 - protein, 932 aa, Artificial Sequence
  Synthetic polymer.
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSRAETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLC

SEQ ID NO: 88 - protein, 431 aa, Artificial Sequence
  Synthetic polymer.
YNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTR

SEQ ID NO: 89 - protein, 840 aa, Artificial Sequence
  Synthetic polymer.
YNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGSAETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLC

SEQ ID NO: 90 - protein, 474 aa, Artificial Sequence
  Synthetic polymer.
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDALERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLC

SEQ ID NO: 91 - protein, 883 aa, Artificial Sequence
  Synthetic polymer.
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDALERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSAETPTYPWRDAETGERLVCAQCPPGTFVQRPCRRDSPTTCPCPPRHYTQFWNYLERCRYCNVLCGEREEEARACHATHNRACCRTGFFAHAGFCLEHASCPPGAGVIAPGTPSQNTQCPCPPGTFSASSSSSEQCQPHRNCTALGLALNVPGSSSHDTLC

SEQ ID NO: 92 - protein, 128 aa, Artificial Sequence
  Synthetic polymer.
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 93 - protein, 808 aa, Artificial Sequence
  Synthetic polymer.
YNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 94 - protein, 851 aa, Artificial Sequence
  Synthetic polymer.
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDALERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 95 - protein, 160 aa, Artificial Sequence
  Synthetic polymer.
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 96 - protein, 840 aa, Artificial Sequence
  Synthetic polymer.
YNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 97 - protein, 883 aa, Artificial Sequence
  Synthetic polymer.
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDALERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 98 - protein, 877 aa, Artificial Sequence
  Synthetic polymer.
YNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 99 - protein, 932 aa, Artificial Sequence
  Synthetic polymer.
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDALERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 100 - protein, 762 aa, Artificial Sequence
  Synthetic polymer.
DGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 101 - protein, 774 aa, Artificial Sequence
  Synthetic polymer.
DGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 102 - protein, 235 aa, Artificial Sequence
  Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 103 - protein, 112 aa, Artificial Sequence
  Synthetic polymer.
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 104 - protein, 581 aa, Artificial Sequence
  Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

(19) United States
(12) Patent Application Publication
(10) Pub. No.: US 2025/0345406 A1
(43) Pub. Date: Nov. 13, 2025

(54) CHIMERIC PROTEINS IN AUTOIMMUNITY

(71) Applicant: Shattuck Labs, Inc., Austin, TX (US)

(72) Inventors: Taylor SCHREIBER, Austin, TX (US); George FROMM, Austin, TX (US); Suresh DE SILVA, Austin, TX (US); Louis GONZALEZ, Austin, TX (US)

(21) Appl. No.: 18/280,712

(22) PCT Filed: Mar. 8, 2022
(86) PCT No.: PCT/US22/19313

§ 371 Date: Sep. 7, 2023

Related U.S. Application Data
(60) Provisional application No. 63/158,085, filed on Mar. 8, 2021.

(51) Int. Cl. A61K 39/00; A61P 37/06

(52) U.S. Cl. CPC A61K 39/0008; A61P 37/06

(57) ABSTRACT

The present disclosure relates, inter alia, to compositions and methods, including chimeric proteins, and nucleic acids encoding the chimeric proteins having a first domain comprising an extracellular domain of a first transmembrane protein, a first secreted protein, or a first membrane-anchored extracellular protein and a second domain comprising an extracellular domain of a second transmembrane protein, a second secreted protein, or a second membrane-anchored extracellular protein, in which either or both of the first domain and the second domain decreases self-directed immune system activity when bound to its ligand/receptor. Accordingly, the present disclosure find use in the treatment of autoimmune diseases, and particularly, inflammatory bowel diseases.

### PRIORITY

This application claims the benefit of, and priority to, U.S. Provisional Application No. 63/158,085, filed Mar. 8, 2021, the contents of which are hereby incorporated by reference in their entirety.

### TECHNICAL FIELD

The present disclosure relates to, inter alia, compositions and methods, including chimeric proteins that find use in the treatment of disease, such as in immunotherapies for treating inflammatory bowel disease and/or irritable bowel syndrome.

### DESCRIPTION OF THE TEXT FILE SUBMITTED ELECTRONICALLY

This application contains a sequence listing. It has been submitted electronically via EFS-Web as an ASCII text file entitled “SHK-046PC_116981-5046_ST25”. The sequence listing is 264,343 bytes in size, and was prepared on or about Mar. 4, 2022. The sequence listing is hereby incorporated by reference in its entirety.

### BACKGROUND

Classical criteria defining an autoimmune disease include the demonstration of B-cell clones producing polyclonal pathogenic antibodies specific for autoantigens, T-cell clones that are specific for autoantigens and can transfer autoimmune disease, the precise identification of organ-specific autoantigens, and the reproduction of disease states in experimental animal models.

Inflammatory bowel disease (“IBD”) is an over-arching term used to describe disorders that involve chronic inflammation of the digestive tract. Two types of IBD include ulcerative colitis and Crohn's disease. Though not yet fully understood, it is suspected that IBD is caused by an immune system malfunction, where an abnormal immune response causes the immune system to attack cells of the digestive tract. IBD can cause destructive inflammation and permanent harm to the intestines. Irritable bowel syndrome (“IBS”) does not cause inflammation; its symptoms include chronic abdominal pain, constipation alternating with diarrhea, and abdominal bloating. Though still unclear, both autoimmune and immune-mediated phenomena are involved in inflammatory bowel disease. Immune-mediated phenomena include a variety of abnormalities of humoral and cell-mediated immunity, and a generalized enhanced reactivity against intestinal bacterial antigens in both CD and UC. There are currently no known or approved cures for IBD or IBS. See, Wen and Fiocchi, “Inflammatory Bowel Disease: Autoimmune or Immune-mediated Pathogenesis?” Clinical & Developmental Immunology, Vol. 11: 195-204, 2004.

Accordingly, there is an unmet need for autoimmune therapies that effectively treat autoimmune disease, yet minimize risk for infections.

### SUMMARY

In various aspects, the present disclosure provides for compositions and methods that are useful for immunotherapies for treating an autoimmune disease, such as inflammatory bowel disease (“IBD”) and/or irritable bowel syndrome (“IBS”). For instance, the present disclosure, in part, relates to specific chimeric proteins, and nucleic acids encoding the chimeric proteins, comprising two domains where each or both domains decrease self-directed immune system activity when bound to its ligand/receptor. Importantly, each or both domains decrease immune system activity by activating an immune inhibitory signal or inhibiting an immune activating signal. Accordingly, the present chimeric proteins, nucleic acids encoding the chimeric proteins (without limitations, e.g., modified mRNA), compositions, and methods overcome various deficiencies in bi-specific agents directed to treat autoimmunity.

An aspect of the present disclosure is a chimeric protein, or a nucleic acid encoding the chimeric protein, wherein the chimeric protein comprises a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor.

In an aspect, the present disclosure provides a chimeric protein, or a nucleic acid encoding the chimeric protein, wherein the chimeric protein comprises: (a) a first domain comprising a portion of IL11RA that is capable of binding a IL11RA ligand (e.g. IL-11), (b) a second domain comprising a portion of DcR3 that is capable of binding a DcR3 ligand (e.g. TL1A, LIGHT, FasL), and (c) a linker linking the first domain and the second domain and comprising a hinge-CH2-CH3 Fc domain.

In another aspect, the present disclosure provides a chimeric protein, or a nucleic acid encoding the chimeric protein, wherein the chimeric protein comprises: (a) a first domain comprising a portion of DR3 that is capable of binding a DR3 ligand/receptor (e.g. TL1A), (b) a second domain comprising a portion of PD-L1 that is capable of binding PD-1, and (c) a linker linking the first domain and the second domain and comprising a hinge-CH2-CH3 Fc domain.

In yet another aspect, the present disclosure provides a chimeric protein, or a nucleic acid encoding the chimeric protein, wherein the chimeric protein comprises: (a) a first domain comprising a portion of MADCAM that is capable of binding a MADCAM ligand/receptor, (b) a second domain comprising a portion of CCL20 that is capable of binding a CCL20 receptor, and (c) a linker linking the first domain and the second domain and comprising a hinge-CH2-CH3 Fc domain.

An aspect of the present disclosure is a chimeric protein, or a nucleic acid encoding the chimeric protein, wherein the chimeric protein comprises: (a) a first domain comprising a portion of MADCAM that is capable of binding a MADCAM ligand/receptor, (b) a second domain comprising a portion of CCL25 that is capable of binding a CCL25 receptor, and (c) a linker linking the first domain and the second domain and comprising a hinge-CH2-CH3 Fc domain.

In another aspect, the present disclosure provides a chimeric protein, or a nucleic acid encoding the chimeric protein, wherein the chimeric protein comprises: (a) a first domain comprising a portion of MADCAM that is capable of binding a MADCAM ligand/receptor, (b) a second domain comprising a portion of PD-L1 that is capable of binding PD-1, and (c) a linker linking the first domain and the second domain and comprising a hinge-CH2-CH3 Fc domain.

In another aspect, the present disclosure provides a chimeric protein, or a nucleic acid encoding the chimeric protein, wherein the chimeric protein comprises: (a) a first domain comprising a portion of VCAM that is capable of binding a VCAM ligand/receptor, (b) a second domain comprising a portion of PD-L1 that is capable of binding PD-1, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of IL36R that is capable of binding an IL36R ligand, (b) a second domain comprising a portion of DcR3 that is capable of binding a DcR3 ligand, and (c) a linker linking the first domain and the second domain and comprising a hinge-CH2-CH3 Fc domain.

In another aspect, the present disclosure provides a chimeric protein, or a nucleic acid encoding the chimeric protein, wherein the chimeric protein comprises: (a) a first domain comprising a portion of IL18BP that is capable of binding a IL18BP ligand/receptor, (b) a second domain comprising a portion of DcR3 that is capable of binding a DcR3 ligand, and (c) a linker linking the first domain and the second domain and comprising a hinge-CH2-CH3 Fc domain.

In another aspect, the present disclosure provides a chimeric protein, or a nucleic acid encoding the chimeric protein, wherein the chimeric protein comprises: (a) a first domain comprising a portion of DcR3 that is capable of binding a DcR3 ligand, (b) a second domain comprising a portion of IL18BP that is capable of binding a IL18BP ligand/receptor, and (c) a linker linking the first domain and the second domain and comprising a hinge-CH2-CH3 Fc domain.

In yet another aspect, the present disclosure provides a chimeric protein, or a nucleic acid encoding the chimeric protein, wherein the chimeric protein comprises: (a) a first domain comprising a portion of OSMR that is capable of binding an OSMR ligand, (b) a second domain comprising a portion of DcR3 that is capable of binding a DcR3 ligand, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of gp130 that is capable of binding a gp130 ligand/receptor, (b) a second domain comprising a portion of DcR3 that is capable of binding a DcR3 ligand, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of DcR3 that is capable of binding a DcR3 ligand, (b) a second domain comprising a portion of IL12A that is capable of binding a IL12A ligand/receptor, and (c) a linker linking the first domain and the second domain and comprising a hinge-CH2-CH3 Fc domain.

In another aspect, the present disclosure provides a chimeric protein, or a nucleic acid encoding the chimeric protein, wherein the chimeric protein comprises: (a) a first domain comprising a portion of DcR3 that is capable of binding a DcR3 ligand, (b) a second domain comprising a portion of IL27B that is capable of binding a IL27B ligand/receptor, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of IL23R that is capable of binding an IL23R ligand/receptor, (b) a second domain comprising a portion of DcR3 that is capable of binding a DcR3 ligand, and (c) a linker linking the first domain and the second domain and comprising a hinge-CH2-CH3 Fc domain.

In yet another aspect, the present disclosure provides a chimeric protein, or a nucleic acid encoding the chimeric protein, wherein the chimeric protein comprises: (a) a first domain comprising a portion of IL12RB1 that is capable of binding an IL12RB1 ligand/receptor, (b) a second domain comprising a portion of DcR3 that is capable of binding a DcR3 ligand, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of ITGA4 that is capable of binding an ITGA4 ligand/receptor, (b) a second domain comprising a portion of DcR3 that is capable of binding a DcR3 ligand, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of ITGB7 that is capable of binding an ITGB7 ligand/receptor, (b) a second domain comprising a portion of DcR3 that is capable of binding a DcR3 ligand, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of ITGA4 that is capable of binding an ITGA4 ligand/receptor, (b) a second domain comprising a portion of GITRL that is capable of binding a GITRL receptor, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of ITGB7 that is capable of binding an ITGB7 ligand/receptor, (b) a second domain comprising a portion of GITRL that is capable of binding a GITRL ligand, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of ITGA4 that is capable of binding an ITGA4ligand/receptor, (b) a second domain comprising a portion of IL10 that is capable of binding an IL10 receptor, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of ITGB7 that is capable of binding an ITGB7 ligand/receptor, (b) a second domain comprising a portion of IL10 that is capable of binding an IL10 receptor, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of ITGA4 that is capable of binding an ITGA4 ligand/receptor, (b) a second domain comprising a portion of IL12A that is capable of binding an IL12A ligand/receptor, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of ITGB7 that is capable of binding an ITGB7 ligand/receptor, (b) a second domain comprising a portion of IL27B that is capable of binding an IL27B ligand/receptor, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of IL36R that is capable of binding an IL36R ligand, (b) a second domain comprising a portion of IL12A that is capable of binding an IL12A ligand/receptor, and (c) a linker linking the first domain and the second domain and comprising a hinge-CH2-CH3 Fc domain.

In an aspect, the present disclosure provides a chimeric protein, or a nucleic acid encoding the chimeric protein, wherein the chimeric protein comprises: (a) a first domain comprising a portion of IL36R that is capable of binding an IL36R ligand, (b) a second domain comprising a portion of IL27B that is capable of binding an IL27B ligand/receptor, and (c) a linker linking the first domain and the second domain and comprising a hinge-CH2-CH3 Fc domain.

Another aspect of the present disclosure is a chimeric protein, or a nucleic acid encoding the chimeric protein, wherein the chimeric protein comprises: (a) a first domain comprising a portion of TNFR2 that is capable of binding a TNFR2 ligand/receptor, (b) a second domain comprising a portion of TGF-beta that is capable of binding a TGF-beta ligand/receptor, and (c) a linker linking the first domain and the second domain and comprising a hinge-CH2-CH3 Fc domain.

The chimeric protein of any of the above aspects or embodiments may be a recombinant fusion protein.

The chimeric protein of any of the above aspects or embodiments may be used as a medicament in the treatment of an autoimmune disease, e.g., selected from inflammatory bowel diseases (e.g., ulcerative colitis and Crohn's disease), irritable bowel syndrome (e.g., IBS-C, IBS-D, and IBS-M), ankylosing spondylitis, type 1 diabetes, Grave's disease, Hashimoto's thyroiditis, hypersensitivity reactions (e.g., allergies, hay fever, asthma, and acute edema cause type I hypersensitivity reactions), multiple sclerosis, psoriasis, Addison's disease, rheumatoid arthritis, sarcoidosis, Sjögren's syndrome, systemic lupus erythematosus, and vasculitis.

The present disclosure includes the use of the chimeric protein of any of the above aspects or embodiments in the manufacture of a medicament.

An aspect of the present disclosure is an expression vector comprising a nucleic acid encoding the chimeric protein of any of the above aspects or embodiments.

Another aspect of the present disclosure is a host cell comprising the expression vector of the preceding aspect.

Yet another aspect of the present disclosure is a pharmaceutical composition comprising the chimeric protein, or nucleic acid encoding the chimeric protein of any of the herein disclosed aspects or embodiments.

An aspect of the present disclosure is a method of treating an autoimmune disease comprising administering to a subject in need thereof an effective amount of a pharmaceutical composition comprising the chimeric protein, or nucleic acid encoding the chimeric protein of any of the herein disclosed aspects or embodiments.

Any aspect or embodiment disclosed herein can be combined with any other aspect or embodiment as disclosed herein.

### BRIEF DESCRIPTION OF THE DRAWINGS

FIG. 1A to FIG. 1C show schematic illustrations of proteins that may be used in chimeric proteins of the present disclosure. FIG. 1A shows a Type I transmembrane protein (left protein) and Type II transmembrane protein (right proteins); these proteins differ in that Type I proteins have their amino terminus (“N-”), which comprises its ligand/receptor binding site, directed extracellularly whereas Type II proteins have their carboxy terminus (“C-”), which comprises its ligand/receptor binding site, directed extracellularly. FIG. 1B shows two membrane-anchored extracellular proteins; the illustrated proteins have a ligand/receptor binding site at its amino terminus (“N-”) and is membrane anchored via its carboxy terminus (left protein) or have a ligand/receptor binding site at its carboxy terminus (“C-”) and is membrane anchored via its amino terminus (right protein); however, membrane-anchored extracellular proteins may be membrane anchored via other locations along the protein's amino acid sequence. FIG. 1C shows two secreted proteins (which lack a transmembrane domain or a membrane anchorage); the left protein has its ligand/receptor binding site at it amino terminus (“N-”) and the right protein has its ligand/receptor binding site at its carboxy terminus (“C-”).

FIG. 2A to FIG. 2D show schematic illustrations of chimeric proteins of the present disclosure. FIG. 2A shows a chimeric protein comprising a first domain with a ligand/receptor binding site at its amino terminus and a second domain with a ligand/receptor binding site at its carboxy terminus. Non-limiting examples of this configuration of chimeric protein include a chimeric protein comprising a portion of a Type I transmembrane protein as its first domain and a portion of a Type II transmembrane protein as its second domain and a chimeric protein comprising a portion of a Type I transmembrane protein as its first domain and a portion of a secreted protein as its second domain. FIG. 2B shows a chimeric protein comprising a first domain with a ligand/receptor binding site at its amino terminus and a second domain with a ligand/receptor binding site at its amino terminus. Non-limiting examples of this configuration of chimeric protein include a chimeric protein comprising a portion of a Type I transmembrane protein as its first domain and a portion of a Type I transmembrane protein as its second domain and a chimeric protein comprising a portion of a Type I transmembrane protein as its first domain and a portion of a secreted protein as its second domain. FIG. 2C shows a chimeric protein comprising a first domain with a ligand/receptor binding site at its carboxy terminus and a second domain with a ligand/receptor binding site at its carboxy terminus. Non-limiting examples of this configuration of chimeric protein include a chimeric protein comprising a portion of a membrane anchored protein as its first domain and a portion of secreted protein as its second domain and a chimeric protein comprising a portion of secreted protein as its first domain and a portion of a Type II transmembrane protein as its second domain. FIG. 2D shows a chimeric protein comprising a first domain with a ligand/receptor binding site at its carboxy terminus and a second domain with a ligand/receptor binding site at its amino terminus. Non-limiting examples of this configuration of chimeric protein include a chimeric protein comprising a portion of secreted protein as its first domain and a portion of a membrane anchored protein as its second domain and a chimeric protein comprising a portion of Type II transmembrane protein as its first domain and a portion of a Type I transmembrane protein as its second domain.

FIG. 3 depicts an overall schematic of the experiment. Specifically, mice were given 3% DSS ad libitum starting at Day 0, and the DSS withdrawn on Day 7. Concurrently, the mice were administered the following (according to the group division above) on Days 0, 3, and 5: (1) No DSS (control); (2) DSS only; (3) mCTLA-4 Ig (control); (4) control chimeric protein A; (5) mTNFR2-Fc-TGF-beta chimeric protein; and (6) control chimeric protein B. The mice were weighed daily, with an endpoint if the weight loss was greater than 20%. On Day 14, the mice were weighed for a final time and sacrificed.

FIG. 4 shows mouse weight (g) with 3% DSS and various treatments over the course of the two-week experiment. The results shown in FIG. 4 demonstrate that, among the chimeric protein treatments, the group that was administered mTNFR2-Fc-TGF-beta (mTNFR2-Fc-TGF-beta) exhibited the greatest protection from weight loss.

FIG. 5 shows that the mice administered with mTNFR2-Fc-TGF-beta chimeric protein suffered the least from a percent change from their original weight.

### DETAILED DESCRIPTION

The present disclosure is based, in part, on the discovery that chimeric proteins can be engineered from a first domain comprising an extracellular domain of a first transmembrane protein, a first secreted protein, or a first membrane-anchored extracellular protein and a second domain comprising an extracellular domain of a second transmembrane protein, a second secreted protein, or a second membrane-anchored extracellular protein. In these chimeric proteins, either or both of the first domain and the second domain decreases self-directed immune system activity when bound to its ligand/receptor. Accordingly, the present disclosure finds use in the treatment of an autoimmune disease, which occurs when a subject's own antigens become targets for an immune response.

The present chimeric proteins provide advantages including, without limitation, ease of use and ease of production. This is because two distinct immunotherapy agents are combined into a single product which may allow for a single manufacturing process instead of two independent manufacturing processes. In addition, administration of a single agent instead of two separate agents allows for easier administration and greater patient compliance. Further, in contrast to, for example, monoclonal antibodies, which are large multimeric proteins containing numerous disulfide bonds and post-translational modifications such as glycosylation, the present chimeric proteins are easier and more cost effective to manufacture.

Importantly, since a chimeric protein of the present disclosure comprises two ligand/receptor binding domains, it is capable of, via two cellular pathways, decreasing immune system activity by activating an immune inhibitory signal and/or by inhibiting an immune activating signal. This dual-action is more likely to provide any anti-autoimmune effect in a subject. Moreover, since the chimeric proteins and methods using the chimeric proteins operate by multiple distinct pathways, they can be efficacious, at least, in patients who do not respond, respond poorly, or become resistant to treatments that target one of the pathways. Thus, a patient who is a poor responder to treatments acting via one of the two pathways, can receive a therapeutic benefit by targeting multiple pathways.

### Chimeric Proteins

An aspect of the present disclosure is a chimeric protein of a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor.

In embodiments, the portion of the first domain is capable of binding the native ligand/receptor for the transmembrane protein, the secreted protein, or the membrane-anchored extracellular protein.

In embodiments, the portion of the second domain is capable of binding the native ligand/receptor for the transmembrane protein, the secreted protein, or the membrane-anchored extracellular protein.

In embodiments, the first domain comprises substantially the entire extracellular domain of the transmembrane protein, substantially the entire secreted protein, or substantially the entire membrane-anchored extracellular protein.

In embodiments, the second domain comprises substantially the entire extracellular domain of the transmembrane protein, substantially the entire secreted protein, or substantially the entire membrane-anchored extracellular protein.

In embodiments, the binding of the portion of the first domain to its ligand/receptor decreases immune system activity by activating an immune inhibitory signal or inhibiting an immune activating signal.

In embodiments, the binding of the portion of the second domain to its ligand/receptor decreases immune system activity by activating an immune inhibitory signal or by inhibiting an immune activating signal.

In embodiments, the portion of the first domain comprises a transmembrane protein, a secreted protein, or a membrane-anchored extracellular protein selected from TNFR2, IL11RA, DR3, MADCAM, VCAM, IL36R, IL18BP, DcR3, OSMR, gp130, IL23R, IL12RB1, ITGA4, and ITGB7.

In embodiments, the portion of the second domain comprises a transmembrane protein, a secreted protein, or a membrane-anchored extracellular protein selected from TGF-beta, DcR3, PD-L1, CCL20, CCL25, IL18BP, IL12A, IL27B, GITRL, and IL10.

In embodiments, the first domain comprises a portion of IL11RA and the second domain comprises a portion of DcR3.

In embodiments, the first domain comprises a portion of DR3 and the second domain comprises a portion of PD-L1.

In embodiments, the first domain comprises a portion of MADCAM and the second domain comprises a portion of CCL20.

In embodiments, the first domain comprises a portion of MADCAM and the second domain comprises a portion of CCL25.

In embodiments, the first domain comprises a portion of MADCAM and the second domain comprises a portion of PD-L1.

In embodiments, the first domain comprises a portion of VCAM and the second domain comprises a portion of PD-L1.

In embodiments, the first domain comprises a portion of IL36R and the second domain comprises a portion of DcR3.

In embodiments, the first domain comprises a portion of IL18BP and the second domain comprises a portion of DcR3.

In embodiments, the first domain comprises a portion of DcR3 and the second domain comprises a portion of IL18BP.

In embodiments, the first domain comprises a portion of OSMR and the second domain comprises a portion of DcR3.

In embodiments, the first domain comprises a portion of gp130 and the second domain comprises a portion of DcR3.

In embodiments, the first domain comprises a portion of DcR3 and the second domain comprises a portion of IL12A.

In embodiments, the first domain comprises a portion of DcR3 and the second domain comprises a portion of IL27B.

In embodiments, the first domain comprises a portion of IL23R and the second domain comprises a portion of DcR3.

In embodiments, the first domain comprises a portion of IL12RB1 and the second domain comprises a portion of DcR3.

In embodiments, the first domain comprises a portion of ITGA4 and the second domain comprises a portion of DcR3.

In embodiments, the first domain comprises a portion of ITGB7 and the second domain comprises a portion of DcR3.

In embodiments, the first domain comprises a portion of ITGA4 and the second domain comprises a portion of GITRL.

In embodiments, the first domain comprises a portion of ITGB7 and the second domain comprises a portion of GITRL.

In embodiments, the first domain comprises a portion of ITGA4 and the second domain comprises a portion of IL10.

In embodiments, the first domain comprises a portion of ITGB7 and the second domain comprises a portion of IL10.

In embodiments, the first domain comprises a portion of ITGA4 and the second domain comprises a portion of IL12A.

In embodiments, the first domain comprises a portion of ITGB7 and the second domain comprises a portion of IL27B.

In embodiments, the first domain comprises a portion of IL36R and the second domain comprises a portion of IL12A.

In embodiments, the first domain comprises a portion of IL36R and the second domain comprises a portion of IL27B.

In embodiments, the first domain comprises a portion of TNFR2 and the second domain comprises an extracellular domain of a transmembrane protein selected from TGF-beta, 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL; in embodiments, the second domain comprises TGF-beta. In embodiments, the CLEC family member is selected from AICL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+DC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D.

In embodiments, the binding of either or both of the first domain and the second domains to its ligand/receptor occurs with slow off rates (Kott), which provides along interaction of a receptor and its ligand. In embodiments, the long interaction provides a prolonged decrease in immune system activity which comprises sustained activation of an immune inhibitory signal and/or a sustained inhibition of an immune activating signal. In embodiments, the sustained activation of the immune inhibitory signal and/or the sustained inhibition of the immune activating signal reduces the activity or proliferation of an immune cell, e.g., a B cell or a T cell. In embodiments, the sustained activation of the immune inhibitory signal and/or the sustained inhibition of the immune activating signal decreases synthesis and/or decreases release of a pro-inflammatory cytokine. In embodiments, the sustained activation of the immune inhibitory signal and/or the sustained inhibition of the immune activating signal increases synthesis and/or increases release of an anti-inflammatory cytokine. In embodiments, the sustained activation of the immune inhibitory signal and/or the sustained inhibition of the immune activating signal decreases antibody production and/or decreases secretion of antibodies by a B cell, e.g., an antibody that recognizes a self-antigen. In embodiments, the sustained activation of the immune inhibitorysignal and/or the sustained inhibition of the immune activating signal decreases the activity of and/or decreases the number of T cytotoxic cells, e.g., which recognize a self-antigen and kill cells presenting or expressing the self-antigen. In embodiments, the sustained activation of the immune inhibitory signal and/or the sustained inhibition of the immune activating signal increases the activity and/or increases the number of T regulatory cells.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain, e.g., a hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In an aspect, the present disclosure provides a chimeric protein, or a nucleic acid encoding the chimeric protein, wherein the chimeric protein comprises: (a) a first domain comprising a portion of I111RA that is capable of binding a IL11RA ligand, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL11RA-Fc-DcR3.

In another aspect, the present disclosure provides a chimeric protein, or a nucleic acid encoding the chimeric protein, wherein the chimeric protein comprises: (a) a first domain comprising a portion of DR3 that is capable of binding a DR3 ligand/receptor, (b) a second domain comprising a portion of PD-L1 that is capable of binding PD-1, and (c) a linker linking the first domain and the second domain and comprising a hinge-CH2-CH3 Fc domain. In some embodiments, this chimeric protein is referred to herein as DR3-Fc-PD-L1.

In yet another aspect, the present disclosure provides a chimeric protein, or a nucleic acid encoding the chimeric protein, wherein the chimeric protein comprises: (a) a first domain comprising a portion of MADCAM that is capable of binding a MADCAM ligand/receptor, (b) a second domain comprising a portion of CCL20 that is capable of binding a CCL20 receptor, and (c) a linker linking the first domain and the second domain and comprising a hinge-CH2-CH3 Fc domain. In some embodiments, this chimeric protein is referred to herein as MADCAM-Fc-CCL20.

An aspect of the present disclosure is a chimeric protein, or a nucleic acid encoding the chimeric protein, wherein the chimeric protein comprises: (a) a first domain comprising a portion of MADCAM that is capable of binding a MADCAM ligand/receptor, (b) a second domain comprising a portion of CCL25 that is capable of binding a CCL25 receptor, and (c) a linker linking the first domain and the second domain and comprising a hinge-CH2-CH3 Fc domain. In some embodiments, this chimeric protein is referred to herein as MADCAM-Fc-CCL25.

In another aspect, the present disclosure provides a chimeric protein, or a nucleic acid encoding the chimeric protein, wherein the chimeric protein comprises: (a) a first domain comprising a portion of MADCAM that is capable of binding a MADCAM ligand/receptor, (b) a second domain comprising a portion of PD-L1 that is capable of binding PD-1, and (c) a linker linking the first domain and the second domain and comprising a hinge-CH2-CH3 Fc domain. In some embodiments, this chimeric protein is referred to herein as MADCAM-Fc-PD-1.

In another aspect, the present disclosure provides a chimeric protein, or a nucleic acid encoding the chimeric protein, wherein the chimeric protein comprises: (a) a first domain comprising a portion of VCAM that is capable of binding a VCAM ligand/receptor, (b) a second domain comprising a portion of PD-L1 that is capable of binding PD-1, and (c) a linker linking the first domain and the second domain and comprising a hinge-CH2-CH3 Fc domain. In some embodiments, this chimeric protein is referred to herein as VCAM-Fc-PD-1.

In an aspect, the present disclosure provides a chimeric protein, or a nucleic acid encoding the chimeric protein, wherein the chimeric protein comprises: (a) a first domain comprising a portion of IL36R that is capable of binding an IL36R ligand, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL36R-Fc-DcR3.

In another aspect, the present disclosure provides a chimeric protein, or a nucleic acid encoding the chimeric protein, wherein the chimeric protein comprises: (a) a first domain comprising a portion of IL18BP that is capable of binding a IL18BP ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL18BP-Fc-DcR3.

In another aspect, the present disclosure provides a chimeric protein, or a nucleic acid encoding the chimeric protein, wherein the chimeric protein comprises: (a) a first domain comprising a portion of DcR3 that is capable of binding a DcR3 ligand, (b) a second domain comprising a portion of IL18BP that is capable of binding a IL18BP ligand/receptor, and (c) a linker linking the first domain and the second domain and comprising a hinge-CH2-CH3 Fc domain. In some embodiments, this chimeric protein is referred to herein as DcR3-Fc-IL18BP.

In yet another aspect, the present disclosure provides a chimeric protein, or a nucleic acid encoding the chimeric protein, wherein the chimeric protein comprises: (a) a first domain comprising a portion of OSMR that is capable of binding an OSMR ligand, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as OSMR-Alpha-DcR3.

In an aspect, the present disclosure provides a chimeric protein, or a nucleic acid encoding the chimeric protein, wherein the chimeric protein comprises: (a) a first domain comprising a portion of gp130 that is capable of binding a gp130 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as gp130-Beta-DcR3.

In an aspect, the present disclosure provides a chimeric protein, or a nucleic acid encoding the chimeric protein, wherein the chimeric protein comprises: (a) a first domain comprising a portion of DcR3 that is capable of binding a DcR3 ligand, (b) a second domain comprising a portion of IL12A that is capable of binding a IL12A ligand/receptor, and (c) a linker linking the first domain and the second domain and comprising a hinge-CH2-CH3 Fc domain. In some embodiments, this chimeric protein is referred to herein as DcR3-Alpha-IL12A.

In another aspect, the present disclosure provides a chimeric protein, or a nucleic acid encoding the chimeric protein, wherein the chimeric protein comprises: (a) a first domain comprising a portion of DcR3 that is capable of binding a DcR3 ligand, (b) a second domain comprising a portion of IL27B that is capable of binding a IL27B ligand/receptor, and (c) a linker linking the first domain and the second domain and comprising a hinge-CH2-CH3 Fc domain. In some embodiments, this chimeric protein is referred to herein as DcR3-Beta-IL27B.

In an aspect, the present disclosure provides a chimeric protein, or a nucleic acid encoding the chimeric protein, wherein the chimeric protein comprises: (a) a first domain comprising a portion of IL23R that is capable of binding an IL23R ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL23R-Alpha-DcR3.

In yet another aspect, the present disclosure provides a chimeric protein, or a nucleic acid encoding the chimeric protein, wherein the chimeric protein comprises: (a) a first domain comprising a portion of IL12RB1 that is capable of binding an IL12RB1 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL12RB1-Beta-DcR3.

In an aspect, the present disclosure provides a chimeric protein, or a nucleic acid encoding the chimeric protein, wherein the chimeric protein comprises: (a) a first domain comprising a portion of ITGA4 that is capable of binding an ITGA4 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as ITGA4-Alpha-DcR3.

In an aspect, the present disclosure provides a chimeric protein, or a nucleic acid encoding the chimeric protein, wherein the chimeric protein comprises: (a) a first domain comprising a portion of ITGB7 that is capable of binding an ITGB7 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as ITGB7-Beta-DcR3.

In an aspect, the present disclosure provides a chimeric protein, or a nucleic acid encoding the chimeric protein, wherein the chimeric protein comprises: (a) a first domain comprising a portion of ITGA4 that is capable of binding an ITGA4 ligand/receptor, (b) a second domain comprising a portion of GITRL that is capable of binding a GITRL ligand, and (c) a linker linking the first domain and the second domain and comprising a hinge-CH2-CH3 Fc domain. In some embodiments, this chimeric protein is referred to herein as ITGA4-Alpha-GITRL.

In an aspect, the present disclosure provides a chimeric protein, or a nucleic acid encoding the chimeric protein, wherein the chimeric protein comprises: (a) a first domain comprising a portion of ITGB7 that is capable of binding an ITGB7 ligand/receptor, (b) a second domain comprising a portion of GITRL that is capable of binding a GITRL ligand, and (c) a linker linking the first domain and the second domain and comprising a hinge-CH2-CH3 Fc domain. In some embodiments, this chimeric protein is referred to herein as ITGB7-Beta-GITRL.

In an aspect, the present disclosure provides a chimeric protein, or a nucleic acid encoding the chimeric protein, wherein the chimeric protein comprises: (a) a first domain comprising a portion of ITGA4 that is capable of binding an ITGA4ligand/receptor, (b) a second domain comprising a portion of IL10 that is capable of binding an IL10 receptor, and (c) a linker linking the first domain and the second domain and comprising a hinge-CH2-CH3 Fc domain. In some embodiments, this chimeric protein is referred to herein as ITGA4-Alpha-IL10.

In an aspect, the present disclosure provides a chimeric protein, or a nucleic acid encoding the chimeric protein, wherein the chimeric protein comprises: (a) a first domain comprising a portion of ITGB7 that is capable of binding an ITGB7 ligand/receptor, (b) a second domain comprising a portion of IL10 that is capable of binding an IL10 receptor, and (c) a linker linking the first domain and the second domain and comprising a hinge-CH2-CH3 Fc domain. In some embodiments, this chimeric protein is referred to herein as ITGB7-Beta-IL10.

In an aspect, the present disclosure provides a chimeric protein, or a nucleic acid encoding the chimeric protein, wherein the chimeric protein comprises: (a) a first domain comprising a portion of ITGA4 that is capable of binding an ITGA4 ligand/receptor, (b) a second domain comprising a portion of IL12A that is capable of binding an IL12A ligand/receptor, and (c) a linker linking the first domain and the second domain and comprising a hinge-CH2-CH3 Fc domain. In some embodiments, this chimeric protein is referred to herein as ITGA4-Alpha-IL12A.

In an aspect, the present disclosure provides a chimeric protein, or a nucleic acid encoding the chimeric protein, wherein the chimeric protein comprises: (a) a first domain comprising a portion of ITGB7 that is capable of binding an ITGB7 ligand/receptor, (b) a second domain comprising a portion of IL27B that is capable of binding an IL27B ligand/receptor, and (c) a linker linking the first domain and the second domain and comprising a hinge-CH2-CH3 Fc domain. In some embodiments, this chimeric protein is referred to herein as ITGB7-Beta-IL27B.

In an aspect, the present disclosure provides a chimeric protein, or a nucleic acid encoding the chimeric protein, wherein the chimeric protein comprises: (a) a first domain comprising a portion of IL36R that is capable of binding an IL36R ligand, (b) a second domain comprising a portion of IL12A that is capable of binding an IL12A ligand/receptor, and (c) a linker linking the first domain and the second domain and comprising a hinge-CH2-CH3 Fc domain. In some embodiments, this chimeric protein is referred to herein as IL36R-Alpha-IL12A.

In an aspect, the present disclosure provides a chimeric protein, or a nucleic acid encoding the chimeric protein, wherein the chimeric protein comprises: (a) a first domain comprising a portion of IL36R that is capable of binding an IL36R ligand, (b) a second domain comprising a portion of IL27B that is capable of binding an IL27B ligand/receptor, and (c) a linker linking the first domain and the second domain and comprising a hinge-CH2-CH3 Fc domain. In some embodiments, this chimeric protein is referred to herein as IL36R-Beta-IL27B.

Another aspect of the present disclosure is a chimeric protein, or a nucleic acid encoding the chimeric protein, wherein the chimeric protein comprises: (a) a first domain comprising a portion of TNFR2 that is capable of binding a TNFR2 ligand/receptor, (b) a second domain comprising a portion of TGF-beta that is capable of binding a TGF-beta ligand/receptor, and (c) a linker linking the first domain and the second domain and comprising a hinge-CH2-CH3 Fc domain. In some embodiments, this chimeric protein is referred to as TNFR2-Fc-TGF-beta.

In embodiments, the hinge-CH2-CH3 Fc domain comprises at least one cysteine residue capable of forming a disulfide bond. In embodiments, the hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In a chimeric protein of the present disclosure, the chimeric protein is a recombinant fusion protein, e.g., a single polypeptide having the extracellular domains disclosed herein. For example, in embodiments, the chimeric protein is translated as a single unit in a prokaryotic cell, a eukaryotic cell, or a cell-free expression system.

In embodiments, the present chimeric protein is producible in a mammalian host cell as a secretable and fully functional single polypeptide chain.

In embodiments, chimeric protein refers to a recombinant protein of multiple polypeptides, e.g., multiple extracellular domains disclosed herein, that are combined (via covalent or no-covalent bonding) to yield a single unit, e.g., in vitro (e.g., with one or more synthetic linkers disclosed herein).

In embodiments, the chimeric protein is chemically synthesized as one polypeptide or each domain may be chemically synthesized separately and then combined. In embodiments, a portion of the chimeric protein is translated and a portion is chemically synthesized.

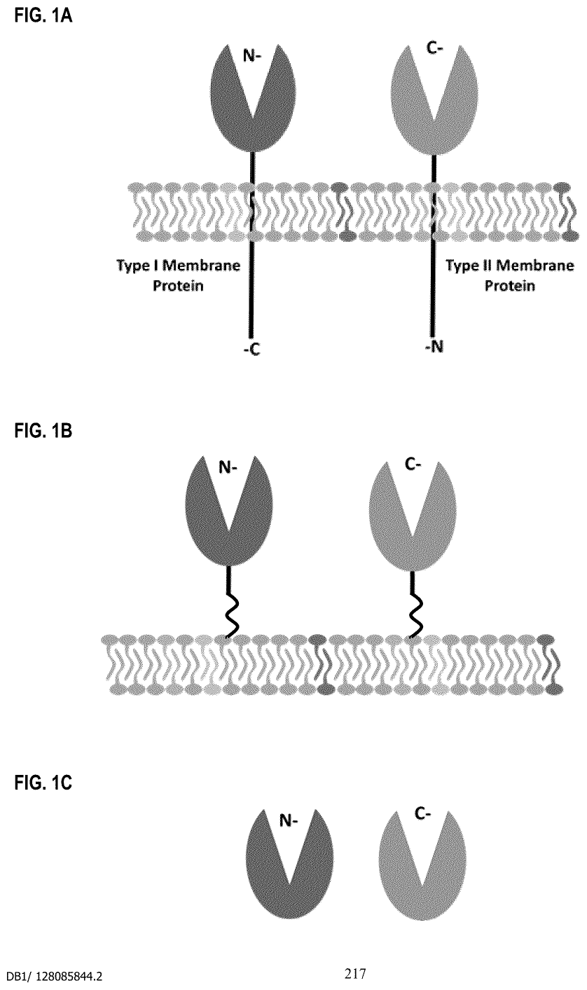

Transmembrane proteins typically consist of an extracellular domain, one or a series of transmembrane domains, and an intracellular domain. Without wishing to be bound by theory, the extracellular domain of a transmembrane protein is responsible for interacting with a soluble receptor or ligand or membrane-bound receptor or ligand (e.g., a membrane of an adjacent cell). Without wishing to be bound by theory, the transmembrane domain(s) is responsible for localizing the transmembrane protein to the plasma membrane. Without wishing to be bound by theory, the intracellular domain of a transmembrane protein is responsible for coordinating interactions with cellular signaling molecules to coordinate intracellular responses with the extracellular environment (or visa-versa). Illustrations of transmembrane proteins are shown in FIG. 1A.

In contrast to transmembrane proteins, membrane-anchored extracellular proteins lack a transmembrane domain that spans, at least part, of a cell's lipid bilayer. Instead, these proteins are associated with the extracellular face of a cell's membrane. The association may be a result of hydrophobic interactions between the bilayer and exposed nonpolar residues at the surface of a protein, by specific non-covalent binding interactions with regulatory lipids, or through their attachment to covalently bound lipid anchors (including the lipids glycosylphosphatidylinositol (GPI) and cholesterol). Alternately, membrane-anchored extracellular proteins may indirectly be associated with the cell's lipid bilayer via another protein that is directly associated with the membrane, including transmembrane proteins. Illustrations of membrane-anchored extracellular proteins are shown in FIG. 1B.

A secreted protein can be defined as a protein which is actively transported out of the cell. Medically important secreted proteins include cytokines, coagulation factors, enzymes, growth factors, hormones, and other signaling molecules. Often secreted proteins have an amino terminal comprising a signal sequence consisting of 6 to 12 amino acids with hydrophobic side chains. The signal sequence, at least, permits packaging of secreted proteins into vesicles which, when fused with the cell's membrane, the secreted protein leaves the cell. Illustrations of secreted proteins are shown in FIG. 1C.

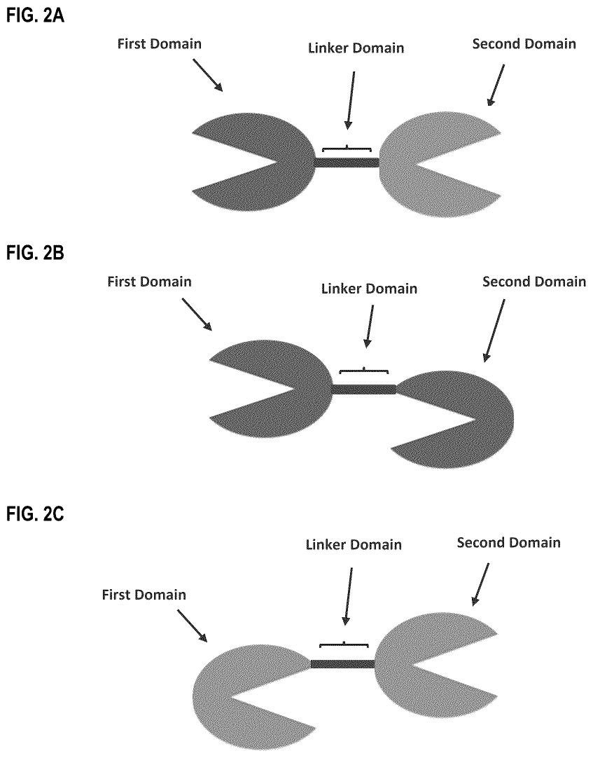

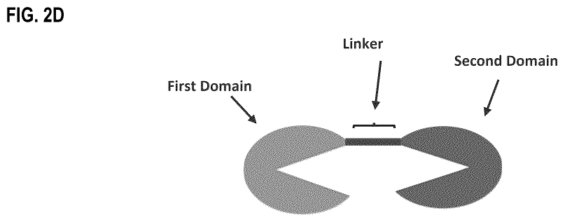

FIG. 2A to FIG. 2D show schematic illustrations of chimeric proteins of the present disclosure. FIG. 2A shows a chimeric protein comprising a first domain with a ligand/receptor binding site at its amino terminus and a second domain with a ligand/receptor binding site at its carboxy terminus. FIG. 2B shows a chimeric protein comprising a first domain with a ligand/receptor binding site at its amino terminus and a second domain with a ligand/receptor binding site at its amino terminus. FIG. 2C shows a chimeric protein comprising a first domain with a ligand/receptor binding site at its carboxy terminus and a second domain with a ligand/receptor binding site at its carboxy terminus. FIG. 2D shows a chimeric protein comprising a first domain with a ligand/receptor binding site at its carboxy terminus and a second domain with a ligand/receptor binding site at its amino terminus.

Chimeric proteins of the present disclosure have a first domain which is sterically capable of binding its ligand/receptor and/or a second domain which is sterically capable of binding its ligand/receptor. This means that there is sufficient overall flexibility in the chimeric protein and/or physical distance between a first domain (or portion thereof) and the rest of the chimeric protein such that the ligand/receptor binding domain of the first domain is not sterically hindered from binding its ligand/receptor and/or there is sufficient physical distance between a second domain (or portion thereof) and the rest of the chimeric protein such that the ligand/receptor binding domain of the second domain is not sterically hindered from binding its ligand/receptor. This flexibility and/or physical distance (which is herein referred to as “slack”) may be normally present in the first and/or second domain(s), normally present in the linker, and/or normally present in the chimeric protein (as a whole). Alternately, or additionally, the chimeric protein may be modified by including one or more additional amino acid sequences (e.g., the joining linkers described below) or synthetic linkers (e.g., a polyethylene glycol (PEG) linker) which provide additional slack needed to avoid steric hindrance. Further description of linkers useful in the present disclosure, and especially the linkers of SEQ ID NO: 1 to SEQ ID NO: 3, are included in the next section of this disclosure entitled “Linkers”.

### IL11RA-Fc-DcR3

In embodiments, the chimeric protein is capable of contemporaneously binding the I111RA ligand and the DcR3 ligand. In embodiments, the I111RA ligand is interleukin-11 (IL-11), and the DcR3 ligand is Fas ligand (FasL), LIGHT, or TL1A. Interleukin 11 receptor alpha (IL11RA) is a member of the hematopoietic cytokine receptor family. IL11RA signals through a common receptor subunit termed glycoprotein 130 (gp130). Binding of I11RA with its ligand induces gp130 homodimerization, which leads to activation of the Janus kinase/STAT signal transduction pathway. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Upregulation of DcR3 during inflammatory reactions exerts negative-feedback to suppress inflammation. Accordingly, a chimeric protein comprising the extracellular domain of IL11RA and the extracellular domain of DcR3 is capable of contemporaneously stimulating an immune activating signal (via IL11RA) and suppressing inflammation by neutralizing pro-inflammatory cytokines (via DcR3). In embodiments, this chimeric protein is referred to herein as IL11RA-Fc-DcR3.

In embodiments, the chimeric proteins of the present disclosure comprise variants of a portion of IL11RA which includes its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the portion of IL11RA, e.g., human IL11RA, which comprises its receptor-binding domain.

In embodiments, the extracellular domain of IL11RA has the following amino acid sequence:

(SEQ ID NO: 57)
SPCPQAWGPPGVQYGQPGRSVKLCCPGVTAGDPVSWFRDGEPKLLQGPDSGLGHELVLAQADS
TDEGTYICQTLDGALGGTVTLQLGYPPARPVVSCQAADYENFSCTWSPSQISGLPTRYLTSYRKKT
VLGADSQRRSPSTGPWPCPQDPLGAARCVVHGAEFWSQYRINVTEVNPLGASTRLLDVSLQSILR
PDPPQGLRVESVPGYPRRLRASWTYPASWPCQPHFLLKFRLQYRPAQHPAWSTVEPAGLEEVITD
AVAGLPHAVRVSARDFLDAGTWSTWSPEAWGTPSTGTIPKEIPAWGQLHTQPEVEPQVDSPAPPR
PSLQPHPRLLDHRDSVEQVAVLA.

In embodiments, a chimeric protein comprises a variant of the portion of IL11RA comprising its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 57.

In embodiments, the first domain of a chimeric protein comprises an amino acid sequence that is at least 95% identical to the amino acid sequence of SEQ ID NO: 57.

One of ordinary skill may select variants of the known amino acid sequence of IL11RA by consulting the literature, e.g., Brischoux-Boucher et al., “IL11RA-related Crouzon-like autosomal recessive craniosynostosis in 10 new patients: Resemblances and differences,” Clin. Genet. 94 (3-4), 373-380 (2018); Jiang et al., “miR23b inhibits proliferation of SMMC7721 cells by directly targeting IL11,” Mol Med Rep 18 (2), 1591-1599 (2018); Lokau et al., “The length of the interleukin-11 receptor stalk determines its capacity for classic signaling,” J. Biol. Chem. 293 (17), 6398-6409 (2018); Barton et al., “Identification of three distinct receptor binding sites of murine interleukin-11,” J. Biol. Chem. 274 (9), 5755-5761 (1999); Schleinkofer et al., “Identification of the domain in the human interleukin-11 receptor that mediates ligand binding.” J Mol Biol. 2001 Feb. 16; 306(2): 263-74; Kurth et al., “Activation of the Signal Transducer Glycoprotein 130 by Both IL-6 and IL-11 Requires Two Distinct Binding Epitopes. “J. Immunology, Vol. 162, Issue 3 (1999); and Keupp et al., “Mutations in the interleukin receptor I11RA cause autosomal recessive Crouzon-like craniosynostosis.” Mol Genet Genomic Med. 2013 November; 1(4): 223-237, each of which is incorporated by reference in its entirety.

In embodiments, the chimeric proteins of the present disclosure comprise variants of the extracellular domain, which includes the ligand-binding domain, of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of DcR3, e.g., human DcR3.

In embodiments, the extracellular domain of DcR3 has the following amino acid sequence:

(SEQ ID NO: 58)
AETPTYPWRDAETGERLVCAQCPPGTFVQRPCRRDSPTTCPCPPRHYTQFWNYLERCRYCNVL
CGEREEEARACHATHNRACCRTGFFAHAGFCLEHASCPPGAGVIAPGTPSQNTQCPCPPGTFSA
SSSSSEQCQPHRNCTALGLALNVPGSSSHDTLC.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol. 2003 Feb. 15, 65(4):657-67, each of which is incorporated by reference in its entirety.

In embodiments, a chimeric protein of the present disclosure comprises: (1) a first domain comprising the amino acid sequence of SEQ ID NO: 57, (b) a second domain comprises the amino acid sequence of SEQ ID NO: 58, and (c) a linker comprises an amino acid sequence that is at least 95% identical to SEQ ID NO: 1, SEQ ID NO: 2, SEQ ID NO: 3, or the linker underlined and/or in bold in SEQ ID NO: 59 below.

In embodiments, a IL11RA-Fc-DcR3 chimeric protein of the present disclosure has the following amino acid sequence:

(SEQ ID NO: 59)
SPCPQAWGPPGVQYGQPGRSVKLCCPGVTAGDPVSWFRDGEPKLLQGPDSGLGHELVLAQAD
STDEGTYICQTLDGALGGTVTLQLGYPPARPVVSCQAADYENFSCTWSPSQISGLPTRYLTSYRK
KTVLGADSQRRSPSTGPWPCPQDPLGAARCVVHGAEFWSQYRINVTEVNPLGASTRLLDVSLQSI
LRPDPPQGLRVESVPGYPRRLRASWTYPASWPCQPHFLLKFRLQYRPAQHPAWSTVEPAGLEEV
ITDAVAGLPHAVRVSARDFLDAGTWSTWSPEAWGTPSTGTIPKEIPAWGQLHTQPEVEPQVDSPA
PPRPSLQPHPRLLDHRDSVEQVAVLASKYGPPCPPCPAPEFLGGPSVFLFPPKPKDQLMISRTPE
VTCVVVDVSQEDPEVQFNWYVDGVEVHNAKTKPREEQFNSTYRVVSVLTVLHQDWLSGKEYKC
KVSSKGLPSSIEKTISKAKGQPREPQVYTLPPSQEEMTKNQVSLTCLVKGFYPSDIAVEWESNGQP
ENNYKTTPPVLDSDGSFFLYSRLTVDKSRWQEGNVFSCSVLHEALHNHYTQKSLSLSLGKIEGRM
DAETPTYPWRDAETGERLVCAQCPPGTFVQRPCRRDSPTTCPCPPRHYTQFWNYLERCRYCNV
LCGEREEEARACHATHNRACCRTGFFAHAGFCLEHASCPPGAGVIAPGTPSQNTQCPCPPGTFS
ASSSSSEQCQPHRNCTALGLALNVPGSSSHDTLC.

In embodiments, a chimeric protein comprises a variant of a IL11RA-Fc-DcR3 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 59.

### DR3-Fc-PD-L1

In embodiments, the chimeric protein is capable of contemporaneously binding the DR3 ligand and the PD-L1 receptor. In embodiments, the DR3 ligand is TL1A and the PD-L1 receptor is PD-1. Death receptor 3 (DR3), also known as tumor necrosis factor receptor superfamily member 25 (TNFRSF25), is a cell surface receptor of the tumor necrosis factor receptor superfamily which mediates apoptotic signaling and differentiation. The DR3 receptor has been shown to stimulate NF-KR activity. PD-L1 plays a critical role in induction and maintenance of immune tolerance to self, in part, by acting as aligand for the inhibitory receptor PD-1; it modulates the activation threshold of T-cells and limits T-cell effector response, including cytotoxic T lymphocytes (CTLs) effector function. Accordingly, a chimeric protein comprising the extracellular domains of DR3 and PD-L1 is capable of contemporaneously stimulating an immune activation signal (via DR3) and activating an immune inhibitory signal (via PD-L1). In embodiments, this chimeric protein is referred to herein as DR3-Fc-PD-L1.

In embodiments, the chimeric proteins of the present disclosure comprise variants of the extracellular domain, which includes the ligand/receptor binding domain, of DR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of DR3, e.g., human DR3.

In embodiments, the extracellular domain of DR3 has the following amino acid sequence:

(SEQ ID NO: 60)
QGGTRSPRCDCAGDFHKKIGLFCCRGCPAGHYLKAPCTEPCGNSTCLVCPQDTFLAWENHHNSE
CARCQACDEQASQVALENCSAVADTRCGCKPGWFVECQVSQCVSSSPFYCQPCLDCGALHRHT
RLLCSRRDTDCGTCLPGFYEHGDGCVSCPTSTLGSCPERCAAVCGWRQ.

In embodiments, a chimeric protein comprises a variant of the extracellular domain of DR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 60.

In embodiments, the first domain of a chimeric protein comprises an amino acid sequence that is at least 95% identical to the amino acid sequence of SEQ ID NO: 60.

One of ordinary skill may select variants of the known amino acid sequence of DR3 by consulting the literature, e.g., Pereira et al., “DNA methylation polymerase chain reaction (PCR) array of apoptosis-related genes in pleomorphic adenomas of the salivary glands,” Oral Surg Oral Med Oral Pathol Oral Radiol 124 (6), 554-560 (2017); Bittner et al., “Death receptor 3 signaling enhances proliferation of human regulatory T cells,” FEBS Lett. 591 (8), 1187-1195 (2017); Screaton et al., “LARD: a new lymphoid-specific death domain containing receptor regulated by alternative pre-mRNA splicing,” Proc. Natl. Acad. Sci. U.S.A. 94 (9), 4615-4619 (1997); Hashiramoto et al., “A variant of death-receptor 3 associated with rheumatoid arthritis interferes with apoptosis-induction of T cell.” The Journal of Biological Chemistry 293: 1933-1943 (2017); Levin et al., “Directed evolution of a soluble human DR3 receptor for the inhibition of TL1A induced cytokine secretion”. PLoS One. 2017; 12(3): e0173460; and Zhicheng et al., “Aberrant expression and function of death receptor-3 and death decoy receptor-3 in human cancer.” Exp Ther Med. 2(2): 167-172 (2011), each of which is incorporated by reference in its entirety.

In embodiments, the chimeric proteins of the present disclosure comprise variants of the extracellular domain, which includes the receptor-binding domain, of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of PD-L1, e.g., human PD-L1.

In embodiments, the extracellular domain of PD-L1 has the following amino acid sequence:

(SEQ ID NO: 61)
FTVTVPKDLYWVEYGSNMTIECKFPVEKQLDLAALIVYWEMEDKNIIQFVHGEEDLKVQHSSYRQR
ARLLKDQLSLGNAALQITDVKLQDAGVYRCMISYGGADYKRITVKVNAPYNKINQRILVVDPVTSEH
ELTCQAEGYPKAEVIWTSSDHQVLSGKTTTTNSKREEKLFNVTSTLRINTTTNEIFYCTFRRLDPEE
NHTAELVIPELPLAHPPNER.

In embodiments, a chimeric protein comprises a variant of the extracellular domain of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 61.

In embodiments, the second domain of a chimeric protein comprises an amino acid sequence that is at least 95% identical to the amino acid sequence of SEQ ID NO: 61.

One of ordinary skill may select variants of the known amino acid sequence of PD-L1 by consulting the literature, e.g., Freeman et al., “Engagement of the PD-1 immunoinhibitory receptor by a novel B7-family member leads to negative regulation of lymphocyte activation.” J. Exp. Med. 192:1027-1034 (2000); Burr, et al, “CMTM6 maintains the expression of PD-L1 and regulates anti-tumour immunity.” Nature 549 (7670), 101-105 (2017); Lin et al., “The PD-1/PD-L1 complex resembles the antigen-binding Fv domains of antibodies and T cell receptors.” Proc. Natl. Acad. Sci. U.S.A. 105 (8), 3011-3016 (2008); and Zak et al., “Structure of the Complex of Human Programmed Death 1, PD-1, and Its Ligand PD-L1.” Structure 23 (12), 2341-2348 (2015), each of which is incorporated by reference in its entirety.

In embodiments, a chimeric protein of the present disclosure comprises: (1) a first domain comprising the amino acid sequence of SEQ ID NO: 60, (b) a second domain comprises the amino acid sequence of SEQ ID NO: 61, and (c) a linker comprises an amino acid sequence that is at least 95% identical to SEQ ID NO: 1, SEQ ID NO: 2, SEQ ID NO: 3, or the linker underlined and/or in bold in SEQ ID NO: 62 below.

In embodiments, a DR3-Fc-PD-L1 chimeric protein of the present disclosure has the following amino acid sequence:

(SEQ ID NO: 62)
QGGTRSPRCDCAGDFHKKIGLFCCRGCPAGHYLKAPCTEPCGNSTCLVCPQDTFLAWENHHNS
ECARCQACDEQASQVALENCSAVADTRCGCKPGWFVECQVSQCVSSSPFYCQPCLDCGALHRH
TRLLCSRRDTDCGTCLPGFYEHGDGCVSCPTSTLGSCPERCAAVCGWRQSKYGPPCPPCPAPE
FLGGPSVFLFPPKPKDQLMISRTPEVTCVVVDVSQEDPEVQFNWYVDGVEVHNAKTKPREEQFN
STYRVVSVLTVLHQDWLSGKEYKCKVSSKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNVFSCSV
LHEALHNHYTQKSLSLSLGKIEGRMDFTVTVPKDLYVVEYGSNMTIECKFPVEKQLDLAALIVYWE
MEDKNIIQFVHGEEDLKVQHSSYRQRARLLKDQLSLGNAALQITDVKLQDAGVYRCMISYGGADY
KRITVKVNAPYNKINQRILVVDPVTSEHELTCQAEGYPKAEVIWTSSDHQVLSGKTTTTNSKREEKL
FNVTSTLRINTTTNEIFYCTFRRLDPEENHTAELVIPELPLAHPPNER.

In embodiments, a chimeric protein comprises a variant of a DR3-Fc-PD-L1 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

### MADCAM-Fc-CCL20

In embodiments, the chimeric protein is capable of contemporaneously binding the MADCAM receptor and the CCL20 receptor. In embodiments, the MADCAM receptor is alpha(4)beta(7) integrin, and the CCL20 receptor is CCR6. Mucosal addressin cell adhesion molecule-1 (MADCAM) is a homing ligand preferentially expressed on gut-associated endothelial cells that plays a central role in leukocyte trafficking into the mucosal immune compartment. When bound to its receptor, CCR6, Chemokine (C-C motif) ligand 20 (CCL20) is responsible for the chemoattraction of immature dendritic cells (DC), effector/memory T-cells and B-cells. CCR6 also plays a role in promoting migration to both the skin and mucosal surfaces under homeostatic and inflammatory conditions, as well as in pathology, including cancer and rheumatoid arthritis. Accordingly, without wishing to be bound by theory, a chimeric protein comprising the extracellular domains of MADCAM and CCL20 is capable of contemporaneously competitively inhibiting activation of an integrin that facilitates attachment and migration of an immune cell across an endothelial surface (via MADCAM binding to alpha 4 beta 7 integrins, as an example) and providing an exogenous chemokine that reduces or eliminates an immune cell from sensing a chemokine gradient (via CCL20). In embodiments, this chimeric protein is referred to herein as MADCAM-Fc-CCL20.

In embodiments, the chimeric proteins of the present disclosure comprise variants of the extracellular domain, which includes the ligand/receptor binding domain, of MADCAM. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of MADCAM, e.g., human MADCAM.

In embodiments, the extracellular domain of MADCAM has the following amino acid sequence:

(SEQ ID NO: 63)
QSLQVKPLQVEPPEPWVAVALGASRQLTCRLACADRGASVQWRGLDTSLGAVQSDTGRSVLTVR
NASLSAAGTRVCVGSCGGRTFQHTVQLLVYAFPDQLTVSPAALVPGDPEVACTAHKVTPVDPNAL
SFSLLVGGQELEGAQALGPEVQEEEEEPQGDEDVLFRVTERWRLPPLGTPVPPALYCQATMRLPG
LELSHRQAIPVLHSPTSPEPPDTTSPESPDTTSPESPDTTSQEPPDTTSPEPPDKTSPEPAPQQGST
HTPRSPGSTRTRRPEISQAGPTQGEVIPTGSSKPAGDQSKYGPP.

In embodiments, a chimeric protein comprises a variant of the extracellular domain of MADCAM. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 63.

In embodiments, the first domain of a chimeric protein comprises an amino acid sequence that is at least 95% identical to the amino acid sequence of SEQ ID NO: 63.

One of ordinary skill may select variants of the known amino acid sequence of MADCAM by consulting the literature, e.g., Wyant et al., “Development and validation of receptor occupancy pharmacodynamic assays used in the clinical development of the monoclonal antibody vedolizumab,” Cytometry B Clin Cytom 90 (2), 168-176 (2016); Wang et al., “Doxorubicin induces apoptosis by targeting Madcam1 and AKT and inhibiting protein translation initiation in hepatocellular carcinoma cells,” Oncotarget 6 (27), 24075-24091 (2015); Zhang et al., “Disruption of disulfide restriction at integrin knees induces activation and ligand-independent signaling of alpha(4)beta(7),” J. Cell. Sci. 126 (Pt 21), 5030-5041 (2013); Ala et al., “Mucosal addressin cell adhesion molecule (MAdCAM-1) expression is upregulated in the cirrhotic liver and immunolocalises to the peribiliary plexus and lymphoid aggregates,” Dig. Dis. Sci. 58 (9), 2528-2541 (2013); Yu et al., “Domain 1 of mucosal addressin cell adhesion molecule has an I1-set fold and a flexible integrin-binding loop,” J. Biol. Chem. 288 (9), 6284-6294 (2013); Leung et al., “Genomic organization, chromosomal mapping, and analysis of the 5′ promoter region of the human MAdCAM-1 gene,” Immunogenetics 46 (2), 111-119 (1997); Briskin et al., “MAdCAM-1 has homology to immunoglobulin and mucin-like adhesion receptors and to IgA1,” Nature 363 (6428), 461-464 (1993), each of which is incorporated by reference in its entirety.

In embodiments, the chimeric proteins of the present disclosure comprise variants of the extracellular domain, which includes the receptor-binding domain, of CCL20. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of CCL20, e.g., human CCL20.

In embodiments, the extracellular domain of CCL20 has the following amino acid sequence:

(SEQ ID NO: 64)
ASNFDCCLGYTDRILHPKFIVGFTRQLANEGCDINAIIFHTKKKLSVC
ANPKQTWVKYIVRLLSKKVKNM.

In embodiments, a chimeric protein comprises a variant of the extracellular domain of CCL20. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at 25 least about 97%, or at least about 98%, or at least about 99% sequence identity with SEQ ID NO: 64.

In embodiments, the second domain of a chimeric protein comprises an amino acid sequence that is at least 95% identical to the amino acid sequence of SEQ ID NO: 64.

One of ordinary skill may select variants of the known amino acid sequence of CCL20 by consulting the literature, e.g., Su et al., “CCL20 Promotes Ovarian Cancer Chemotherapy Resistance by Regulating ABCB1 Expression,” Cell Struct. Funct. 44 (1), 21-28 (2019); Schutyser et al., “The CC chemokine CCL20 and its receptor CCR6,” Cytokine Growth Factor Rev. 14(5):409-26 (2003); Zhao et al., “Stromal Cell-Derived CCL20 Promotes Tumor Progression and Osteolysis in Giant Cell Tumor of Bone,” Cell. Physiol. Biochem. 51 (5), 2472-2483 (2018); Baba et al., “Identification of CCR6, the specific receptor for a novel lymphocyte-directed CC chemokine LARC,” J. Biol. Chem. 272 (23), 14893-14898 (1997), each of which is incorporated by reference in its entirety.

In embodiments, a chimeric protein of the present disclosure comprises: (1) a first domain comprising the amino acid sequence of SEQ ID NO: 63, (b) a second domain comprises the amino acid sequence of SEQ ID NO: 64, and (c) a linker comprises an amino acid sequence that is at least 95% identical to SEQ ID NO: 1, SEQ ID NO: 2, SEQ ID NO: 3, or the linker underlined and/or in bold in SEQ ID NO: 65 below.

In embodiments, a MADCAM-Fc-CCL20 chimeric protein of the present disclosure has the following amino acid sequence:

(SEQ ID NO: 65)
QSLQVKPLQVEPPEPVVAVALGASRQLTCRLACADRGASVQWRGLDTSLGAVQSDTGRSVLTVR
NASLSAAGTRVCVGSCGGRTFQHTVQLLVYAFPDQLTVSPAALVPGDPEVACTAHKVTPVDPNAL
SFSLLVGGQELEGAQALGPEVQEEEEEPQGDEDVLFRVTERWRLPPLGTPVPPALYCQATMRLP
GLELSHRQAIPVLHSPTSPEPPDTTSPESPDTTSPESPDTTSQEPPDTTSPEPPDKTSPEPAPQQG
STHTPRSPGSTRTRRPEISQAGPTQGEVIPTGSSKPAGDQSKYGPPCPPCPAPEFLGGPSVFLFP
PKPKDQLMISRTPEVTCVVVDVSQEDPEVQFNWYVDGVEVHNAKTKPREEQFNSTYRVVSVLTVL
HQDWLSGKEYKCKVSSKGLPSSIEKTISKAKGQPREPQVYTLPPSQEEMTKNQVSLTCLVKGFYP
SDIAVEWESNGQPENNYKTTPPVLDSDGSFFLYSRLTVDKSRWQEGNVFSCSVLHEALHNHYTQ
KSLSLSLGKIEGRMDASNFDCCLGYTDRILHPKFIVGFTRQLANEGCDINAIIFHTKKKLSVCANPKQ
TWVKYIVRLLSKKVKNM.

In embodiments, a chimeric protein comprises a variant of a MADCAM-Fc-CCL20 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 65.

### MADCAM-Fc-CCL25

In embodiments, the chimeric protein is capable of contemporaneously binding the MADCAM receptor and the CCL25 receptor. In embodiments, the MADCAM receptor is alpha(4)beta(7) integrin, and the CCL25 receptor is CCR9. Mucosal addressin cell adhesion molecule-1 (MADCAM) is a homing ligand preferentially expressed on gut-associated endothelial cells that plays a central role in leukocyte trafficking into the mucosal immune compartment. When bound to its receptor, CCR9, Chemokine (C-C motif) ligand 25 (CCL25) is responsible for mediating lymphocyte recruitment to the small intestine, in the development of the small intestinal T-cell receptor-gamma delta T-cell compartment and also may be involved in the selective homing of conventional T cells to the small intestine. Accordingly, without wishing to be bound by theory, a chimeric protein comprising the extracellular domains of MADCAM and CCL25 is capable of contemporaneously competitively inhibiting activation of an integrin that facilitates attachment and migration of an immune cell across an endothelial surface (via MADCAM binding to alpha 4 beta 7 integrins, as an example) and providing an exogenous chemokine that reduces or eliminates an immune cell from sensing a chemokine gradient (via CCL25). In embodiments, this chimeric protein is referred to herein as MADCAM-Fc-CCL25.

In embodiments, the chimeric proteins of the present disclosure comprise variants of the extracellular domain, which includes the receptor/ligand binding domain, of MADCAM. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of MADCAM, e.g., human MADCAM.

In embodiments, the extracellular domain of MADCAM has the amino acid sequence of SEQ ID NO: 63.

In embodiments, a chimeric protein comprises a variant of the extracellular domain of MADCAM. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 63.

In embodiments, the first domain of a chimeric protein comprises an amino acid sequence that is at least 95% identical to the amino acid sequence of SEQ ID NO: 63.

One of ordinary skill may select variants of the known amino acid sequence of MADCAM by consulting the literature, e.g., Wyant et al., “Development and validation of receptor occupancy pharmacodynamic assays used in the clinical development of the monoclonal antibody vedolizumab,” Cytometry B Clin Cytom 90 (2), 168-176 (2016); Wang et al., “Doxorubicin induces apoptosis by targeting Madcam1 and AKT and inhibiting protein translation initiation in hepatocellular carcinoma cells,” Oncotarget 6 (27), 24075-24091 (2015); Zhang et al., “Disruption of disulfide restriction at integrin knees induces activation and ligand-independent signaling of alpha(4)beta(7),” J. Cell. Sci. 126 (Pt 21), 5030-5041 (2013); Ala et al., “Mucosal addressin cell adhesion molecule (MAdCAM-1) expression is upregulated in the cirrhotic liver and immunolocalises to the peribiliary plexus and lymphoid aggregates,” Dig. Dis. Sci. 58 (9), 2528-2541 (2013); Yu et al., “Domain 1 of mucosal addressin cell adhesion molecule has an I1-set fold and a flexible integrin-binding loop,” J. Biol. Chem. 288 (9), 6284-6294 (2013); Leung et al., “Genomic organization, chromosomal mapping, and analysis of the 5′ promoter region of the human MAdCAM-1 gene,” Immunogenetics 46 (2), 111-119 (1997); Briskin et al., “MAdCAM-1 has homology to immunoglobulin and mucin-like adhesion receptors and to IgA1,” Nature 363 (6428), 461-464 (1993), each of which is incorporated by reference in its entirety.

In embodiments, the chimeric proteins of the present disclosure comprise variants of the extracellular domain, which includes the receptor-binding domain, of CCL25. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of CCL25, e.g., human CCL25.

In embodiments, the extracellular domain of CCL25 has the following amino acid sequence:

(SEQ ID NO: 66)
QGVFEDCCLAYHYPIGWAVLRRAWTYRIQEVSGSCNLPAAIFYLPKRH
RKVCGNPKSREVQRAMKLLDARNKVFAKLHHNTQTFQAGPHAVKKLSS
GNSKLSSSKFSNPISSSKRNVSLLISANSGL.

In embodiments, a chimeric protein comprises a variant of the extracellular domain of CCL25. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 66.

In embodiments, the second domain of a chimeric protein comprises an amino acid sequence that is at least 95% identical to the amino acid sequence of SEQ ID NO: 66.

One of ordinary skill may select variants of the known amino acid sequence of CCL25 by consulting the literature, e.g., Svensson et al., “Role of CCL25/CCR9 in immune homeostasis and disease,” Expert Rev Clin Immunol. 2(5):759-73. doi: 10.1586/1744666X.2.5.759 (2006); Von Hundelshausen et al., “Chemokine interactome mapping enables tailored intervention in acute and chronic inflammation,” Sci Transl Med 9 (384) (2017); Zhang et al., “CCL25/CCR9 Signal Promotes Migration and Invasion in Hepatocellular and Breast Cancer Cell Lines,” DNA Cell Biol. 35 (7), 348-357 (2016); Zaballos et al., “Cutting edge: identification of the orphan chemokine receptor GPR-9-6 as CCR9, the receptor for the chemokine TECK,” J. Immunol. 162 (10), 5671-5675 (1999); Vicari et al., “TECK: a novel CC chemokine specifically expressed by thymic dendritic cells and potentially involved in T cell development,” Immunity 7 (2), 291-301 (1997), each of which is incorporated by reference in its entirety.

In embodiments, a chimeric protein of the present disclosure comprises: (1) a first domain comprising the amino acid sequence of SEQ ID NO: 63, (b) a second domain comprises the amino acid sequence of SEQ ID NO: 66, and (c) a linker comprises an amino acid sequence that is at least 95% identical to SEQ ID NO: 1, SEQ ID NO: 2, SEQ ID NO: 3, or the linker underlined and/or in bold in SEQ ID NO: 67 below.

In embodiments, a MADCAM-Fc-CCL25 chimeric protein of the present disclosure has the following amino acid sequence:

(SEQ ID NO: 67)
QSLQVKPLQVEPPEPVVAVALGASRQLTCRLACADRGASVQWRGLDTSLGAVQSDTGRSVLTVR
NASLSAAGTRVCVGSCGGRTFQHTVQLLVYAFPDQLTVSPAALVPGDPEVACTAHKVTPVDPNAL
SFSLLVGGQELEGAQALGPEVQEEEEEPQGDEDVLFRVTERWRLPPLGTPVPPALYCQATMRLP
GLELSHRQAIPVLHSPTSPEPPDTTSPESPDTTSPESPDTTSQEPPDTTSPEPPDKTSPEPAPQQG
STHTPRSPGSTRTRRPEISQAGPTQGEVIPTGSSKPAGDQSKYGPPCPPCPAPEFLGGPSVFLFP
PKPKDQLMISRTPEVTCVVVDVSQEDPEVQFNWYVDGVEVHNAKTKPREEQFNSTYRVVSVLTVL
HQDWLSGKEYKCKVSSKGLPSSIEKTISKAKGQPREPQVYTLPPSQEEMTKNQVSLTCLVKGFYP
SDIAVEWESNGQPENNYKTTPPVLDSDGSFFLYSRLTVDKSRWQEGNVFSCSVLHEALHNHYTQ
KSLSLSLGKIEGRMDQGVFEDCCLAYHYPIGWAVLRRAWTYRIQEVSGSCNLPAAIFYLPKRHRKV
CGNPKSREVQRAMKLLDARNKVFAKLHHNTQTFQAGPHAVKKLSSGNSKLSSSKFSNPISSSKRN
VSLLISANSGL.

In embodiments, a chimeric protein comprises a variant of a MADCAM-Fc-CCL25 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 67.

### MADCAM-Fc-PD-L1

In embodiments, the chimeric protein is capable of contemporaneously binding the MADCAM receptor and the PD-L1 receptor. In embodiments, the MADCAM receptor is alpha(4)beta(7) integrin and the PD-L1 receptor is PD-1. Mucosal addressin cell adhesion molecule-1 (MADCAM) is a homing ligand preferentially expressed on gut-associated endothelial cells that plays a central role in leukocyte traffic into the mucosal immune compartment. PD-L1 plays a critical role in induction and maintenance of immune tolerance to self, in part, by acting as a ligand for the inhibitory receptor PD-1; it modulates the activation threshold of T-cells and limits T-cell effector response, including cytotoxic T lymphocytes (CTLs) effector function. Accordingly, without wishing to be bound by theory, a chimeric protein comprising the extracellular domains of MADCAM and PD-L1 is capable of contemporaneously competitively integrin that facilitates attachment and migration of an immune cell across an endothelial surface (via MADCAM) and activating an immune inhibitory signal (via PD-L1). In embodiments, this chimeric protein is referred to herein as MADCAM-Fc-PD-L1.

In embodiments, the chimeric proteins of the present disclosure comprise variants of the extracellular domain, which includes the ligand-binding domain, of MADCAM. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of MADCAM, e.g., human MADCAM.

In embodiments, the extracellular domain of MADCAM has the amino acid sequence of SEQ ID NO: 63.

In embodiments, a chimeric protein comprises a variant of the extracellular domain of MADCAM. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 63.

In embodiments, the first domain of a chimeric protein comprises an amino acid sequence that is at least 95% identical to the amino acid sequence of SEQ ID NO: 63.

One of ordinary skill may select variants of the known amino acid sequence of MADCAM by consulting the literature, e.g., Wyant et al., “Development and validation of receptor occupancy pharmacodynamic assays used in the clinical development of the monoclonal antibody vedolizumab,” Cytometry B Clin Cytom 90 (2), 168-176 (2016); Wang et al., “Doxorubicin induces apoptosis by targeting Madcam1 and AKT and inhibiting protein translation initiation in hepatocellular carcinoma cells,” Oncotarget 6 (27), 24075-24091 (2015); Zhang et al., “Disruption of disulfide restriction at integrin knees induces activation and ligand-independent signaling of alpha(4)beta(7),” J. Cell. Sci. 126 (Pt 21), 5030-5041 (2013); Ala et al., “Mucosal addressin cell adhesion molecule (MAdCAM-1) expression is upregulated in the cirrhotic liver and immunolocalises to the peribiliary plexus and lymphoid aggregates,” Dig. Dis. Sci. 58 (9), 2528-2541 (2013); Yu et al., “Domain 1 of mucosal addressin cell adhesion molecule has an 11-set fold and a flexible integrin-binding loop,” J. Biol. Chem. 288 (9), 6284-6294 (2013); Leung et al., “Genomic organization, chromosomal mapping, and analysis of the 5′ promoter region of the human MAdCAM-1 gene,” Immunogenetics 46 (2), 111-119 (1997); Briskin et al., “MAdCAM-1 has homology to immunoglobulin and mucin-like adhesion receptors and to IgA1,” Nature 363 (6428), 461-464 (1993), each of which is incorporated by reference in its entirety.

In embodiments, the chimeric proteins of the present disclosure comprise variants of the extracellular domain, which includes the receptor-binding domain, of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of PD-L1, e.g., human PD-L1.

In embodiments, the extracellular domain of PD-L1 has the amino acid sequence of SEQ ID NO: 61.

In embodiments, a chimeric protein comprises a variant of the extracellular domain of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 61.

In embodiments, the second domain of a chimeric protein comprises an amino acid sequence that is at least 95% identical to the amino acid sequence of SEQ ID NO: 61.

One of ordinary skill may select variants of the known amino acid sequence of PD-L1 by consulting the literature, e.g., Freeman et al., “Engagement of the PD-1 immunoinhibitory receptor by a novel B7-family member leads to negative regulation of lymphocyte activation.” J. Exp. Med. 192:1027-1034 (2000); Burr, et al, “CMTM6 maintains the expression of PD-L1 and regulates anti-tumour immunity.” Nature 549 (7670), 101-105 (2017); Lin et al., “The PD-1/PD-L1 complex resembles the antigen-binding Fv domains of antibodies and T cell receptors.” Proc. Natl. Acad. Sci. U.S.A. 105 (8), 3011-3016 (2008); and Zak et al., “Structure of the Complex of Human Programmed Death 1, PD-1, and Its Ligand PD-L1.” Structure 23 (12), 2341-2348 (2015), each of which is incorporated by reference in its entirety.

In embodiments, a chimeric protein of the present disclosure comprises: (1) a first domain comprising the amino acid sequence of SEQ ID NO: 63, (b) a second domain comprises the amino acid sequence of SEQ ID NO: 61, and (c) a linker comprises an amino acid sequence that is at least 95% identical to SEQ ID NO: 1, SEQ ID NO: 2, SEQ ID NO: 3, or the linker underlined and/or in bold in SEQ ID NO: 68 below.

In embodiments, a MADCAM-Fc-PD-L1 chimeric protein of the present disclosure has the following amino acid sequence:

(SEQ ID NO: 68)
QSLQVKPLQVEPPEPWVAVALGASRQLTCRLACADRGASVQWRGLDTSLGAVQSDTGRSVLTVR
NASLSAAGTRVCVGSCGGRTFQHTVQLLVYAFPDQLTVSPAALVPGDPEVACTAHKVTPVDPNAL
SFSLLVGGQELEGAQALGPEVQEEEEEPQGDEDVLFRVTERWRLPPLGTPVPPALYCQATMRLP
GLELSHRQAIPVLHSPTSPEPPDTTSPESPDTTSPESPDTTSQEPPDTTSPEPPDKTSPEPAPQQG
STHTPRSPGSTRTRRPEISQAGPTQGEVIPTGSSKPAGDQSKYGPPCPPCPAPEFLGGPSVFLFP
PKPKDQLMISRTPEVTCVVVDVSQEDPEVQFNWYVDGVEVHNAKTKPREEQFNSTYRVVSVLTVL
HQDWLSGKEYKCKVSSKGLPSSIEKTISKAKGQPREPQVYTLPPSQEEMTKNQVSLTCLVKGFYP
SDIAVEWESNGQPENNYKTTPPVLDSDGSFFLYSRLTVDKSRWQEGNVFSCSVLHEALHNHYTQ
KSLSLSLGKIEGRMDFTVTVPKDLYVVEYGSNMTIECKFPVEKQLDLAALIVYWEMEDKNIIQFVHG
EEDLKVQHSSYRQRARLLKDQLSLGNAALQITDVKLQDAGVYRCMISYGGADYKRITVKVNAPYN
KINQRILVVDPVTSEHELTCQAEGYPKAEVIWTSSDHQVLSGKTTTTNSKREEKLFNVTSTLRINTT
TNEIFYCTFRRLDPEENHTAELVIPELPLAHPPNER.

In embodiments, a chimeric protein comprises a variant of a MADCAM-Fc-PD-L1 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 68.

### VCAM-Fc-PD-L1

In embodiments, the chimeric protein is capable of contemporaneously binding the VCAM receptor and the PD-L1 receptor. In embodiments, the VCAM receptors are VLA-4 and α4β7 integrins and the PD-L1 receptor is PD-1. Vascular cell adhesion molecule 1 (VCAM) is a cell surface adhesion molecule involved in the recruitment of leukocytes to endothelial cells and signal transduction. VCAM is capable of inhibiting the entry of new potentially pathogenic cells to the local microenvironment and activating an immune inhibitory signal on pathogenic cells already present. PD-L1 plays a critical role in induction and maintenance of immune tolerance to self, in part, by acting as a ligand for the inhibitory receptor PD-1; it modulates the activation threshold of T-cells and limits T-cell effector response, including cytotoxic T lymphocytes (CTLs) effector function. Accordingly, without wishing to be bound by theory, a chimeric protein comprising the extracellular domains of VCAM and PD-L1 is capable of contemporaneously competitively inhibiting an integrin that facilitates attachment and migration of an immune cell across an endothelial surface (via VCAM) and activating an immune inhibitory signal (via PD-L1). In embodiments, this chimeric protein is referred to herein as VCAM-Fc-PD-L1.

In embodiments, the chimeric proteins of the present disclosure comprise variants of the extracellular domain, which includes the ligand/receptor-binding domain, of VCAM. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of VCAM, e.g., human VCAM.

In embodiments, the extracellular domain of VCAM has the following amino acid sequence:

(SEQ ID NO: 69)
FKIETTPESRYLAQIGDSVSLTCSTTGCESPFFSWRTQIDSPLNGKVTNEGTTSTLTMNPVSFGNEH
SYLCTATCESRKLEKGIQVEIYSFPKDPEIHLSGPLEAGKPITVKCSVADVYPFDRLEIDLLKGDHLMK
SQEFLEDADRKSLETKSLEVTFTPVIEDIGKVLVCRAKLHIDEMDSVPTVRQAVKELQVYISPKNTVIS
VNPSTKLQEGGSVTMTCSSEGLPAPEIFWSKKLDNGNLQHLSGNATLTLIAMRMEDSGIYVCEGVN
LIGKNRKEVELIVQEKPFTVEISPGPRIAAQIGDSVMLTCSVMGCESPSFSWRTQIDSPLSGKVRSE
GTNSTLTLSPVSFENEHSYLCTVTCGHKKLEKGIQVELYSFPRDPEIEMSGGLVNGSSVTVSCKVPS
VYPLDRLEIELLKGETILENIEFLEDTDMKSLENKSLEMTFIPTIEDTGKALVCQAKLHIDDMEFEPKQ
RQSTQTLYVNVAPRDTTVLVSPSSILEEGSSVNMTCLSQGFPAPKILWSRQLPNGELQPLSENATLT
LISTKMEDSGVYLCEGINQAGRSRKEVELIIQVTPKDIKLTAFPSESVKEGDTVIISCTCGNVPETWIIL
KKKAETGDTVLKSIDGAYTIRKAQLKDAGVYECESKNKVGSQLRSLTLDVQGRENNKDYFSPESKY
GPP.

In embodiments, a chimeric protein comprises a variant of the extracellular domain of VCAM. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 69.

In embodiments, the first domain of a chimeric protein comprises an amino acid sequence that is at least 95% identical to the amino acid sequence of SEQ ID NO: 69.

One of ordinary skill may select variants of the known amino acid sequence of VCAM by consulting the literature, e.g., Pepinsky et al., “Structure/function studies on vascular cell adhesion molecule-1,” J. Biol. Chem. 267 (25), 17820-17826 (1992); Yousef et al. “Aged blood impairs hippocampal neural precursor activity and activates microglia via brain endothelial cell VCAM1,” Nat. Med. 25 (6), 988-1000 (2019); Choi et al., “TNF-alpha-Induced YAP/TAZ Activity Mediates Leukocyte-Endothelial Adhesion by Regulating VCAM1 Expression in Endothelial Cells,” Int J Mol Sci 19 (11), E3428 (2018); Neish et al., “Functional analysis of the human vascular cell adhesion molecule 1 Promoter,” J. Exp. Med. 176 (6), 1583-1593 (1992), each of which is incorporated by reference in its entirety.

In embodiments, the chimeric proteins of the present disclosure comprise variants of the extracellular domain, which includes the receptor-binding domain, of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of PD-L1, e.g., human PD-L1.

In embodiments, the extracellular domain of PD-L1 has the amino acid sequence of SEQ ID NO: 61.

In embodiments, a chimeric protein comprises a variant of the extracellular domain of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 61.

In embodiments, the second domain of a chimeric protein comprises an amino acid sequence that is at least 95% identical to the amino acid sequence of SEQ ID NO: 61.

One of ordinary skill may select variants of the known amino acid sequence of PD-L1 by consulting the literature, e.g., Freeman et al., “Engagement of the PD-1 immunoinhibitory receptor by a novel B7-family member leads to negative regulation of lymphocyte activation.” J. Exp. Med. 192:1027-1034 (2000); Burr, et al, “CMTM6 maintains the expression of PD-L1 and regulates anti-tumour immunity.” Nature 549 (7670), 101-105 (2017); Lin et al., “The PD-1/PD-L1 complex resembles the antigen-binding Fv domains of antibodies and T cell receptors.” Proc. Natl. Acad. Sci. U.S.A. 105 (8), 3011-3016 (2008); and Zak et al., “Structure of the Complex of Human Programmed Death 1, PD-1, and Its Ligand PD-L1.” Structure 23 (12), 2341-2348 (2015), each of which is incorporated by reference in its entirety.

In embodiments, a chimeric protein of the present disclosure comprises: (1) a first domain comprising the amino acid sequence of SEQ ID NO: 69, (b) a second domain comprises the amino acid sequence of SEQ ID NO: 61, and (c) a linker comprises an amino acid sequence that is at least 95% identical to SEQ ID NO: 1, SEQ ID NO: 2, SEQ ID NO: 3, or the linker underlined and/or in bold in SEQ ID NO: 70 below.

In embodiments, an VCAM-Fc-PD-L1 chimeric protein of the present disclosure has the following amino acid sequence:

(SEQ ID NO: 70)
FKIETTPESRYLAQIGDSVSLTCSTTGCESPFFSWRTQIDSPLNGKVTNEGTTSTLTMNPVSFGNE
HSYLCTATCESRKLEKGIQVEIYSFPKDPEIHLSGPLEAGKPITVKCSVADVYPFDRLEIDLLKGDHL
MKSQEFLEDADRKSLETKSLEVTFTPVIEDIGKVLVCRAKLHIDEMDSVPTVRQAVKELQVYISPKN
TVISVNPSTKLQEGGSVTMTCSSEGLPAPEIFWSKKLDNGNLQHLSGNATLTLIAMRMEDSGIYVC
EGVNLIGKNRKEVELIVQEKPFTVEISPGPRIAAQIGDSVMLTCSVMGCESPSFSWRTQIDSPLSGK
VRSEGTNSTLTLSPVSFENEHSYLCTVTCGHKKLEKGIQVELYSFPRDPEIEMSGGLVNGSSVTVS
CKVPSVYPLDRLEIELLKGETILENIEFLEDTDMKSLENKSLEMTFIPTIEDTGKALVCQAKLHIDDME
FEPKQRQSTQTLYVNVAPRDTTVLVSPSSILEEGSSVNMTCLSQGFPAPKILWSRQLPNGELQPLS
ENATLTLISTKMEDSGVYLCEGINQAGRSRKEVELIIQVTPKDIKLTAFPSESVKEGDTVIISCTCGNV
PETWIILKKKAETGDTVLKSIDGAYTIRKAQLKDAGVYECESKNKVGSQLRSLTLDVQGRENNKDY
FSPESKYGPPCPPCPAPEFLGGPSVFLFPPKPKDQLMISRTPEVTCVVVDVSQEDPEVQFNWYVD
GVEVHNAKTKPREEQFNSTYRVVSVLTVLHQDWLSGKEYKCKVSSKGLPSSIEKTISKAKGQPRE
PQVYTLPPSQEEMTKNQVSLTCLVKGFYPSDIAVEWESNGQPENNYKTTPPVLDSDGSFFLYSRL
TVDKSRWQEGNVFSCSVLHEALHNHYTQKSLSLSLGKIEGRMDFTVTVPKDLYVVEYGSNMTIEC
KFPVEKQLDLAALIVYWEMEDKNIIQFVHGEEDLKVQHSSYRQRARLLKDQLSLGNAALQITDVKL
QDAGVYRCMISYGGADYKRITVKVNAPYNKINQRILVVDPVTSEHELTCQAEGYPKAEVIWTSSDH
QVLSGKTTTTNSKREEKLFNVTSTLRINTTTNEIFYCTFRRLDPEENHTAELVIPELPLAHPPNER.

In embodiments, a chimeric protein comprises a variant of an VCAM-Fc-PD-L1 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 70.

### IL36R-Fc-DcR3

In embodiments, the chimeric protein is capable of contemporaneously binding the IL36R ligand and the DcR3 ligand. In embodiments, the IL36R ligand is interleukin (IL)-36, and the DcR3 ligand is Fas ligand (FasL), LIGHT, or TL1A. Interleukin 36 receptor (IL36R), also known as interleukin-1 receptor-like 2 (IL1RL2), is a member of the IL1 cytokine receptor family. Binding of IL36R with its ligand induces pro-inflammatory effects on various target cells, such as keratinocytes, synoviocytes, dendritic cells and T cells. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via‘non-decoy’ action. Upregulation of DcR3 during inflammatory reactions exerts negative-feedback to suppress inflammation. Accordingly, a chimeric protein comprising the extracellular domain of IL36R and the extracellular domain of DcR3 is capable of contemporaneously competitively inhibiting an immune activating signal (via IL36R and DcR3). In embodiments, this chimeric protein is referred to herein as IL36R-Fc-DcR3.

In embodiments, the chimeric proteins of the present disclosure comprise variants of the extracellular domain, which includes the ligand/receptor-binding domain, of IL36R. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of IL36R, e.g., human IL36R.

In embodiments, the extracellular domain of IL36R has the following amino acid sequence:

(SEQ ID NO: 71)
DGCKDIFMKNEILSASQPFAFNCTFPPITSGEVSVTWYKNSSKIPVSKIIQSRIHQDETWILFLPMEW
GDSGVYQCVIKGRDSCHRIHVNLTVFEKHWCDTSIGGLPNLSDEYKQILHLGKDDSLTCHLHFPKS
CVLGPIKWYKDCNEIKGERFTVLETRLLVSNVSAEDRGNYACQAILTHSGKQYEVLNGITVSITERAG
YGGSVPKIIYPKNHSIEVQLGTTLIVDCNVTDTKDNTNLRCWRVNNTLVDDYYDESKRIREGVETHV
SFREHNLYTVNITFLEVKMEDYGLPFMCHAGVSTAYIILQLPAPDFRSKYGPP.

In embodiments, a chimeric protein comprises a variant of the extracellular domain of IL36R. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 71.

In embodiments, the first domain of a chimeric protein comprises an amino acid sequence that is at least 95% identical to the amino acid sequence of SEQ ID NO: 71.

One of ordinary skill may select variants of the known amino acid sequence of IL36R by consulting the literature, e.g., Tomuschat et al., “Altered expression of IL36gamma and IL36 receptor (IL1RL2) in the colon of patients with Hirschsprung's disease,” Pediatr. Surg. Int. 33 (2), 181-186 (2017); Penha et al., “IL-36 receptor is expressed by human blood and intestinal T lymphocytes and is dose-dependently activated via IL-36beta and induces CD4+lymphocyte proliferation,” Cytokine 85, 18-25 (2016); Yi et al., “Structural and Functional Attributes of the Interleukin-36 Receptor,” J. Biol. Chem. 291 (32), 16597-16609 (2016), each of which is incorporated by reference in its entirety.

In embodiments, the chimeric proteins of the present disclosure comprise variants of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol. 2003 Feb. 15, 65(4):657-67, each of which is incorporated by reference in its entirety.

In embodiments, a chimeric protein of the present disclosure comprises: (1) a first domain comprising the amino acid sequence of SEQ ID NO: 71, (b) a second domain comprises the amino acid sequence of SEQ ID NO: 58, and (c) a linker comprises an amino acid sequence that is at least 95% identical to SEQ ID NO: 1, SEQ ID NO: 2, SEQ ID NO: 3, or the linker underlined and/or in bold in SEQ ID NO: 72 below.

In embodiments, a IL36R-Fc-DcR3 chimeric protein of the present disclosure has the following amino acid sequence:

(SEQ ID NO: 72)
DGCKDIFMKNEILSASQPFAFNCTFPPITSGEVSVTWYKNSSKIPVSKIIQSRIHQDETWILFLPMEW
GDSGVYQCVIKGRDSCHRIHVNLTVFEKHWCDTSIGGLPNLSDEYKQILHLGKDDSLTCHLHFPKS
CVLGPIKWYKDCNEIKGERFTVLETRLLVSNVSAEDRGNYACQAILTHSGKQYEVLNGITVSITERA
GYGGSVPKIIYPKNHSIEVQLGTTLIVDCNVTDTKDNTNLRCWRVNNTLVDDYYDESKRIREGVET
HVSFREHNLYTVNITFLEVKMEDYGLPFMCHAGVSTAYIILQLPAPDFRSKYGPPCPPCPAPEFLG
GPSVFLFPPKPKDQLMISRTPEVTCVVVDVSQEDPEVQFNWYVDGVEVHNAKTKPREEQFNSTY
RVVSVLTVLHQDWLSGKEYKCKVSSKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSVLHE
ALHNHYTQKSLSLSLGKIEGRMDAETPTYPWRDAETGERLVCAQCPPGTFVQRPCRRDSPTTCP
CPPRHYTQFWNYLERCRYCNVLCGEREEEARACHATHNRACCRTGFFAHAGFCLEHASCPPGA
GVIAPGTPSQNTQCPCPPGTFSASSSSSEQCQPHRNCTALGLALNVPGSSSHDTLC.

In embodiments, a chimeric protein comprises a variant of a IL36R-Fc-DcR3 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 72.

### IL18BP-Fc-DcR3

In embodiments, the chimeric protein is capable of contemporaneously binding the IL18BP ligand and the DcR3 ligand. In embodiments, IL18BP binds to interleukin(IL)-18, and the DcR3 ligand is Fas ligand (FasL), LIGHT, or TL1A. Interleukin-18-binding protein (IL18BP) binds to and inhibits the function of proinflammatory cytokine IL18 by preventing IL18 from binding to its receptor, thereby inhibiting IL18-induced IFN-gamma production. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Upregulation of DcR3 during inflammatory reactions exerts negative-feedback to suppress inflammation. Accordingly, a chimeric protein comprising the extracellular domain of IL18BP and the extracellular domain of DcR3 is capable of contemporaneously competitively inhibiting an immune activating signal (via IL18BP and DcR3). In embodiments, this chimeric protein is referred to herein as IL18BP-Fc-DcR3.

In embodiments, the chimeric proteins of the present disclosure comprise variants of the extracellular domain, which includes the receptor-binding domain, of IL18BP. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of IL18BP, e.g., human IL18BP.

In embodiments, the extracellular domain of IL18BP has the following amino acid sequence:

(SEQ ID NO: 73)
TPVSQTTTAATASVRSTKDPCPSQPPVFPAAKQCPALEVTWPEVEVPLNGTLSLSCVACSRFPNF
SILYWLGNGSFIEHLPGRLWEGSTSRERGSTGTQLCKALVLEQLTPALHSTNFSCVLVDPEQVVQ
RHVVLAQLWVRSPRRGLQEQEELCFHMWGGKGGLCQSSLSKYGPP.

In embodiments, a chimeric protein comprises a variant of the extracellular domain of IL18BP. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 73.

In embodiments, the second domain of a chimeric protein comprises an amino acid sequence that is at least 95% identical to the amino acid sequence of SEQ ID NO: 73.

One of ordinary skill may select variants of the known amino acid sequence of IL18BP by consulting the literature, e.g., Kim et al., “Structural requirements of six naturally occurring isoforms of the IL-18 binding protein to inhibit IL-18,” Proc. Natl. Acad. Sci. U.S.A. 97 (3), 1190-1195 (2000); Wang et al., “Altered expression of IL-18 binding protein and IL-18 receptor in basophils and mast cells of asthma patients,” Scand. J. Immunol. 87 (5), e12658 (2018); Corbaz, et al, “IL-18-binding protein expression by endothelial cells and macrophages is up-regulated during active Crohn's disease,” J. Immunol. 168 (7), 3608-3616 (2002); Paulukat et al., “Expression and release of IL-18 binding protein in response to IFN-gamma,” J. Immunol. 167 (12), 7038-7043 (2001), each of which is incorporated by reference in its entirety.

In embodiments, the chimeric proteins of the present disclosure comprise variants of the extracellular domain, which includes the ligand/receptor-binding domain, of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the first domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol, 65(4):657-67 (2003), each of which is incorporated by reference in its entirety.

In embodiments, a chimeric protein of the present disclosure comprises: (1) a first domain comprising the amino acid sequence of SEQ ID NO: 73, (b) a second domain comprises the amino acid sequence of SEQ ID NO: 58, and (c) a linker comprises an amino acid sequence that is at least 95% identical to SEQ ID NO: 1, SEQ ID NO: 2, SEQ ID NO: 3 or the linker underlined and/or in bold in SEQ ID NO: 74 below.

In embodiments, a IL18BP-Fc-DcR3 chimeric protein of the present disclosure has the following amino acid sequence:

(SEQ ID NO: 74)
TPVSQTTTAATASVRSTKDPCPSQPPVFPAAKQCPALEVTWPEVEVPLNGTLSLSCVACSRFPNF
SILYWLGNGSFIEHLPGRLWEGSTSRERGSTGTQLCKALVLEQLTPALHSTNFSCVLVDPEQVVQ
RHVVLAQLWVRSPRRGLQEQEELCFHMWGGKGGLCQSSLSKYGPPCPPCPAPEFLGGPSVFLF
PPKPKDQLMISRTPEVTCVVVDVSQEDPEVQFNWYVDGVEVHNAKTKPREEQFNSTYRVVSVLT
VLHQDWLSGKEYKCKVSSKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVLHEALHNHY
TQKSLSLSLGKIEGRMDAETPTYPWRDAETGERLVCAQCPPGTFVQRPCRRDSPTTCPCPPRHY
TQFWNYLERCRYCNVLCGEREEEARACHATHNRACCRTGFFAHAGFCLEHASCPPGAGVIAPGT
PSQNTQCPCPPGTFSASSSSSEQCQPHRNCTALGLALNVPGSSSHDTLC.

In embodiments, a chimeric protein comprises a variant of a I18BP-Fc-DcR3 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 74.

### DcR3-Fc-IL18BP

In embodiments, the chimeric protein is capable of contemporaneously binding the DcR3 ligand and the IL18BP ligand. In embodiments, the DcR3 ligand is Fas ligand (FasL), LIGHT, or TL1A and IL18BP binds to interleukin(IL)-18. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Upregulation of DcR3 during inflammatory reactions exerts negative-feedback to suppress inflammation. Interleukin-18-binding protein (IL18BP) binds to and inhibits the function of proinflammatory cytokine IL18 by preventing IL18 from binding to its receptor, thereby inhibiting IL18-induced IFN-gamma production. Accordingly, a chimeric protein comprising the extracellular domain of DcR3 and the extracellular domain of IL18BP is capable of contemporaneously competitively inhibiting an immune activating signal (via DcR3 and IL18BP). In embodiments, this chimeric protein is referred to herein as DcR3-Fc-IL18BP.

In embodiments, the chimeric proteins of the present disclosure comprise variants of the extracellular domain, which includes the ligand/receptor binding domain, of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the first domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol, 65(4):657-67 (2003), each of which is incorporated by reference in its entirety.

In embodiments, the chimeric proteins of the present disclosure comprises variants of the extracellular domain of IL18BP. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the portion IL18BP, e.g., human IL18BP.

In embodiments, the portion of IL18BP comprising its receptor-binding domain, relevant to the present disclosure, has an amino acid sequence of SEQ ID NO: 73.

In embodiments, a chimeric protein comprises a variant of the extracellular domain of IL18BP. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 73.

In embodiments, the second domain of a chimeric protein comprises an amino acid sequence that is at least 95% identical to the amino acid sequence of SEQ ID NO: 73.

One of ordinary skill may select variants of the known amino acid sequence of IL18BP by consulting the literature, e.g., Kim et al., “Structural requirements of six naturally occurring isoforms of the IL-18 binding protein to inhibit IL-18,” Proc. Natl. Acad. Sci. U.S.A. 97 (3), 1190-1195 (2000); Wang et al., “Altered expression of IL-18 binding protein and IL-18 receptor in basophils and mast cells of asthma patients,” Scand. J. Immunol. 87 (5), e12658 (2018); Corbaz, et al, “IL-18-binding protein expression by endothelial cells and macrophages is up-regulated during active Crohn's disease,” J. Immunol. 168 (7), 3608-3616 (2002); Paulukat et al., “Expression and release of IL-18 binding protein in response to IFN-gamma,” J. Immunol. 167 (12), 7038-7043 (2001), each of which is incorporated by reference in its entirety.

In embodiments, a chimeric protein of the present disclosure comprises: (1) a first domain comprising the amino acid sequence of SEQ ID NO: 58, (b) a second domain comprises the amino acid sequence of SEQ ID NO: 73, and (c) a linker comprises an amino acid sequence that is at least 95% identical to SEQ ID NO: 1, SEQ ID NO: 2, SEQ ID NO: 3, or the linker underlined and/or in bold in SEQ ID NO: 75 below.

In embodiments, a DcR3-Fc-IL18BP chimeric protein of the present disclosure has the following amino acid sequence:

(SEQ ID NO: 75)
AETPTYPWRDAETGERLVCAQCPPGTFVQRPCRRDSPTTCPCPPRHYTQFWNYLERCRYCNVL
CGEREEEARACHATHNRACCRTGFFAHAGFCLEHASCPPGAGVIAPGTPSQNTQCPCPPGTFSA
SSSSSEQCQPHRNCTALGLALNVPGSSSHDTLCSKYGPPCPPCPAPEFLGGPSVFLFPPKPKDQL
MISRTPEVTCVVVDVSQEDPEVQFNWYVDGVEVHNAKTKPREEQFNSTYRVVSVLTVLHQDWLS
GKEYKCKVSSKGLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVLHEALHNHYTQKSLSLSLG
KIEGRMDTPVSQTTTAATASVRSTKDPCPSQPPVFPAAKQCPALEVTWPEVEVPLNGTLSLSCVA
CSRFPNFSILYWLGNGSFIEHLPGRLWEGSTSRERGSTGTQLCKALVLEQLTPALHSTNFSCVLVD
PEQVVQRHVVLAQLWVRSPRRGLQEQEELCFHMWGGKGGLCQSSL.

In embodiments, a chimeric protein comprises a variant of a DcR3-Fc-IL18BP chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 75.

### OSMR-Alpha-DcR3

In embodiments, the chimeric protein is capable of contemporaneously binding the OSMR ligand and the DcR3 ligand. In embodiments, the OSMR ligand is Oncostatin M (OSM) and the DcR3 ligand is the DcR3 ligand is Fas ligand (FasL), LIGHT, or TL1A. Oncostatin M receptor (OSMR) is a member of the type I cytokine receptor family. OSMR heterodimerizes with interleukin 6 signal transducer to form the type II oncostatin M receptor and with interleukin 31 receptor A to form the interleukin 31 receptor, and thus transduces oncostatin M and interleukin 31-induced signaling events. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Upregulation of DcR3 during inflammatory reactions exerts negative-feedback to suppress inflammation. Accordingly, a chimeric protein comprising the extracellular domains of OSMR and of DcR3 is capable of contemporaneously competitively inhibiting an immune activating signal (via OSMR and DcR3). In embodiments, this chimeric protein is referred to herein as OSMR-Alpha-DcR3.

In embodiments, the chimeric proteins of the present disclosure comprise variants of the extracellular domain, which includes the ligand/receptor binding domain, of OSMR. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of OSMR, e.g., human OSMR.

In embodiments, the extracellular domain of OSMR has the following amino acid sequence:

(SEQ ID NO: 76)
ERLPLTPVSLKVSTNSTRQSLHLQWTVHNLPYHQELKMVFQIQISRIETSNVIWVGNYSTTVKWNQV
LHWSWESELPLECATHFVRIKSLVDDAKFPEPNFWSNWSSWEEVSVQDSTGQDILFVFPKDKLVE
EGTNVTICYVSRNIQNNVSCYLEGKQIHGEQLDPHVTAFNLNSVPFIRNKGTNIYCEASQGNVSEGM
KGIVLFVSKVLEEPKDFSCETEDFKTLHCTWDPGTDTALGWSKQPSQSYTLFESFSGEKKLCTHKN
WCNWQITQDSQETYNFTLIAENYLRKRSVNILFNLTHRVYLMNPFSVNFENVNATNAIMTWKVHSIR
NNFTYLCQIELHGEGKMMQYNVSIKVNGEYFLSELEPATEYMARVRCADASHFWKWSEWSGQNF
TTLEAAPSEAPDVWRIVSLEPGNHTVTLFWKPLSKLHANGKILFYNVVVENLDKPSSSELHSIPAPAN
STKLILDRCSYQICVIANNSVGASPASVIVISADPENKEVEEERIAGTEGGFSLSWKPQPGDVIGYVV
DWCDHTQDVLGDFQWKNVGPNTTSTVISTDAFRPGVRYDFRIYGLSTKRIACLLEKKTGYSQELAP
SDNPHVLVDTLTSHSFTLSWKDYSTESQPGFIQGYHVYLKSKARQCHPRFEKAVLSDGSECCKYKI
DNPEEKALIVDNLKPESFYEFFITPFTSAGEGPSATFTKVTTPDEHSSM.

In embodiments, a chimeric protein comprises a variant of the extracellular domain of OSMR. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 76.

In embodiments, the first domain of a chimeric protein comprises an amino acid sequence that is at least 95% identical to the amino acid sequence of SEQ ID NO: 76.

One of ordinary skill may select variants of the known amino acid sequence of OSMR by consulting the literature, e.g., Deng et al., “The role of oncostatin M receptor gene polymorphisms in bladder cancer,” World J Surg Oncol 17 (1), 30 (2019); Adrian-Segarra et al., “The AB loop and D-helix in binding site III of human Oncostatin M (OSM) are required for OSM receptor activation,” J. Biol. Chem. 293 (18), 7017-7029 (2018); Liu et al., “Oncostatin M-specific receptor expression and function in regulating cell proliferation of normal 10 and malignant mammary epithelial cells,” Cytokine 10 (4), 295-302 (1998); Auguste et al., “Signaling of type II oncostatin M receptor,” J. Biol. Chem. 272 (25), 15760-15764 (1997); Mosley et al., “Dual oncostatin M (OSM) receptors. Cloning and characterization of an alternative signaling subunit conferring OSM-specific receptor activation,” J. Biol. Chem. 271 (51), 32635-32643 (1996), each of which is incorporated by reference in its entirety.

In embodiments, the chimeric proteins of the present disclosure comprise the extracellular domain of DcR3 which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol, 65(4):657-67 (2003), each of which is incorporated by reference in its entirety.

In embodiments, a chimeric protein of the present disclosure comprises: (1) a first domain comprising the amino acid sequence of SEQ ID NO: 76, (b) a second domain comprises the amino acid sequence of SEQ ID NO: 58, and (c) a linker comprises an amino acid sequence that is at least 95% identical to SEQ ID NO: 1, SEQ ID NO: 2, SEQ ID NO: 3, or the linker underlined and/or in bold in SEQ ID NO: 77 below.

In embodiments, an OSMR-Alpha-DcR3 chimeric protein of the present disclosure has the following amino acid sequence:

(SEQ ID NO: 77)
ERLPLTPVSLKVSTNSTRQSLHLQWTVHNLPYHQELKMVFQIQISRIETSNVIWVGNYSTTVKWNQ
VLHWSWESELPLECATHFVRIKSLVDDAKFPEPNFWSNWSSWEEVSVQDSTGQDILFVFPKDKLV
EEGTNVTICYVSRNIQNNVSCYLEGKQIHGEQLDPHVTAFNLNSVPFIRNKGTNIYCEASQGNVSE
GMKGIVLFVSKVLEEPKDFSCETEDFKTLHCTWDPGTDTALGWSKQPSQSYTLFESFSGEKKLCT
HKNWCNWQITQDSQETYNFTLIAENYLRKRSVNILFNLTHRVYLMNPFSVNFENVNATNAIMTWKV
HSIRNNFTYLCQIELHGEGKMMQYNVSIKVNGEYFLSELEPATEYMARVRCADASHFWKWSEWS
GQNFTTLEAAPSEAPDVWRIVSLEPGNHTVTLFWKPLSKLHANGKILFYNVVVENLDKPSSSELHSI
PAPANSTKLILDRCSYQICVIANNSVGASPASVIVISADPENKEVEEERIAGTEGGFSLSWKPQPGD
VIGYVVDWCDHTQDVLGDFQWKNVGPNTTSTVISTDAFRPGVRYDFRIYGLSTKRIACLLEKKTGY
SQELAPSDNPHVLVDTLTSHSFTLSWKDYSTESQPGFIQGYHVYLKSKARQCHPRFEKAVLSDGS
ECCKYKIDNPEEKALIVDNLKPESFYEFFITPFTSAGEGPSATFTKVTTPDEHSSMGSGSRKGGKR
GSKYGPPCPPCPAPEFLGGPSVFLFPPKPKDQLMISRTPEVTCVVVDVSQEDPEVQFNWYVDGV
EVHNAKTKPREEQFNSTYRVVSVLTVLHQDWLSGKEYKCKVSSKGLPSSIEKTISKAKGQPREPQ
VYTLPPSQEEMTKNQVSLTCLVKGFYPSDIAVEWESNGQPENNYKTTPPVLDSDGSFFLYSRLTV
DKSRWQEGNVFSCSVLHEALHNHYTQKSLSLSLGKDEGGEDGSGSRAETPTYPWRDAETGERL
VCAQCPPGTFVQRPCRRDSPTTCPCPPRHYTQFWNYLERCRYCNVLCGEREEEARACHATHNR
ACCRTGFFAHAGFCLEHASCPPGAGVIAPGTPSQNTQCPCPPGTFSASSSSSEQCQPHRNCTAL
GLALNVPGSSSHDTLC.

In embodiments, a chimeric protein comprises a variant of a OSMR-Alpha-DcR3 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 77.

### GP130-Beta-DcR3

In embodiments, the chimeric protein is capable of contemporaneously binding the gp130 ligand and the DcR3 ligand. In embodiments, the gp130 ligands include the IL-6 family of cytokines, and the DcR3 ligand is 30 the DcR3 ligand is Fas ligand (FasL), LIGHT, or TL1A. Glycoprotein 130 (gp130), with homology to interleukin-31 receptor subunit alpha, associates with the IL-6 family of cytokines as a common signal transducer within their receptor complex that is required for signaling to regulate a variety of complex biological processes, including hematopoiesis, immune response, inflammation, proliferation, differentiation, mammalian reproduction, cardiovascular action, and neuronal survival. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Without wishing to be bound by theory, upregulation of DcR3 during inflammatory reactions exerts negative-feedback to suppress inflammation. Accordingly, a chimeric protein comprising the extracellular domains of gp130 and of DcR3 is capable of contemporaneously competitively inhibiting an immune activating signal (via gp130 and DcR3). In embodiments, this chimeric protein is referred to herein as gp130-Beta-DcR3.

In embodiments, the chimeric proteins of the present disclosure comprise variants of the extracellular domain, which includes the ligand-binding domain, of gp130. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of gp130, e.g., human gp130.

In embodiments, the extracellular domain of gp130 has the following amino acid sequence:

(SEQ ID NO: 78)
ALPAKPENISCVYYYRKNLTCTWSPGKETSYTQYTVKRTYAFGEKHDNCTTNSSTSENRASCSFFL
PRITIPDNYTIEVEAENGDGVIKSHMTYWRLENIAKTEPPKIFRVKPVLGIKRMIQIEWIKPELAPVSSD
LKYTLRFRTVNSTSWMEVNFAKNRKDKNQTYNLTGLQPFTEYVIALRCAVKESKFWSDWSQEKMG
MTEEEAPCGLELWRVLKPAEADGRRPVRLLWKKARGAPVLEKTLGYNIWYYPESNTNLTETMNTT
NQQLELHLGGESFWVSMISYNSLGKSPVATLRIPAIQEKSFQCIEVMQACVAEDQLVVKWQSSALD
VNTWMIEWFPDVDSEPTTLSWESVSQATNWTIQQDKLKPFWCYNISVYPMLHDKVGEPYSIQAYA
KEGVPSEGPETKVENIGVKTVTITWKEIPKSERKGIICNYTIFYQAEGGKGFSKTVNSSILQYGLESLK
RKTSYIVQVMASTSAGGTNGTSINFKTLSFSVFE.

In embodiments, a chimeric protein comprises a variant of the extracellular domain of gp130. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 78.

In embodiments, the first domain of a chimeric protein comprises an amino acid sequence that is at least 95% identical to the amino acid sequence of SEQ ID NO: 78.

One of ordinary skill may select variants of the known amino acid sequence of gp130 by consulting the literature, e.g., Dreuw et al., “Characterization of the signaling capacities of the novel gp130-like cytokine receptor,” J. Biol. Chem. 279 (34), 36112-36120 (2004); Diveu et al., “GPL, a novel cytokine receptor related to GP130 and leukemia inhibitory factor receptor,” J. Biol. Chem. 278 (50), 49850-49859 (2003), each of which is incorporated by reference in its entirety.

In embodiments, the chimeric proteins of the present disclosure comprise the extracellular domain of DcR3 which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol, 65(4):657-67 (2003), each of which is incorporated by reference in its entirety.

In embodiments, a chimeric protein of the present disclosure comprises: (1) a first domain comprising the amino acid sequence of SEQ ID NO: 78, (b) a second domain comprises the amino acid sequence of SEQ ID NO: 58, and (c) a linker comprises an amino acid sequence that is at least 95% identical to SEQ ID NO: 1, SEQ ID NO: 2, SEQ ID NO: 3, or the linker underlined and/or in bold in SEQ ID NO: 79 below.

In embodiments, a gp130-Beta-DcR3 chimeric protein of the present disclosure has the following amino acid sequence:

(SEQ ID NO: 79)
ALPAKPENISCVYYYRKNLTCTWSPGKETSYTQYTVKRTYAFGEKHDNCTTNSSTSENRASCSFF
LPRITIPDNYTIEVEAENGDGVIKSHMTYWRLENIAKTEPPKIFRVKPVLGIKRMIQIEWIKPELAPVS
SDLKYTLRFRTVNSTSWMEVNFAKNRKDKNQTYNLTGLQPFTEYVIALRCAVKESKFWSDWSQE
KMGMTEEEAPCGLELWRVLKPAEADGRRPVRLLWKKARGAPVLEKTLGYNIWYYPESNTNLTET
MNTTNQQLELHLGGESFWVSMISYNSLGKSPVATLRIPAIQEKSFQCIEVMQACVAEDQLVVKWQ
SSALDVNTWMIEWFPDVDSEPTTLSWESVSQATNWTIQQDKLKPFWCYNISVYPMLHDKVGEPY
SIQAYAKEGVPSEGPETKVENIGVKTVTITWKEIPKSERKGIICNYTIFYQAEGGKGFSKTVNSSILQ
YGLESLKRKTSYIVQVMASTSAGGTNGTSINFKTLSFSVFEGSGSDEGGEDGSKYGPPCPPCPAP
EFLGGPSVFLFPPKPKDQLMISRTPEVTCVVVDVSQEDPEVQFNWYVDGVEVHNAKTKPREEQF
NSTYRVVSVLTVLHQDWLSGKEYKCKVSSKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGNVFSC
SVLHEALHNHYTQKSLSLSLGKRKGGKRGSGSRAETPTYPWRDAETGERLVCAQCPPGTFVQRP
CRRDSPTTCPCPPRHYTQFWNYLERCRYCNVLCGEREEEARACHATHNRACCRTGFFAHAGFC
LEHASCPPGAGVIAPGTPSQNTQCPCPPGTFSASSSSSEQCQPHRNCTALGLALNVPGSSSHDTL
C

In embodiments, a chimeric protein comprises a variant of a gp130-Beta-DcR3 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 79.

### DcR3-Alpha-IL12A

In embodiments, the chimeric protein is capable of contemporaneously binding the DcR3 ligand and the IL12A ligand/receptor. In embodiments, the DcR3 ligand is Fas ligand (FasL), LIGHT, or TL1A and IL12A associates with IL12B. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Upregulation of DcR3 during inflammatory reactions exerts negative-feedback to suppress inflammation. Interleukin-12 subunit alpha (IL12A) associates with IL27B to form interleukin (IL)-35, which is a cytokine that is produced by regulatory lymphocytes and plays a central role in the generation of non-cannoical regulatory T cells. Accordingly, without wishing to be bound by theory, a chimeric protein comprising the extracellular domain of DcR3 and the extracellular domain of IL35 is capable of contemporaneously competitively inhibiting an immune activating signal (via DcR3) and promoting an immune regulatory signal (via IL35). In embodiments, this chimeric protein is referred to herein as DcR3-Alpha-IL12A.

In embodiments, the chimeric proteins of the present disclosure comprise variants of the extracellular domain, which includes the ligand-binding domain, of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the first domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol, 65(4):657-67 (2003), each of which is incorporated by reference in its entirety.

In embodiments, the chimeric proteins of the present disclosure comprise the extracellular domain of IL12A which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of IL12A, e.g., human IL12A.

In embodiments, the extracellular domain of 1L12A has the following amino acid sequence:

(SEQ ID NO: 80)
RNLPVATPDPGMFPCLHHSQNLLRAVSNMLQKARQTLEFYPCTSEEIDH
EDITKDKTSTVEACLPLELTKNESCLNSRETSFITNGSCLASRKTSFMM
ALCLSSIYEDLKMYQVEFKTMNAKLLMDPKRQIFLDQNMLAVIDELMQA
LNFNSETVPQKSSLEEPDFYKTKIKLCILLHAFRIRAVTIDRVMSYLNA
S.

In embodiments, a chimeric protein comprises a variant of the extracellular domain of IL12A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 80.

In embodiments, the second domain of a chimeric protein comprises an amino acid sequence that is at least 95% identical to the amino acid sequence of SEQ ID NO: 80.

One of ordinary skill may select variants of the known amino acid sequence of IL12A by consulting the literature, e.g., Wu et al., “The Contribution of Interleukin-12 Genetic Variations to Taiwanese Lung Cancer Anticancer Res. 38 (11), 6321-6327 (2018); D'Andrea et al., “Production of natural killer cell stimulatory factor (interleukin 12) by peripheral blood mononuclear cells,” J. Exp. Med. 176 (5), 1387-1398 (1992); Sieburth et al., “Assignment of genes encoding a unique cytokine (IL12) composed of two unrelated subunits to chromosomes 3 and 5,” Genomics 14 (1), 59-62 (1992); Schoenhaut et al, “Cloning and expression of murine IL-12,” J. Immunol. 148 (11), 3433-3440 (1992), each of which is incorporated by reference in its entirety.

In embodiments, a chimeric protein of the present disclosure comprises: (1) a first domain comprising the amino acid sequence of SEQ ID NO: 58, (b) a second domain comprises the amino acid sequence of SEQ ID NO: 80, and (c) a linker comprises an amino acid sequence that is at least 95% identical to SEQ ID NO: 1, SEQ ID NO: 2, SEQ ID NO: 3, or the linker underlined and/or in bold in SEQ ID NO: 81 below.

In embodiments, a DcR3-Alpha-IL12Chimeric protein of the present disclosure has the following amino acid sequence:

(SEQ ID NO: 81)
AETPTYPWRDAETGERLVCAQCPPGTFVQRPCRRDSPTTCPCPPRHYTQFWNYLERCRYCNVL
CGEREEEARACHATHNRACCRTGFFAHAGFCLEHASCPPGAGVIAPGTPSQNTQCPCPPGTFSA
SSSSSEQCQPHRNCTALGLALNVPGSSSHDTLCGSGSRKGGKRGSKYGPPCPPCPAPEFLGGP
SVFLFPPKPKDQLMISRTPEVTCVVVDVSQEDPEVQFNWYVDGVEVHNAKTKPREEQFNSTYRV
VSVLTVLHQDWLSGKEYKCKVSSKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQEGNVFSCSVLHEAL
HNHYTQKSLSLSLGKDECGEDGSGSRNLPVATPDPGMFPCLHHSQNLLRAVSNMLQKARQTLEF
YPCTSEEIDHEDITKDKTSTVEACLPLELTKNESCLNSRETSFITNGSCLASRKTSFMMALCLSSIYE
DLKMYQVEFKTMNAKLLMDPKRQIFLDQNMLAVIDELMQALNFNSETVPQKSSLEEPDFYKTKIKL
CILLHAFRIRAVTIDRVMSYLNAS

In embodiments, a chimeric protein comprises a variant of a DcR3-Alpha-IL12A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 81.

### DcR3-Beta-IL27B

In embodiments, the chimeric protein is capable of contemporaneously binding the DcR3 ligand and the IL27B ligand/receptor. In embodiments, the DcR3 ligand is Fas ligand (FasL), LIGHT, or TL1A and IL27B associates with IL12A to form IL-35. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Upregulation of DcR3 during inflammatory reactions exerts negative-feedback to suppress inflammation. Interleukin-27 subunit beta (IL27B), also known as Epstein-Barr virus induced gene 3 (EBI3), associates with IL12A to form the IL-35 interleukin, a heterodimeric cytokine which functions to promote a non-cannoical regulatory phenotype in T lymphocytes. IL-35 exhibits anti-inflammatory properties, that can regulate T-helper cell development and suppress T-cell proliferation, Accordingly, a chimeric protein comprising the extracellular domain of DcR3 and the extracellular domain of IL27B is capable of contemporaneously competitively inhibiting an immune activating signal (via DcR3) and activating an immune inhibitory signal (via IL27B). In embodiments, this chimeric protein is referred to herein as DcR3-Beta-IL27B.

In embodiments, the chimeric proteins of the present disclosure comprise variants of the extracellular domain, which includes the ligand-binding domain, of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the first domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol, 65(4):657-67 (2003), each of which is incorporated by reference in its entirety.

In embodiments, the chimeric proteins of the present disclosure comprise the extracellular domain of IL27B which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of IL27B, e.g., human IL27B.

In embodiments, the extracellular domain of IL27B has the following amino acid sequence:

(SEQ ID NO: 82)
RKGPPAALTLPRVQCRASRYPIAVDCSWTLPPAPNSTSPVSFIATYRLGMAARGHSWPCLQQTPTS
TSCTITDVQLFSMAPYVLNVTAVHPWGSSSSFVPFITEHIIKPDPPEGVRLSPLAERQLQVQWEPPG
SWPFPEIFSLKYWIRYKRQGAARFHRVGPIEATSFILRAVRPRARYYVQVAAQDLTDYGELSDWSL
PATATMSLGK.

In embodiments, a chimeric protein comprises a variant of the extracellular domain of IL27B. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 82.

In embodiments, the second domain of a chimeric protein comprises an amino acid sequence that is at least 95% identical to the amino acid sequence of SEQ ID NO: 82.

One of ordinary skill may select variants of the known amino acid sequence of IL27B by consulting the literature, e.g., Iranshani et al., “Decreased Gene Expression of Epstein-Barr Virus-Induced Gene 3 (EBI-3) may Contribute to the Pathogenesis of Rheumatoid Arthritis,” Immunol. Invest. 48 (4), 367-377 (2019); Larousserie et al., “Expression of IL-27 in human Th1-associated granulomatous diseases,” J. Pathol. 202 (2), 164-171 (2004), each of which is incorporated by reference in its entirety.

In embodiments, a chimeric protein of the present disclosure comprises: (1) a first domain comprising the amino acid sequence of SEQ ID NO: 58, (b) a second domain comprises the amino acid sequence of SEQ ID NO: 82, and (c) a linker comprises an amino acid sequence that is at least 95% identical to SEQ ID NO: 1, SEQ ID NO: 2, SEQ ID NO: 3, or the linker underlined and/or in bold in SEQ ID NO: 83 below.

In embodiments, a DcR3-Beta-IL27B chimeric protein of the present disclosure has the following amino acid sequence:

(SEQ ID NO: 83)
AETPTYPWRDAETGERLVCAQCPPGTFVQRPCRRDSPTTCPCPPRHYTQFWNYLERCRYCNVL
CGEREEEARACHATHNRACCRTGFFAHAGFCLEHASCPPGAGVIAPGTPSQNTQCPCPPGTFSA
SSSSSEQCQPHRNCTALGLALNVPGSSSHDTLCGSGSDEGGEDGSKYGPPCPPCPAPEFLGGP
SVFLFPPKPKDQLMISRTPEVTCVVVDVSQEDPEVQFNWYVDGVEVHNAKTKPREEQFNSTYRV
VSVLTVLHQDWLSGKEYKCKVSSKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQEGNVFSCSVLHEAL
HNHYTQKSLSLSLGKRKCGKRGSGSRKGPPAALTLPRVQCRASRYPIAVDCSWTLPPAPNSTSPV
SFIATYRLGMAARGHSWPCLQQTPTSTSCTITDVQLFSMAPYVLNVTAVHPWGSSSSFVPFITEHII
KPDPPEGVRLSPLAERQLQVQWEPPGSWPFPEIFSLKYWIRYKRQGAARFHRVGPIEATSFILRAV
RPRARYYVQVAAQDLTDYGELSDWSLPATATMSLGK

In embodiments, a chimeric protein comprises a variant of a DcR3-Beta-IL27B chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 83.

### IL23R-Alpha-DcR3

In embodiments, the chimeric protein is capable of contemporaneously binding the IL23R ligand and the DcR3 ligand. In embodiments, the IL23R ligand is IL-23, and the DcR3 ligand is the DcR3 ligand is Fas ligand (FasL), LIGHT, or TL1A. Interleukin-23 receptor (IL23R) associates with IL12RB1 to form the interleukin-23 receptor. IL23R binds with IL23 to mediate Th17 T cell differentiation, NK cell activation and angiogenesis. IL23 is produced by innate immune cells and may participate in acute response to infection in peripheral tissues. IL23 may be responsible for autoimmune inflammatory diseases and be important for tumorigenesis. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Upregulation of DcR3 during inflammatory reactions exerts negative-feedback to suppress inflammation. Accordingly, a chimeric protein comprising the extracellular domains of IL23R and of DcR3 is capable of contemporaneously competitively inhibiting an immune activating signal (via IL23R and DcR3). In embodiments, this chimeric protein is referred to herein as IL23R-Alpha-DcR3.

In embodiments, the chimeric proteins of the present disclosure comprise variants of the extracellular domain, which includes the ligand-binding domain, of IL23R. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of IL23R, e.g., human IL23R.

In embodiments, the extracellular domain of IL23R has the following amino acid sequence:

(SEQ ID NO: 84)
GITNINCSGHIWVEPATIFKMGMNISIYCQAAIKNCQPRKLHFYKNGIKERFQITRINKTTARLWYKN
FLEPHASMYCTAECPKHFQETLICGKDISSGYPPDIPDEVTCVIYEYSGNMTCTWNAGKLTYIDTKY
WHVKSLETEEEQQYLTSSYINISTDSLQGGKKYLVWVQAANALGMEESKQLQIHLDDIVIPSAAVI
SRAETINATVPKTIIYWDSQTTIEKVSCEMRYKATTNQTWNVKEFDTNFTYVQQSEFYLEPNIKYVF
QVRCQETGKRYWQPWSSLFFHKTPETVPQVTSKAFQHDTWNSGLTVASISTGHLTSDNRGDIG.

In embodiments, a chimeric protein comprises a variant of the extracellular domain of IL23R. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 84.

In embodiments, the first domain of a chimeric protein comprises an amino acid sequence that is at least 95% identical to the amino acid sequence of SEQ ID NO: 84.

One of ordinary skill may select variants of the known amino acid sequence of IL23R by consulting the literature, e.g., Zou et al., “Associations between IL-23R gene polymorphisms and the susceptibility of rheumatoid arthritis: a meta-analysis,” Artif Cells Nanomed Biotechnol 47 (1), 951-956 (2019); Zakrzewski et al., “IL23R-Protective Coding Variant Promotes Beneficial Bacteria and Diversity in the Ileal Microbiome in Healthy Individuals Without Inflammatory Bowel Disease,” J Crohns Colitis 13 (4), 451-461 (2019); Kan et al, “Identification and characterization of multiple splice forms of the human interleukin-23 receptor alpha chain in mitogen-activated leukocytes,” Genes Immun. 9 (7), 631-639 (2008); Mancini et al, “A novel insertion variant of the human IL-23 receptor-alpha chain transcript,” Genes Immun. 9 (6), 566-569 (2008), each of which is incorporated by reference in its entirety.

In embodiments, the chimeric proteins of the present disclosure comprise the extracellular domain of DcR3 which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol, 65(4):657-67 (2003), each of which is incorporated by reference in its entirety.

In embodiments, a chimeric protein of the present disclosure comprises: (1) a first domain comprising the amino acid sequence of SEQ ID NO: 84, (b) a second domain comprises the amino acid sequence of SEQ ID NO: 58, and (c) a linker comprises an amino acid sequence that is at least 95% identical to SEQ ID NO: 1, SEQ ID NO: 2, SEQ ID NO: 3, or the linker underlined and/or in bold in SEQ ID NO: 85 below.

In embodiments, a IL23R-Alpha-DcR3 chimeric protein of the present disclosure has the following amino acid sequence:

(SEQ ID NO: 85)
GITNINCSGHIWVEPATIFKMGMNISIYCQAAIKNCQPRKLHFYKNGIKERFQITRINKTTARLWYKN
FLEPHASMYCTAECPKHFQETLICGKDISSGYPPDIPDEVTCVIYEYSGNMTCTWNAGKLTYIDTKY
WHVKSLETEEEQQYLTSSYINISTDSLQGGKKYLVWVQAANALGMEESKQLQIHLDDIVIPSAAVI
SRAETINATVPKTIIYWDSQTTIEKVSCEMRYKATTNQTWNVKEFDTNFTYVQQSEFYLEPNIKYVF
QVRCQETGKRYWQPWSSLFFHKTPETVPQVTSKAFQHDTWNSGLTVASISTGHLTSDNRGDIGG
SGSRKGGKRGSKYGPPCPPCPAPEFLGGPSVFLFPPKPKDQLMISRTPEVTCVVVDVSQEDPEV
QFNWYVDGVEVHNAKTKPREEQFNSTYRVVSVLTVLHQDWLSGKEYKCKVSSKGLPSSIEKTISK
AKGQPREPQVYTLPPSQEEMTKNQVSLTCLVKGFYPSDIAVEWESNGQPENNYKTTPPVLDSDG
SFFLYSRLTVDKSRWQEGNVFSCSVLHEALHNHYTQKSLSLSLGKDEGGEDGSGSRAETPTYPW
RDAETGERLVCAQCPPGTFVQRPCRRDSPTTCPCPPRHYTQFWNYLERCRYCNVLCGEREEEA
RACHATHNRACCRTGFFAHAGFCLEHASCPPGAGVIAPGTPSQNTQCPCPPGTFSASSSSSEQC
QPHRNCTALGLALNVPGSSSHDTLC.

In embodiments, a chimeric protein comprises a variant of a IL23R-Alpha-DcR3 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 85.

### IL12RB1-Beta-DcR3

In embodiments, the chimeric protein is capable of contemporaneously binding the IL12RB1 ligand and the DcR3 ligand. In embodiments, the IL12RB1 ligand is IL-12, and the DcR3 ligand is the DcR3 ligand is Fas ligand (FasL), LIGHT, or TL1A. Interleukin-12 receptor subunit beta 1 (IL12RB1) combines with IL23R to bind with interleukin-23 (IL23) which mediates Th17 T cell differentiation, NK cell activation and angiogenesis. IL23 is produced by innate immune cells and may participate in acute response to infection in peripheral tissues. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Upregulation of DcR3 during inflammatory reactions exerts negative-feedback to suppress inflammation. Accordingly, a chimeric protein comprising the extracellular domains of IL12RB1 and of DcR3 is capable of contemporaneously competitively inhibiting an immune activating signal (via IL12RB1 and DcR3). In embodiments, this chimeric protein is referred to herein as IL12RB1-Beta-DcR3.

In embodiments, the chimeric proteins of the present disclosure comprise variants of the extracellular domain, which includes the ligand-binding domain, of IL12RB1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of IL12RB1, e.g., human IL12RB1.

In embodiments, the extracellular domain of IL12RB1 has the following amino acid sequence:

(SEQ ID NO: 86)
CRTSECCFQDPPYPDADSGSASGPRDLRCYRISSDRYECSWQYEGPTAGVSHFLRCCLSSGRC
CYFAAGSATRLQFSDQAGVSVLYTVTLWVESWARNQTEKSPEVTLQLYNSVKYEPPLGDIKVSKL
AGQLRMEWETPDNQVGAEVQFRHRTPSSPWKLGDCGPQDDDTESCLCPLEMNVAQEFQLRRR
QLGSQGSSWSKWSSPVCVPPENPPQPQVRFSVEQLGQDGRRRLTLKEQPTQLELPEGCQGLAP
GTEVTYRLQLHMLSCPCKAKATRTLHLGKMPYLSGAAYNVAVISSNQFGPGLNQTWHIPADTHTE
PVALNISVGTNGTTMYWPARAQSMTYCIEWQPVGQDGGLATCSLTAPQDPDPAGMATYSWSRE
SGAMGQEKCYYITIFASAHPEKLTLWSTVLSTYHFGGNASAAGTPHHVSVKNHSLDSVSVDWAPS
LLSTCPGVLKEYVVRCRDEDSKQVSEHPVQPTETQVTLSGLRAGVAYTVQVRADTAWLRGVWSQ
PQRFSIEVQVSD.

In embodiments, a chimeric protein comprises a variant of the extracellular domain of IL12RB1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 86.

In embodiments, the first domain of a chimeric protein comprises an amino acid sequence that is at least 95% identical to the amino acid sequence of SEQ ID NO: 86.

One of ordinary skill may select variants of the known amino acid sequence of IL12RB1 by consulting the literature, e.g., Reeme et al., “Human IL12RB1 expression is allele-biased and produces a novel IL12 response regulator,” Genes Immun. 20 (3), 181-197 (2019); Song et al., “Associations of IL-12, IL12R polymorphisms and serum IL-12 levels with high-risk human papillomavirus susceptibility in rural women from Luohe, Henan, China,” Medicine (Baltimore) 98 (38), e16991 (2019); Rosain et al, “A Variety of Alu-Mediated Copy Number Variations Can Underlie IL-12Rbeta 1 Deficiency,” J. Clin. Immunol. 38 (5), 617-627 (2018); Presky et al, “A functional interleukin 12 receptor complex is composed of two beta-type cytokine receptor subunits,” Proc. Natl. Acad. Sci. U.S.A. 93 (24), 14002-14007 (1996); Gubler et al, “Molecular biology of interleukin-12 receptors,” Ann. N. Y. Acad. Sci. 795, 36-40 (1996), each of which is incorporated by reference in its entirety.

In embodiments, the chimeric proteins of the present disclosure comprise the extracellular domain of DcR3 which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol, 65(4):657-67 (2003), each of which is incorporated by reference in its entirety.

In embodiments, a chimeric protein of the present disclosure comprises: (1) a first domain comprising the amino acid sequence of SEQ ID NO: 86, (b) a second domain comprises the amino acid sequence of SEQ ID NO: 58, and (c) a linker comprises an amino acid sequence that is at least 95% identical to SEQ ID NO: 1, SEQ ID NO: 2, SEQ ID NO: 3, or the linker underlined and/or in bold in SEQ ID NO: 87 below.

In embodiments, a IL12RB1-Beta-DcR3 chimeric protein of the present disclosure has the following amino acid sequence:

(SEQ ID NO: 87)
CRTSECCFQDPPYPDADSGSASGPRDLRCYRISSDRYECSWQYEGPTAGVSHFLRCCLSSGRC
CYFAAGSATRLQFSDQAGVSVLYTVTLWVESWARNQTEKSPEVTLQLYNSVKYEPPLGDIKVSKL
AGQLRMEWETPDNQVGAEVQFRHRTPSSPWKLGDCGPQDDDTESCLCPLEMNVAQEFQLRRR
QLGSQGSSWSKWSSPVCVPPENPPQPQVRFSVEQLGQDGRRRLTLKEQPTQLELPEGCQGLAP
GTEVTYRLQLHMLSCPCKAKATRTLHLGKMPYLSGAAYNVAVISSNQFGPGLNQTWHIPADTHTE
PVALNISVGTNGTTMYWPARAQSMTYCIEWQPVGQDGGLATCSLTAPQDPDPAGMATYSWSRE
SGAMGQEKCYYITIFASAHPEKLTLWSTVLSTYHFGGNASAAGTPHHVSVKNHSLDSVSVDWAPS
LLSTCPGVLKEYWVRCRDEDSKQVSEHPVQPTETQVTLSGLRAGVAYTVQVRADTAWLRGVWSQ
PQRFSIEVQVSDGSGSDEGGEDGSKYGPPCPPCPAPEFLGGPSVFLFPPKPKDQLMISRTPEVTC
WVVDVSQEDPEVQFNWYVDGVEVHNAKTKPREEQFNSTYRVVSVLTVLHQDWLSGKEYKCKVS
SKGLPSSIEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVLHEALHNHYTQKSLSLSLGKRKGGKR
GSGSRAETPTYPWRDAETGERLVCAQCPPGTFVQRPCRRDSPTTCPCPPRHYTQFWNYLERCR
YCNVLCGEREEEARACHATHNRACCRTGFFAHAGFCLEHASCPPGAGVIAPGTPSQNTQCPCPP
GTFSASSSSSEQCQPHRNCTALGLALNVPGSSSHDTLC.

In embodiments, a chimeric protein comprises a variant of a IL12RB1-Beta-DcR3 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 87.

### ITGA4-Alpha-DcR3

In embodiments, the chimeric protein is capable of contemporaneously binding the ITGA4 ligand and the DcR3 ligand. In embodiments, the ITGA4 ligand, in combination with ITGB7, is MADCAM, and the DcR3 ligand is the DcR3 ligand is Fas ligand (FasL), LIGHT, or TL1A. Integrin alpha 4 subunit (ITGA4), in combination with Integrin beta 7 (ITGB7), binds with MADCAM. Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Upregulation of DcR3 during inflammatory reactions exerts negative-feedback to suppress inflammation. Accordingly, without wishing to be bound by theory, a chimeric protein comprising the extracellular domains of ITGA4 and of DcR3 is capable of contemporaneously competitively inhibiting entry to the gut mucosal compartment (via ITGA4) and by competitively inhibiting an integrin that facilitates attachment and migration of an immune cell across an endothelial surface (DcR3). In embodiments, this chimeric protein is referred to herein as ITGA4-Alpha-DcR3.

In embodiments, the chimeric proteins of the present disclosure comprise variants of the extracellular domain, which includes the ligand-binding domain, of ITGA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of ITGA4, e.g., human ITGA4.

In embodiments, the extracellular domain of ITGA4 has the following amino acid sequence:

(SEQ ID NO: 88)
YNVDTESALLYQGPHNTLFGYSVVLHSHGANRWLLVGAPTANWLANASVINPGAIYRCRIGKNPG
QTCEQLQLGSPNGEPCGKTCLEERDNQWLGVTLSRQPGENGSIVTCGHRWKNIFYIKNENKLPT
GGCYGVPPDLRTELSKRIAPCYQDYVKKFGENFASCQAGISSFYTKDLIVMGAPGSSYWTGSLFV
YNITTNKYKAFLDKQNQVKFGSYLGYSVGAGHFRSQHTTEVVGGAPQHEQIGKAYIFSIDEKELNIL
HEMKGKKLGSYFGASVCAVDLNADGFSDLLVGAPMQSTIREEGRVFVYINSGSGAVMNAMETNL
VGSDKYAARFGESIVNLGDIDNDGFEDVAIGAPQEDDLQGAIYIYNGRADGISSTFSQRIEGLQISKS
LSMFGQSISGQIDADNNGYVDVAVGAFRSDSAVLLRTR.

In embodiments, a chimeric protein comprises a variant of the extracellular domain of ITGA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 88.

In embodiments, the first domain of a chimeric protein comprises an amino acid sequence that is at least 95% identical to the amino acid sequence of SEQ ID NO: 88.

One of ordinary skill may select variants of the known amino acid sequence of ITGA4 by consulting the literature, e.g., Takada et al., “The primary structure of the alpha 4 subunit of VLA-4: homology to other integrins and a possible cell-cell adhesion function,” EMBO J. 8 (5), 1361-1368 (1989); Rosen et al., “Characterization of the alpha 4 integrin gene promoter,” Proc. Natl. Acad. Sci. U.S.A. 88 (10), 4094-4098 (1991); Szabo et al, “Identification of two variants of the human integrin alpha 4 subunit,” Mol. Immunol. 32 (17-18), 1453-1454 (1995), each of which is incorporated by reference in its entirety.

In embodiments, the chimeric proteins of the present disclosure comprise the extracellular domain of DcR3 which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol, 65(4):657-67 (2003), each of which is incorporated by reference in its entirety.

In embodiments, a chimeric protein of the present disclosure comprises: (1) a first domain comprising the amino acid sequence of SEQ ID NO: 88, (b) a second domain comprises the amino acid sequence of SEQ ID NO: 58, and (c) a linker comprises an amino acid sequence that is at least 95% identical to SEQ ID NO: 1, SEQ ID NO: 2, SEQ ID NO: 3, or the linker underlined and/or in bold in SEQ ID NO: 89 below.

In embodiments, a ITGA4-Alpha-DcR3 chimeric protein of the present disclosure has the following amino acid sequence:

(SEQ ID NO: 89)
YNVDTESALLYQGPHNTLFGYSVVLHSHGANRWLLVGAPTANWLANASVINPGAIYRCRIGKNPG
QTCEQLQLGSPNGEPCGKTCLEERDNQWLGVTLSRQPGENGSIVTCGHRWKNIFYIKNENKLPT
GGCYGVPPDLRTELSKRIAPCYQDYVKKFGENFASCQAGISSFYTKDLIVMGAPGSSYWTGSLFV
YNITTNKYKAFLDKQNQVKFGSYLGYSVGAGHFRSQHTTEVVGGAPQHEQIGKAYIFSIDEKELNIL
HEMKGKKLGSYFGASVCAVDLNADGFSDLLVGAPMQSTIREEGRVFVYINSGSGAVMNAMETNL
VGSDKYAARFGESIVNLGDIDNDGFEDVAIGAPQEDDLQGAIYIYNGRADGISSTFSQRIEGLQISKS
LSMFGQSISGQIDADNNGYVDVAVGAFRSDSAVLLRTRGSGSRKGGKRGSKYGPPCPPCPAPEF
LGGPSVFLFPPKPKDQLMISRTPEVTCVVVDVSQEDPEVQFNWYVDGVEVHNAKTKPREEQFNS
TYRWVSVLTVLHQDWLSGKEYKCKVSSKGLPSSIEKTISKAKGQPREPQVYTLPPSQEEMTKNQV
SLTCLVKGFYPSDIAVEWESNGQPENNYKTTPPVLDSDGSFFLYSRLTVDKSRWQEGNVFSCSVL
HEALHNHYTQKSLSLSLGKDEGGEDGSGSAETPTYPWRDAETGERLVCAQCPPGTFVQRPCRR
DSPTTCPCPPRHYTQFWNYLERCRYCNVLCGEREEEARACHATHNRACCRTGFFAHAGFCLEH
ASCPPGAGVIAPGTPSQNTQCPCPPGTFSASSSSSEQCQPHRNCTALGLALNVPGSSSHDTLC.

In embodiments, a chimeric protein comprises a variant of a ITGA4-Alpha-DcR3 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 89.

### ITGB7-Beta-DcR3

In embodiments, the chimeric protein is capable of contemporaneously binding the ITGB7 ligand and the DcR3 ligand. In embodiments, the ITGB7 ligand, once paired with ITGA4, is MADCAM, and the DcR3 ligand is the DcR3 ligand is Fas ligand (FasL), LIGHT, or TL1A. Integrin beta 7 subunit (ITGB7), paired with ITGA4, binds with MADCAM Decoy receptor 3 (DcR3), also known as tumor necrosis factor receptor (TNFR) superfamily member 6b (TNFRSF6B), is a soluble decoy receptor which can neutralize the biological functions of three members of tumor necrosis factor superfamily (TNFSF): Fas ligand (FasL), LIGHT, and TL1A. In addition to ‘decoy’ function, recombinant DcR3 is able to modulate the activation and differentiation of dendritic cells (DCs) and macrophages via ‘non-decoy’ action. Upregulation of DcR3 during inflammatory reactions exerts negative-feedback to suppress inflammation. Accordingly, without wishing to be bound by theory, a chimeric protein comprising the extracellular domains of ITGB7 and of DcR3 is capable of contemporaneously competitively inhibiting entry to the gut mucosal compartment (via ITGB7) and by competitively inhibiting an immune activating signal (DcR3). In embodiments, this chimeric protein is referred to herein as ITGB7-Beta-DcR3.

In embodiments, the chimeric proteins of the present disclosure comprise variants of the extracellular domain, which includes the ligand-binding domain, of ITGB7. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of ITGB7, e.g., human ITGB7.

In embodiments, the extracellular domain of ITGB7 has the following amino acid sequence:

(SEQ ID NO: 90)
MVALPMVLVLLLVLSRGESELDAKIPSTGDATEWRNPHLSMLGSCQPAPSCQKCILSHPSCAWCK
QLNFTASGEAEARRCARREELLARGCPLEELEEPRGQQEVLQDQPLSQGARGEGATQLAPQRVR
VTLRPGEPQQLQVRFLRAEGYPVDLYYLMDLSYSMKDALERVRQLGHALLVRLQEVTHSVRIGFG
SFVDKTVLPFVSTVPSKLRHPCPTRLERCQSPFSFHHVLSLTGDAQAFEREVGRQSVSGNLDSPE
GGFDAILQAALCQEQIGWRNVSRLLVFTSDDTFHTAGDGKLGGIFMPSDGHCHLDSNGLYSRSTE
FDYPSVGQVAQALSAANIQPIFAVTSAALPVYQELSKLIPKSAVGELSEDSSNVVQLIMDAYNSLSS
TVTLEHSSLPPGVHISYESQCEGPEKREGKAEDRGQCNHVRINQTVTFWVSLQATHCLPEPHLLR
LRALGFSEELIVELHTLC.

In embodiments, a chimeric protein comprises a variant of the extracellular domain of ITGB7. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 90.

In embodiments, the first domain of a chimeric protein comprises an amino acid sequence that is at least 95% identical to the amino acid sequence of SEQ ID NO: 90.

One of ordinary skill may select variants of the known amino acid sequence of ITGB7 by consulting the literature, e.g., Sun et al., “Transmission of integrin beta 7 transmembrane domain topology enables gut lymphoid tissue development,” J. Cell Biol. 217 (4), 1453-1465 (2018); Erle et al., “Lung epithelial lining fluid T cell subsets defined by distinct patterns of beta 7 and beta 1 integrin expression,” Am. J. Respir. Cell Mol. Biol. 10 (3), 237-244 (1994); Jiang et al, “The gene organization of the human beta 7 subunit, the common beta subunit of the leukocyte integrins HML-1 and LPAM-1,” Int. Immunol. 4 (9), 1031-1040 (1992); Erle et al, “Complete amino acid sequence of an integrin beta subunit (beta 7) identified in leukocytes,” J. Biol. Chem. 266 (17), 11009-11016 (1991), each of which is incorporated by reference in its entirety.

In embodiments, the chimeric proteins of the present disclosure comprise the extracellular domain of DcR5 which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of DcR3, e.g., human DcR3.

In embodiments, the extracellular domain of DcR3 has the amino acid sequence of SEQ ID NO: 58.

In embodiments, a chimeric protein comprises a variant of the extracellular domain of DcR3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at 25 least about 97%, or at least about 98%, or at least about 99% sequence identity with SEQ ID NO: 58.

In embodiments, the second domain of a chimeric protein comprises an amino acid sequence that is at least 95% identical to the amino acid sequence of SEQ ID NO: 58.

One of ordinary skill may select variants of the known amino acid sequence of DcR3 by consulting the literature, e.g., Soliman et al., “Association of Tumor Necrosis Like factor 1 A (TL1A) and its Decoy Receptor (DcR3) with The Disease Activity and Autoantibody Production in Rheumatoid Arthritis Patients,” Egypt J Immunol 26 (1), 43-54 (2019); Bou-Dargham et al., “Subgrouping breast cancer patients based on immune evasion mechanisms unravels a high involvement of transforming growth factor-beta and decoy receptor 3,” PLoS ONE 13 (12), e0207799 (2018); Xie et al., “Effects of miR-340 on hepatocellular carcinoma by targeting the DcR3 gene,” Dig Liver Dis 50 (3), 291-296 (2018); Hsieh et al., “Decoy receptor 3: an endogenous immunomodulator in cancer growth and inflammatory reactions.” J. Biomed. Sci. 24 (39), 1-9 (2017); Cardinale et al., “Targeted resequencing identifies defective variants of decoy receptor 3 in pediatric-onset inflammatory bowel disease.” Genes & Immunity volume 14, pages 447-452(2013); and Wroblewski et al., “Decoy receptor 3 (DcR3) is proteolytically processed to a metabolic fragment having differential activities against Fas ligand and LIGHT.” Biochem Pharmacol, 65(4):657-67 (2003), each of which is incorporated by reference in its entirety.

In embodiments, a chimeric protein of the present disclosure comprises: (1) a first domain comprising the amino acid sequence of SEQ ID NO: 90, (b) a second domain comprises the amino acid sequence of SEQ ID NO: 58, and (c) a linker comprises an amino acid sequence that is at least 95% identical to SEQ ID NO: 1, SEQ ID NO: 2, SEQ ID NO: 3, or the linker underlined and/or in bold in SEQ ID NO: 91 below.

In embodiments, a ITGB7-Beta-DcR3 chimeric protein of the present disclosure has the amino acid sequence:

(SEQ ID NO: 91)
MVALPMVLVLLLVLSRGESELDAKIPSTGDATEWRNPHLSMLGSCQPAPSCQKCILSHPSCAWCK
QLNFTASGEAEARRCARREELLARGCPLEELEEPRGQQEVLQDQPLSQGARGEGATQLAPQRVR
VTLRPGEPQQLQVRFLRAEGYPVDLYYLMDLSYSMKDALERVRQLGHALLVRLQEVTHSVRIGFG
SFVDKTVLPFVSTVPSKLRHPCPTRLERCQSPFSFHHVLSLTGDAQAFEREVGRQSVSGNLDSPE
GGFDAILQAALCQEQIGWRNVSRLLVFTSDDTFHTAGDGKLGGIFMPSDGHCHLDSNGLYSRSTE
FDYPSVGQVAQALSAANIQPIFAVTSAALPVYQELSKLIPKSAVGELSEDSSNVVQLIMDAYNSLSS
TVTLEHSSLPPGVHISYESQCEGPEKREGKAEDRGQCNHVRINQTVTFWVSLQATHCLPEPHLLR
LRALGFSEELIVELHTLCGSGSDEGGEDGSKYGPPCPPCPAPEFLGGPSVFLFPPKPKDQLMISRT
PEVTCVVVDVSQEDPEVQFNWYVDGVEVHNAKTKPREEQFNSTYRVVSVLTVLHQDWLSGKEYK
CKVSSKGLPSSIEKTISKAKGQPREPQVYTLPPSQEEMTKNQVSLTCLVKGFYPSDIAVEWESNGQ
PENNYKTTPPVLDSDGSFFLYSRLTVDKSRWQEGNVFSCSVLHEALHNHYTQKSLSLSLGKRKGG
KRGSGSAETPTYPWRDAETGERLVCAQCPPGTFVQRPCRRDSPTTCPCPPRHYTQFWNYLERC
RYCNVLCGEREEEARACHATHNRACCRTGFFAHAGFCLEHASCPPGAGVIAPGTPSQNTQCPCP
PGTFSASSSSSEQCQPHRNCTALGLALNVPGSSSHDTLC.

In embodiments, a chimeric protein comprises a variant of a ITGB7-Beta-DcR3 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 91.

### ITGA4-Alpha-GITRL

In embodiments, the chimeric protein is capable of contemporaneously binding the ITGA4 ligand and the GITRL receptor. In embodiments, the ITGA4 ligand, in combination with ITGB7, is MADCAM, and the GITRL receptor is GITR. Integrin alpha 4 subunit (ITGA4), in combination with ITGB7, binds with MADCAM. Glucocorticoid-induced TNFR-related protein ligand (GITRL) binds with its receptor GITR and functions as a co-activating signal for the development of the immune system by influencing the activity of effector and regulatory T cells. Accordingly, without wishing to be bound by theory, a chimeric protein comprising the extracellular domains of ITGA4 and of GITRL is capable of contemporaneously competitively inhibiting an immune activating signal (via GITRL). In embodiments, this chimeric protein is referred to herein as ITGA4-Alpha-GITRL.

In embodiments, the chimeric proteins of the present disclosure comprise variants of the extracellular domain, which includes the ligand-binding domain, of ITGA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of ITGA4, e.g., human ITGA4.

In embodiments, the extracellular domain of ITGA4 has the following amino acid sequence:

(SEQ ID NO: 88)
YNVDTESALLYQGPHNTLFGYSVVLHSHGANRWLLVGAPTANWLANASVI
NPGAIYRCRIGKNPGQTCEQLQLGSPNGEPCGKTCLEERDNQWLGVTLSR
QPGENGSIVTCGHRWKNIFYIKNENKLPTGGCYGVPPDLRTELSKRIAPC
YQDYVKKFGENFASCQAGISSFYTKDLIVMGAPGSSYWTGSLFVYNITTN
KYKAFLDKQNQVKFGSYLGYSVGAGHFRSQHTTEVVGGAPQHEQIGKAYI
FSIDEKELNILHEMKGKKLGSYFGASVCAVDLNADGFSDLLVGAPMQSTI
REEGRVFVYINSGSGAVMNAMETNLVGSDKYAARFGESIVNLGDIDNDGF
EDVAIGAPQEDDLQGAIYIYNGRADGISSTFSQRIEGLQISKSLSMFGQS
ISGQIDADNNGYVDVAVGAFRSDSAVLLRTR.

In embodiments, a chimeric protein comprises a variant of the extracellular domain of ITGA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 88.

In embodiments, the first domain of a chimeric protein comprises an amino acid sequence that is at least 95% identical to the amino acid sequence of SEQ ID NO: 88.

One of ordinary skill may select variants of the known amino acid sequence of ITGA4 by consulting the literature, e.g., Takada et al., “The primary structure of the alpha 4 subunit of VLA-4: homology to other integrins and a possible cell-cell adhesion function,” EMBO J. 8 (5), 1361-1368 (1989); Rosen et al., “Characterization of the alpha 4 integrin gene promoter,” Proc. Natl. Acad. Sci. U.S.A. 88 (10), 4094-4098 (1991); Szabo et al, “Identification of two variants of the human integrin alpha 4 subunit,” Mol. Immunol. 32 (17-18), 1453-1454 (1995), each of which is incorporated by reference in its entirety.

In embodiments, the chimeric proteins of the present disclosure comprise the extracellular domain of GITRL which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of GITRL, e.g., human GITRL.

In embodiments, the extracellular domain of GITRL has the following amino acid sequence:

(SEQ ID NO: 92)
QLETAKEPCMAKFGPLPSKWQMASSEPPCVNKVSDWKLEILQNGLYLIYG
QVAPNANYNDVAPFEVRLYKNKDMIQTLTNKSKIQNVGGTYELHVGDTID
LIFNSEHQVLKNNTYWGIILLANPQFIS.

In embodiments, a chimeric protein comprises a variant of the extracellular domain of GITRL. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at 25 least about 97%, or at least about 98%, or at least about 99% sequence identity with SEQ ID NO: 92.

In embodiments, the second domain of a chimeric protein comprises an amino acid sequence that is at least 95% identical to the amino acid sequence of SEQ ID NO: 92.

One of ordinary skill may select variants of the known amino acid sequence of GITRL by consulting the literature, e.g., Gurney et al., “Identification of a new member of the tumor necrosis factor family and its receptor, a human ortholog of mouse GITR,” Curr. Biol. 9 (4), 215-218 (1999); Li et al., “GITRL is associated with increased autoantibody production in patients with rheumatoid arthritis,” Clin. Rheumatol. 35 (9), 2195-2202 (2016); Tang et al, “GITRL modulates the activities of p38 MAPK and STAT3 to promote Th17 cell differentiation in autoimmune arthritis,” Oncotarget 7 (8), 8590-8600 (2016), each of which is incorporated by reference in its entirety.

In embodiments, a chimeric protein of the present disclosure comprises: (1) a first domain comprising the amino acid sequence of SEQ ID NO: 88, (b) a second domain comprises the amino acid sequence of SEQ ID NO: 92, and (c) a linker comprises an amino acid sequence that is at least 95% identical to SEQ ID NO: 1, SEQ ID NO: 2, SEQ ID NO: 3, or the linker underlined and/or in bold in SEQ ID NO: 93 below.

In embodiments, a ITGA4-Alpha-GITRL chimeric protein of the present disclosure has the following amino acid sequence:

(SEQ ID NO: 93)
YNVDTESALLYQGPHNTLFGYSVVLHSHGANRWLLVGAPTANWLANASVI
NPGAIYRCRIGKNPGQTCEQLQLGSPNGEPCGKTCLEERDNQWLGVTLSR
QPGENGSIVTCGHRWKNIFYIKNENKLPTGGCYGVPPDLRTELSKRIAPC
YQDYVKKFGENFASCQAGISSFYTKDLIVMGAPGSSYWTGSLFVYNITTN
KYKAFLDKQNQVKFGSYLGYSVGAGHFRSQHTTEVVGGAPQHEQIGKAYI
FSIDEKELNILHEMKGKKLGSYFGASVCAVDLNADGFSDLLVGAPMQSTI
REEGRVFVYINSGSGAVMNAMETNLVGSDKYAARFGESIVNLGDIDNDGF
EDVAIGAPQEDDLQGAIYIYNGRADGISSTFSQRIEGLQISKSLSMFGQS
ISGQIDADNNGYVDVAVGAFRSDSAVLLRTRGSGSRKGGKRGSKYGPPCP
PCPAPEFLGGPSVFLFPPKPKDQLMISRTPEVTCVVVDVSQEDPEVQFNW
YVDGVEVHNAKTKPREEQFNSTYRVVSVLTVLHQDWLSGKEYKCKVSSKG
LPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSV
LHEALHNHYTQKSLSLSLGKDEGGEDGSGSQLETAKEPCMAKFGPLPSKW
QMASSEPPCVNKVSDWKLEILQNGLYLIYGQVAPNANYNDVAPFEVRLYK
NKDMIQTLTNKSKIQNVGGTYELHVGDTIDLIFNSEHQVLKNNTYWGIIL
LANPQFIS.

In embodiments, a chimeric protein comprises a variant of a ITGA4-Alpha-GITRL chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 93.

### ITGB7-Beta-GITRL

In embodiments, the chimeric protein is capable of contemporaneously binding the ITGB7 ligand and the GITRL receptor. In embodiments, the ITGB7 ligand, in combination with ITGA4, is MADCAM, and the GITRL receptor is GITR. Integrin beta 7 subunit (ITGB7), once dimerized with integrin alpha 4 subunit (ITGA4), binds with MADCAM. Glucocorticoid-induced TNFR-related protein ligand (GITRL) binds with its receptor GITR and functions as a co-activating signal for the development of the immune system by influencing the activity of effector and regulatory T cells. Accordingly, without wishing to be bound by theory, a chimeric protein comprising the extracellular domains of ITGB7 and of GITRL is capable of contemporaneously competitively inhibiting entry to the gut mucosal compartment (via ITGB7) and by competitively inhibiting an immune activating signal (via GITRL). In embodiments, this chimeric protein is referred to herein as ITGB7-Beta-GITRL.

In embodiments, the chimeric proteins of the present disclosure comprise variants of the extracellular domain, which includes the ligand-binding domain, of ITGB7. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of ITGB7, e.g., human ITGB7.

In embodiments, the extracellular domain of ITGB7 has the following amino acid sequence:

(SEQ ID NO: 90)
MVALPMVLVLLLVLSRGESELDAKIPSTGDATEWRNPHLSMLGSCQPAPS
CQKCILSHPSCAWCKQLNFTASGEAEARRCARREELLARGCPLEELEEPR
GQQEVLQDQPLSQGARGEGATQLAPQRVRVTLRPGEPQQLQVRFLRAEGY
PVDLYYLMDLSYSMKDALERVRQLGHALLVRLQEVTHSVRIGFGSFVDKT
VLPFVSTVPSKLRHPCPTRLERCQSPFSFHHVLSLTGDAQAFEREVGRQS
VSGNLDSPEGGFDAILQAALCQEQIGWRNVSRLLVFTSDDTFHTAGDGKL
GGIFMPSDGHCHLDSNGLYSRSTEFDYPSVGQVAQALSAANIQPIFAVTS
AALPVYQELSKLIPKSAVGELSEDSSNVVQLIMDAYNSLSSTVTLEHSSL
PPGVHISYESQCEGPEKREGKAEDRGQCNHVRINQTVTFWVSLQATHCLP
EPHLLRLRALGFSEELIVELHTLC.

In embodiments, a chimeric protein comprises a variant of the extracellular domain of ITGB7. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 90.

In embodiments, the first domain of a chimeric protein comprises an amino acid sequence that is at least 95% identical to the amino acid sequence of SEQ ID NO: 90.

One of ordinary skill may select variants of the known amino acid sequence of ITGB7 by consulting the literature, e.g., Sun et al., “Transmission of integrin beta 7 transmembrane domain topology enables gut lymphoid tissue development,” J. Cell Biol. 217 (4), 1453-1465 (2018); Erle et al., “Lung epithelial lining fluid T cell subsets defined by distinct patterns of beta 7 and beta 1 integrin expression,” Am. J. Respir. Cell Mol. Biol. 10 (3), 237-244 (1994); Jiang et al, “The gene organization of the human beta 7 subunit, the common beta subunit of the leukocyte integrins HML-1 and LPAM-1,” Int. Immunol. 4 (9), 1031-1040 (1992); Erle et 25 al, “Complete amino acid sequence of an integrin beta subunit (beta 7) identified in leukocytes,” J. Biol. Chem. 266 (17), 11009-11016 (1991), each of which is incorporated by reference in its entirety.

In embodiments, the chimeric proteins of the present disclosure comprise the extracellular domain of GITRL which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of GITRL, e.g., human GITRL.

In embodiments, the extracellular domain of GITRL has the following amino acid sequence:

(SEQ ID NO: 92)
QLETAKEPCMAKFGPLPSKWQMASSEPPCVNKVSDWKLEILQNGLYLIYG
QVAPNANYNDVAPFEVRLYKNKDMIQTLTNKSKIQNVGGTYELHVGDTID
LIFNSEHQVLKNNTYWGIILLANPQFIS.

In embodiments, a chimeric protein comprises a variant of the extracellular domain of GITRL. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 92.

In embodiments, the second domain of a chimeric protein comprises an amino acid sequence that is at least 95% identical to the amino acid sequence of SEQ ID NO: 92.

One of ordinary skill may select variants of the known amino acid sequence of GITRL by consulting the literature, e.g., Gurney et al., “Identification of a new member of the tumor necrosis factor family and its receptor, a human ortholog of mouse GITR,” Curr. Biol. 9 (4), 215-218 (1999); Li et al., “GITRL is associated with increased autoantibody production in patients with rheumatoid arthritis,” Clin. Rheumatol. 35 (9), 2195-2202 (2016); Tang et al, “GITRL modulates the activities of p38 MAPK and STAT3 to promote Th17 cell differentiation in autoimmune arthritis,” Oncotarget 7 (8), 8590-8600 (2016), each of which is incorporated by reference in its entirety.

In embodiments, a chimeric protein of the present disclosure comprises: (1) a first domain comprising the amino acid sequence of SEQ ID NO: 90, (b) a second domain comprises the amino acid sequence of SEQ ID NO: 92, and (c) a linker comprises an amino acid sequence that is at least 95% identical to SEQ ID NO: 1, SEQ ID NO: 2, SEQ ID NO: 3, or the linker underlined and/or in bold in SEQ ID NO: 94 below.

In embodiments, a ITGB7-Beta-GITRL chimeric protein of the present disclosure has the following amino acid sequence:

(SEQ ID NO: 94)
MVALPMVLVLLLVLSRGESELDAKIPSTGDATEWRNPHLSMLGSCQPAPS
CQKCILSHPSCAWCKQLNFTASGEAEARRCARREELLARGCPLEELEEPR
GQQEVLQDQPLSQGARGEGATQLAPQRVRVTLRPGEPQQLQVRFLRAEGY
PVDLYYLMDLSYSMKDALERVRQLGHALLVRLQEVTHSVRIGFGSFVDKT
VLPFVSTVPSKLRHPCPTRLERCQSPFSFHHVLSLTGDAQAFEREVGRQS
VSGNLDSPEGGFDAILQAALCQEQIGWRNVSRLLVFTSDDTFHTAGDGKL
GGIFMPSDGHCHLDSNGLYSRSTEFDYPSVGQVAQALSAANIQPIFAVTS
AALPVYQELSKLIPKSAVGELSEDSSNVVQLIMDAYNSLSSTVTLEHSSL
PPGVHISYESQCEGPEKREGKAEDRGQCNHVRINQTVTFWVSLQATHCLP
EPHLLRLRALGFSEELIVELHTLCGSGSDEGGEDGSKYGPPCPPCPAPEF
LGGPSVFLFPPKPKDQLMISRTPEVTCVVVDVSQEDPEVQFNWYVDGVEV
HNAKTKPREEQFNSTYRVVSVLTVLHQDWLSGKEYKCKVSSKGLPSSIEK
TISKAKGQPREPQVYTLPPSQEEMTKNQVSLTCLVKGFYPSDIAVEWESN
GQPENNYKTTPPVLDSDGSFFLYSRLTVDKSRWQEGNVFSCSVLHEALHN
HYTQKSLSLSLGKRKGGKRGSGSQLETAKEPCMAKFGPLPSKWQMASSEP
PCVNKVSDWKLEILQNGLYLIYGQVAPNANYNDVAPFEVRLYKNKDMIQT
LTNKSKIQNVGGTYELHVGDTIDLIFNSEHQVLKNNTYWGIILLANPQFI
S.

In embodiments, a chimeric protein comprises a variant of a ITGB7-Beta-GITRL chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 94.

### ITGA4-Alpha-IL10

In embodiments, the chimeric protein is capable of contemporaneously binding the ITGA4 ligand and the IL10 receptor. In embodiments, the ITGA4 ligand, in combination with ITGB7, is MADCAM, and the IL10 receptor is IL10R. Integrin alpha 4 subunit (ITGA4) in combination with ITGB7, binds with MADCAM. Interleukin-10 binds with its receptor Interleukin-10 receptor (IL10R) and functions as a key anti-inflammatory cytokine that can inhibit proinflammatory responses of both innate and adaptive immune cells, particularly in the intestinal mucosa. Accordingly, without wishing to be bound by theory, a chimeric protein comprising the extracellular domains of ITGA4 and IL10 is capable of contemporaneously preventing the entry of potentially pathogentic lymphocytes to the intestinal mucosa (via ITGA4) and activating an immune inhibitory signal (via IL10). In embodiments, this chimeric protein is referred to herein as ITGA4-Alpha-IL10.

In embodiments, the chimeric proteins of the present disclosure comprise variants of the extracellular domain, which includes the ligand-binding domain, of ITGA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of ITGA4, e.g., human ITGA4.

In embodiments, the extracellular domain of ITGA4 has the following amino acid sequence:

(SEQ ID NO: 88)
YNVDTESALLYQGPHNTLFGYSVVLHSHGANRWLLVGAPTANWLANASVI
NPGAIYRCRIGKNPGQTCEQLQLGSPNGEPCGKTCLEERDNQWLGVTLSR
QPGENGSIVTCGHRWKNIFYIKNENKLPTGGCYGVPPDLRTELSKRIAPC
YQDYVKKFGENFASCQAGISSFYTKDLIVMGAPGSSYWTGSLFVYNITTN
KYKAFLDKQNQVKFGSYLGYSVGAGHFRSQHTTEVVGGAPQHEQIGKAYI
FSIDEKELNILHEMKGKKLGSYFGASVCAVDLNADGFSDLLVGAPMQSTI
REEGRVFVYINSGSGAVMNAMETNLVGSDKYAARFGESIVNLGDIDNDGF
EDVAIGAPQEDDLQGAIYIYNGRADGISSTFSQRIEGLQISKSLSMFGQS
ISGQIDADNNGYVDVAVGAFRSDSAVLLRTR.

In embodiments, a chimeric protein comprises a variant of the extracellular domain of ITGA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 88.

In embodiments, the first domain of a chimeric protein comprises an amino acid sequence that is at least 95% identical to the amino acid sequence of SEQ ID NO: 88.

One of ordinary skill may select variants of the known amino acid sequence of ITGA4 by consulting the literature, e.g., Takada et al., “The primary structure of the alpha 4 subunit of VLA-4: homology to other integrins and a possible cell-cell adhesion function,” EMBO J. 8 (5), 1361-1368 (1989); Rosen et al., “Characterization of the alpha 4 integrin gene promoter,” Proc. Natl. Acad. Sci. U.S.A. 88 (10), 4094-4098 (1991); Szabo et al, “Identification of two variants of the human integrin alpha 4 subunit,” Mol. Immunol. 32 (17-18), 1453-1454 (1995), each of which is incorporated by reference in its entirety.

In embodiments, the chimeric proteins of the present disclosure comprise the extracellular domain of IL10 which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of IL10, e.g., human IL10.

In embodiments, the extracellular domain of IL10 has the following amino acid sequence:

(SEQ ID NO: 95)
SPGQGTQSENSCTHFPGNLPNMLRDLRDAFSRVKTFFQMKDQLDNLLLKE
SLLEDFKGYLGCQALSEMIQFYLEEVMPQAENQDPDIKAHVNSLGENLKT
LRLRLRRCHRFLPCENKSKAVEQVKNAFNKLQEKGIYKAMSEFDIFINYI
EAYMTMKIRN.

In embodiments, a chimeric protein comprises a variant of the extracellular domain of IL10. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 95.

In embodiments, the second domain of a chimeric protein comprises an amino acid sequence that is at least 95% identical to the amino acid sequence of SEQ ID NO: 95.

One of ordinary skill may select variants of the known amino acid sequence of IL10 by consulting the literature, e.g., Cook et al., “Crystallization and preliminary X-ray investigation of recombinant human interleukin 10,” Proteins 22 (2), 187-190 (1995); Walter et al., “Crystal structure of interleukin 10 reveals an interferon gamma-like fold,” Biochemistry 34 (38), 12118-12125 (1995), each of which is incorporated by reference in its entirety.

In embodiments, a chimeric protein of the present disclosure comprises: (1) a first domain comprising the amino acid sequence of SEQ ID NO: 88, (b) a second domain comprises the amino acid sequence of SEQ ID NO: 95, and (c) a linker comprises an amino acid sequence that is at least 95% identical to SEQ ID NO: 1, SEQ ID NO: 2, SEQ ID NO: 3, or the linker underlined and/or in bold in SEQ ID NO: 96 below.

In embodiments, a ITGA4-Alpha-IL10 chimeric protein of the present disclosure has the following amino acid sequence:

(SEQ ID NO: 96)
YNVDTESALLYQGPHNTLFGYSVVLHSHGANRWLLVGAPTANWLANASVI
NPGAIYRCRIGKNPGQTCEQLQLGSPNGEPCGKTCLEERDNQWLGVTLSR
QPGENGSIVTCGHRWKNIFYIKNENKLPTGGCYGVPPDLRTELSKRIAPC
YQDYVKKFGENFASCQAGISSFYTKDLIVMGAPGSSYWTGSLFVYNITTN
KYKAFLDKQNQVKFGSYLGYSVGAGHFRSQHTTEVVGGAPQHEQIGKAYI
FSIDEKELNILHEMKGKKLGSYFGASVCAVDLNADGFSDLLVGAPMQSTI
REEGRVFVYINSGSGAVMNAMETNLVGSDKYAARFGESIVNLGDIDNDGF
EDVAIGAPQEDDLQGAIYIYNGRADGISSTFSQRIEGLQISKSLSMFGQS
ISGQIDADNNGYVDVAVGAFRSDSAVLLRTRGSGSRKGGKRGSKYGPPCP
PCPAPEFLGGPSVFLFPPKPKDQLMISRTPEVTCVVVDVSQEDPEVQFNW
YVDGVEVHNAKTKPREEQFNSTYRVVSVLTVLHQDWLSGKEYKCKVSSKG
LPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSV
LHEALHNHYTQKSLSLSLGKDEGGEDGSGSSPGQGTQSENSCTHFPGNLP
NMLRDLRDAFSRVKTFFQMKDQLDNLLLKESLLEDFKGYLGCQALSEMIQ
FYLEEVMPQAENQDPDIKAHVNSLGENLKTLRLRLRRCHRFLPCENKSKA
VEQVKNAFNKLQEKGIYKAMSEFDIFINYIEAYMTMKIRN.

In embodiments, a chimeric protein comprises a variant of a ITGA4-Alpha-IL10 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 96.

### ITGB7-Beta-IL10

In embodiments, the chimeric protein is capable of contemporaneously binding the ITGB7 ligand and the IL10 receptor. In embodiments, the ITGB7 ligand, in combination with ITGA4, is MADCAM, and the IL10 receptor is IL10R. Integrin beta 7 subunit (ITGB7), in combination with ITGA4, binds with MADCAM. Interleukin-10 binds with its receptor Interleukin-10 receptor (IL10R) and functions as a key anti-inflammatory cytokine that can inhibit proinflammatory responses of both innate and adaptive immune cells, particularly in the intestinal mucosa. Accordingly, without wishing to be bound by theory, a chimeric protein comprising the extracellular domains of ITGB7 and IL10 is capable of contemporaneously preventing the entry of potentially pathogentic lymphocytes to the intestinal mucosa (via ITGB7) and activating an immune inhibitory signal (via IL10). In embodiments, this chimeric protein is referred to herein as ITGB7-Beta-IL10.

In embodiments, the chimeric proteins of the present disclosure comprise variants of the extracellular domain, which includes the ligand-binding domain, of ITGB7. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of ITGB7, e.g., human ITGB7.

In embodiments the extracellular domain of ITGB7 has the following amino acid sequence:

(SEQ ID NO: 90)
MVALPMVLVLLLVLSRGESELDAKIPSTGDATEWRNPHLSMLGSCQPAPS
CQKCILSHPSCAWCKQLNFTASGEAEARRCARREELLARGCPLEELEEPR
GQQEVLQDQPLSQGARGEGATQLAPQRVRVTLRPGEPQQLQVRFLRAEGY
PVDLYYLMDLSYSMKDALERVRQLGHALLVRLQEVTHSVRIGFGSFVDKT
VLPFVSTVPSKLRHPCPTRLERCQSPFSFHHVLSLTGDAQAFEREVGRQS
VSGNLDSPEGGFDAILQAALCQEQIGWRNVSRLLVFTSDDTFHTAGDGKL
GGIFMPSDGHCHLDSNGLYSRSTEFDYPSVGQVAQALSAANIQPIFAVTS
AALPVYQELSKLIPKSAVGELSEDSSNVVQLIMDAYNSLSSTVTLEHSSL
PPGVHISYESQCEGPEKREGKAEDRGQCNHVRINQTVTFWVSLQATHCLP
EPHLLRLRALGFSEELIVELHTLC.

In embodiments, a chimeric protein comprises a variant of the extracellular domain of ITGB7. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 90.

In embodiments, the first domain of a chimeric protein comprises an amino acid sequence that is at least 95% identical to the amino acid sequence of SEQ ID NO: 90.

One of ordinary skill may select variants of the known amino acid sequence of ITGB7 by consulting the literature, e.g., Sun et al., “Transmission of integrin beta7 transmembrane domain topology enables gut lymphoid tissue development,” J. Cell Biol. 217 (4), 1453-1465 (2018); Erle et al., “Lung epithelial lining fluid T cell subsets defined by distinct patterns of beta 7 and beta 1 integrin expression,” Am. J. Respir. Cell Mol. Biol. 10 (3), 237-244 (1994); Jiang et al, “The gene organization of the human beta 7 subunit, the common beta subunit of the leukocyte integrins HML-1 and LPAM-1,” Int. Immunol. 4 (9), 1031-1040 (1992); Erle et al, “Complete amino acid sequence of an integrin beta subunit (beta 7) identified in leukocytes,” J. Biol. Chem. 266 (17), 11009-11016 (1991), each of which is incorporated by reference in its entirety.

In embodiments, the chimeric proteins of the present disclosure comprise the extracellular domain of IL10 which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of IL10, e.g., human IL10.

In embodiments, the extracellular domain of I10 has the following amino acid sequence:

(SEQ ID NO: 95)
SPGQGTQSENSCTHFPGNLPNMLRDLRDAFSRVKTFFQMKDQLDNLLLKE
SLLEDFKGYLGCQALSEMIQFYLEEVMPQAENQDPDIKAHVNSLGENLKT
LRLRLRRCHRFLPCENKSKAVEQVKNAFNKLQEKGIYKAMSEFDIFINYI
EAYMTMKIRN.

In embodiments, a chimeric protein comprises a variant of the extracellular domain of IL10. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 95.

In embodiments, the second domain of a chimeric protein comprises an amino acid sequence that is at least 95% identical to the amino acid sequence of SEQ ID NO: 95.

One of ordinary skill may select variants of the known amino acid sequence of IL10 by consulting the literature, e.g., Cook et al., “Crystallization and preliminary X-ray investigation of recombinant human interleukin 10,” Proteins 22 (2), 187-190 (1995); Walter et al., “Crystal structure of interleukin 10 reveals an interferon gamma-like fold,” Biochemistry 34 (38), 12118-12125 (1995), each of which is incorporated by reference in its entirety.

In embodiments, a chimeric protein of the present disclosure comprises: (1) a first domain comprising the amino acid sequence of SEQ ID NO: 90, (b) a second domain comprises the amino acid sequence of SEQ ID NO: 95, and (c) a linker comprises an amino acid sequence that is at least 95% identical to SEQ ID NO: 1, SEQ ID NO: 2, SEQ ID NO: 3, or the linker underlined and/or in bold in SEQ ID NO: 97 below.

In embodiments, a ITGB7-Beta-IL10 chimeric protein of the present disclosure has the following amino acid sequence:

(SEQ ID NO: 97)
MVALPMVLVLLLVLSRGESELDAKIPSTGDATEWRNPHLSMLGSCQPAPS
CQKCILSHPSCAWCKQLNFTASGEAEARRCARREELLARGCPLEELEEPR
GQQEVLQDQPLSQGARGEGATQLAPQRVRVTLRPGEPQQLQVRFLRAEGY
PVDLYYLMDLSYSMKDALERVRQLGHALLVRLQEVTHSVRIGFGSFVDKT
VLPFVSTVPSKLRHPCPTRLERCQSPFSFHHVLSLTGDAQAFEREVGRQS
VSGNLDSPEGGFDAILQAALCQEQIGWRNVSRLLVFTSDDTFHTAGDGKL
GGIFMPSDGHCHLDSNGLYSRSTEFDYPSVGQVAQALSAANIQPIFAVTS
AALPVYQELSKLIPKSAVGELSEDSSNVVQLIMDAYNSLSSTVTLEHSSL
PPGVHISYESQCEGPEKREGKAEDRGQCNHVRINQTVTFWVSLQATHCLP
EPHLLRLRALGFSEELIVELHTLCGSGSDEGGEDGSKYGPPCPPCPAPEF
LGGPSVFLFPPKPKDQLMISRTPEVTCVVVDVSQEDPEVQFNWYVDGVEV
HNAKTKPREEQFNSTYRVVSVLTVLHQDWLSGKEYKCKVSSKGLPSSIEK
TISKAKGQPREPQVYTLPPSQEEMTKNQVSLTCLVKGFYPSDIAVEWESN
GQPENNYKTTPPVLDSDGSFFLYSRLTVDKSRWQEGNVFSCSVLHEALHN
HYTQKSLSLSLGKRKGGKRGSGSSPGQGTQSENSCTHFPGNLPNMLRDLR
DAFSRVKTFFQMKDQLDNLLLKESLLEDFKGYLGCQALSEMIQFYLEEVM
PQAENQDPDIKAHVNSLGENLKTLRLRLRRCHRFLPCENKSKAVEQVKNA
FNKLQEKGIYKAMSEFDIFINYIEAYMTMKIRN.

In embodiments, a chimeric protein comprises a variant of a ITGB7-Beta-IL10 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 97.

### ITGA4-Alpha-IL12A

In embodiments, the chimeric protein is capable of contemporaneously binding the ITGA4 ligand and the IL12A ligand/receptor. In embodiments, the ITGA4 ligand, in combination with ITGB7, is MADCAM, and IL12A associates with IL27B. Integrin alpha 4 subunit (ITGA4), in combination with ITGB7, binds with MADCAM. Interleukin-12 subunit alpha (IL12A) associates with IL27B to form interleukin (IL)-35, a heterodimeric cytokine which functions to promote a non-canonical regulatory phenotype in T lymphocytes. Accordingly, without wishing to be bound by theory, a chimeric protein comprising the extracellular domain of ITGA4 and the extracellular domain of IL12A is capable of contemporaneously competitively inhibiting entry into a mucosal immune compartment (via ITGA4) and promoting an immune regulatory microenvironment (via IL12A). In embodiments, this chimeric protein is referred to herein as ITGA4-Alpha-IL12A.

In embodiments, the chimeric proteins of the present disclosure comprise variants of the extracellular domain, which includes the ligand-binding domain, of ITGA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of ITGA4, e.g., human ITGA4.

In embodiments, the extracellular domain of ITGA4 has the following amino acid sequence:

(SEQ ID NO: 88)
YNVDTESALLYQGPHNTLFGYSVVLHSHGANRWLLVGAPTANWLANASVI
NPGAIYRCRIGKNPGQTCEQLQLGSPNGEPCGKTCLEERDNQWLGVTLSR
QPGENGSIVTCGHRWKNIFYIKNENKLPTGGCYGVPPDLRTELSKRIAPC
YQDYVKKFGENFASCQAGISSFYTKDLIVMGAPGSSYWTGSLFVYNITTN
KYKAFLDKQNQVKFGSYLGYSVGAGHFRSQHTTEVVGGAPQHEQIGKAYI
FSIDEKELNILHEMKGKKLGSYFGASVCAVDLNADGFSDLLVGAPMQSTI
REEGRVFVYINSGSGAVMNAMETNLVGSDKYAARFGESIVNLGDIDNDGF
EDVAIGAPQEDDLQGAIYIYNGRADGISSTFSQRIEGLQISKSLSMFGQS
ISGQIDADNNGYVDVAVGAFRSDSAVLLRTR.

In embodiments, a chimeric protein comprises a variant of the extracellular domain of ITGA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 88.

In embodiments, the first domain of a chimeric protein comprises an amino acid sequence that is at least 95% identical to the amino acid sequence of SEQ ID NO: 88.

One of ordinary skill may select variants of the known amino acid sequence of ITGA4 by consulting the literature, e.g., Takada et al., “The primary structure of the alpha 4 subunit of VLA-4: homology to other integrins and a possible cell-cell adhesion function,” EMBO J. 8 (5), 1361-1368 (1989); Rosen et al., “Characterization of the alpha 4 integrin gene promoter,” Proc. Natl. Acad. Sci. U.S.A. 88 (10), 4094-4098 (1991); Szabo et al, “Identification of two variants of the human integrin alpha 4 subunit,” Mol. Immunol. 32 (17-18), 1453-1454 (1995), each of which is incorporated by reference in its entirety.

In embodiments, the chimeric proteins of the present disclosure comprise the extracellular domain of IL12A which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of IL12A, e.g., human IL12A.

In embodiments, the extracellular domain of IL12A has the following amino acid sequence:

(SEQ ID NO: 80)
RNLPVATPDPGMFPCLHHSQNLLRAVSNMLQKARQTLEFYPCTSEEIDHE
DITKDKTSTVEACLPLELTKNESCLNSRETSFITNGSCLASRKTSFMMAL
CLSSIYEDLKMYQVEFKTMNAKLLMDPKRQIFLDQNMLAVIDELMQALNF
NSETVPQKSSLEEPDFYKTKIKLCILLHAFRIRAVTIDRVMSYLNAS.

In embodiments, a chimeric protein comprises a variant of the extracellular domain of IL12A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 80.

In embodiments, the second domain of a chimeric protein comprises an amino acid sequence that is at least 95% identical to the amino acid sequence of SEQ ID NO: 80.

One of ordinary skill may select variants of the known amino acid sequence of IL12A by consulting the literature, e.g., Wu et al., “The Contribution of Interleukin-12 Genetic Variations to Taiwanese Lung Cancer Anticancer Res. 38 (11), 6321-6327 (2018); D'Andrea et al., “Production of natural killer cell stimulatory factor (interleukin 12) by peripheral blood mononuclear cells,” J. Exp. Med. 176 (5), 1387-1398 (1992); Sieburth et al., “Assignment of genes encoding a unique cytokine (IL12) composed of two unrelated subunits to chromosomes 3 and 5,” Genomics 14 (1), 59-62 (1992); Schoenhaut et al, “Cloning and expression of murine IL-12,” J. Immunol. 148 (11), 3433-3440 (1992), each of which is incorporated by reference in its entirety.

In embodiments, a chimeric protein of the present disclosure comprises: (1) a first domain comprising the amino acid sequence of SEQ ID NO: 88, (b) a second domain comprises the amino acid sequence of SEQ ID NO: 80, and (c) a linker comprises an amino acid sequence that is at least 95% identical to SEQ ID NO: 1, SEQ ID NO: 2, SEQ ID NO: 3, or the linker underlined and/or in bold in SEQ ID NO: 98 below.

In embodiments, a ITGA4-Alpha-IL12A chimeric protein of the present disclosure has the following amino acid sequence:

(SEQ ID NO: 98)
YNVDTESALLYQGPHNTLFGYSVVLHSHGANRWLLVGAPTANWLANASVI
NPGAIYRCRIGKNPGQTCEQLQLGSPNGEPCGKTCLEERDNQWLGVTLSR
QPGENGSIVTCGHRWKNIFYIKNENKLPTGGCYGVPPDLRTELSKRIAPC
YQDYVKKFGENFASCQAGISSFYTKDLIVMGAPGSSYWTGSLFVYNITTN
KYKAFLDKQNQVKFGSYLGYSVGAGHFRSQHTTEVVGGAPQHEQIGKAYI
FSIDEKELNILHEMKGKKLGSYFGASVCAVDLNADGFSDLLVGAPMQSTI
REEGRVFVYINSGSGAVMNAMETNLVGSDKYAARFGESIVNLGDIDNDGF
EDVAIGAPQEDDLQGAIYIYNGRADGISSTFSQRIEGLQISKSLSMFGQS
ISGQIDADNNGYVDVAVGAFRSDSAVLLRTRGSGSRKGGKRGSKYGPPCP
PCPAPEFLGGPSVFLFPPKPKDQLMISRTPEVTCVVVDVSQEDPEVQFNW
YVDGVEVHNAKTKPREEQFNSTYRVVSVLTVLHQDWLSGKEYKCKVSSKG
LPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSV
LHEALHNHYTQKSLSLSLGKDEGGEDGSGSRNLPVATPDPGMFPCLHHSQ
NLLRAVSNMLQKARQTLEFYPCTSEEIDHEDITKDKTSTVEACLPLELTK
NESCLNSRETSFITNGSCLASRKTSFMMALCLSSIYEDLKMYQVEFKTMN
AKLLMDPKRQIFLDQNMLAVIDELMQALNFNSETVPQKSSLEEPDFYKTK
IKLCILLHAFRIRAVTIDRVMSYLNAS.

In embodiments, a chimeric protein comprises a variant of a ITGA4-Alpha-IL12A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 98.

### ITGB7-Beta-IL27B

In embodiments, the chimeric protein is capable of contemporaneously binding the ITGB7 ligand and the IL27B ligand/receptor. In embodiments, the ITGB7 ligand, in combination with ITGA4, is MADCAM, and IL27B associates with IL12A to form IL35. Integrin beta 7 subunit (ITGB7) in combination with ITGA4, binds with MADCAM. Interleukin-27 subunit beta (IL27B), also known as Epstein-Barr virus induced gene 3 (EBI3), associates with IL12A to form the IL-35 interleukin, a heterodimeric cytokine which functions to promote a non-canonical regulatory phenotype in T lymphocytes. IL-35 exhibits anti-inflammatory properties, that can regulate T-helper cell development, suppress T-cell proliferation, and inhibit cytotoxic T-cell activity. Accordingly, without wishing to be bound by theory, a chimeric protein comprising the extracellular domains of ITGB7 and IL27B is capable of contemporaneously competitively inhibiting entry into a mucosal immune compartment (via ITGB7) and promoting an immune regulatory microenvironment (via IL27B). In embodiments, this chimeric protein is referred to herein as ITGB7-Beta-IL27B.

In embodiments, the chimeric proteins of the present disclosure comprise variants of the extracellular domain, which includes the ligand-binding domain, of ITGB7. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of ITGB7, e.g., human ITGB7.

In embodiments, the extracellular domain of ITGB7 has the following amino acid sequence:

(SEQ ID NO: 90)
MVALPMVLVLLLVLSRGESELDAKIPSTGDATEWRNPHLSMLGSCQPAPSCQKCILSHPSCAWCK
QLNFTASGEAEARRCARREELLARGCPLEELEEPRGQQEVLQDQPLSQGARGEGATQLAPQRVR
VTLRPGEPQQLQVRFLRAEGYPVDLYYLMDLSYSMKDALERVRQLGHALLVRLQEVTHSVRIGFG
SFVDKTVLPFVSTVPSKLRHPCPTRLERCQSPFSFHHVLSLTGDAQAFEREVGRQSVSGNLDSPE
GGFDAILQAALCQEQIGWRNVSRLLVFTSDDTFHTAGDGKLGGIFMPSDGHCHLDSNGLYSRSTE
FDYPSVGQVAQALSAANIQPIFAVTSAALPVYQELSKLIPKSAVGELSEDSSNVVQLIMDAYNSLSS
TVTLEHSSLPPGVHISYESQCEGPEKREGKAEDRGQCNHVRINQTVTFWVSLQATHCLPEPHLLR
LRALGFSEELIVELHTLC.

In embodiments, a chimeric protein comprises a variant of the extracellular domain of ITGB7. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 90.

In embodiments, the first domain of a chimeric protein comprises an amino acid sequence that is at least 95% identical to the amino acid sequence of SEQ ID NO: 90.

One of ordinary skill may select variants of the known amino acid sequence of ITGB7 by consulting the literature, e.g., Sun et al., “Transmission of integrin beta7 transmembrane domain topology enables gut lymphoid tissue development,” J. Cell Biol. 217 (4), 1453-1465 (2018); Erle et al., “Lung epithelial lining fluid T cell subsets defined by distinct patterns of beta 7 and beta 1 integrin expression,” Am. J. Respir. Cell Mol. Biol. 10 (3), 237-244 (1994); Jiang et al, “The gene organization of the human beta 7 subunit, the common beta subunit of the leukocyte integrins HML-1 and LPAM-1,” Int. Immunol. 4 (9), 1031-1040 (1992); Erle et al, “Complete amino acid sequence of an integrin beta subunit (beta 7) identified in leukocytes,” J. Biol. Chem. 266 (17), 11009-11016 (1991), each of which is incorporated by reference in its entirety.

In embodiments, the chimeric proteins of the present disclosure comprise the extracellular domain of IL27B which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of IL27B, e.g., human IL27B.

In embodiments, the extracellular domain of IL27B has the following amino acid sequence:

(SEQ ID NO: 82)
RKGPPAALTLPRVQCRASRYPIAVDCSWTLPPAPNSTSPVSFIATYRLGMAARGHSWPCLQQTPTS
TSCTITDVQLFSMAPYVLNVTAVHPWGSSSSFVPFITEHIIKPDPPEGVRLSPLAERQLQVQWEPPG
SWPFPEIFSLKYWIRYKRQGAARFHRVGPIEATSFILRAVRPRARYYVQVAAQDLTDYGELSDWSL
PATATMSLGK.

In embodiments, a chimeric protein comprises a variant of the extracellular domain of IL27B. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 82.

In embodiments, the second domain of a chimeric protein comprises an amino acid sequence that is at least 95% identical to the amino acid sequence of SEQ ID NO: 82.

One of ordinary skill may select variants of the known amino acid sequence of IL27B by consulting the literature, e.g., Iranshani et al., “Decreased Gene Expression of Epstein-Barr Virus-Induced Gene 3 (EBI-3) may Contribute to the Pathogenesis of Rheumatoid Arthritis,” Immunol. Invest. 48 (4), 367-377 (2019); Larousserie et al., “Expression of IL-27 in human Th1-associated granulomatous diseases,” J. Pathol. 202 (2), 164-171 (2004), each of which is incorporated by reference in its entirety.

In embodiments, a chimeric protein of the present disclosure comprises: (1) a first domain comprising the amino acid sequence of SEQ ID NO: 90, (b) a second domain comprises the amino acid sequence of SEQ ID NO: 82, and (c) a linker comprises an amino acid sequence that is at least 95% identical to SEQ ID NO: 1, SEQ ID NO: 2, SEQ ID NO: 3, or the linker underlined and/or in bold in SEQ ID NO: 99 below.

In embodiments, a ITGB7-Beta-IL27B chimeric protein of the present disclosure has the following amino acid sequence:

(SEQ ID NO: 99)
MVALPMVLVLLLVLSRGESELDAKIPSTGDATEWRNPHLSMLGSCQPAPSCQKCILSHPSCAWCK
QLNFTASGEAEARRCARREELLARGCPLEELEEPRGQQEVLQDQPLSQGARGEGATQLAPQRVR
VTLRPGEPQQLQVRFLRAEGYPVDLYYLMDLSYSMKDALERVRQLGHALLVRLQEVTHSVRIGFG
SFVDKTVLPFVSTVPSKLRHPCPTRLERCQSPFSFHHVLSLTGDAQAFEREVGRQSVSGNLDSPE
GGFDAILQAALCQEQIGWRNVSRLLVFTSDDTFHTAGDGKLGGIFMPSDGHCHLDSNGLYSRSTE
FDYPSVGQVAQALSAANIQPIFAVTSAALPVYQELSKLIPKSAVGELSEDSSNVVQLIMDAYNSLSS
TVTLEHSSLPPGVHISYESQCEGPEKREGKAEDRGQCNHVRINQTVTFWVSLQATHCLPEPHLLR
LRALGFSEELIVELHTLCGSGSDEGGEDGSKYGPPCPPCPAPEFLGGPSVFLFPPKPKDQLMISRT
PEVTCVVVDVSQEDPEVQFNWYVDGVEVHNAKTKPREEQFNSTYRVVSVLTVLHQDWLSGKEYK
CKVSSKGLPSSIEKTISKAKGQPREPQVYTLPPSQEEMTKNQVSLTCLVKGFYPSDIAVEWESNGQ
PENNYKTTPPVLDSDGSFFLYSRLTVDKSRWQEGNVFSCSVLHEALHNHYTQKSLSLSLGKRKGG
KRGSGSRKGPPAALTLPRVQCRASRYPIAVDCSWTLPPAPNSTSPVSFIATYRLGMAARGHSWP
CLQQTPTSTSCTITDVQLFSMAPYVLNVTAVHPWGSSSSFVPFITEHIIKPDPPEGVRLSPLAERQL
QVQWEPPGSWPFPEIFSLKYWIRYKRQGAARFHRVGPIEATSFILRAVRPRARYYVQVAAQDLTD
YGELSDWSLPATATMSLGK.

In embodiments, a chimeric protein comprises a variant of a ITGB7-Beta-IL27B chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 99.

### IL36R-Alpha-IL12A

In embodiments, the chimeric protein is capable of contemporaneously binding the IL36R ligand and the IL12A ligand/receptor. In embodiments, the IL36R ligand is interleukin (IL)-36, and IL12A associates with IL27B. Interleukin 36 receptor (IL36R), also known as interleukin-1 receptor-like 2 (IL1RL2), is a member of the IL1 cytokine receptor family. Binding of IL36R with its ligand induces pro-inflammatory effects on various target cells, such as keratinocytes, synoviocytes, dendritic cells and T cells. Interleukin-12 subunit alpha (IL12A) associates with IL27B to form interleukin (IL)-35, which is a heterodimeric cytokine which functions to promote a non-canonical regulatory phenotype in T lymphocytes. Accordingly, without wishing to be bound by theory, a chimeric protein comprising the extracellular domains of IL36R and IL12A is capable of contemporaneously competitively inhibiting activation of an immune signal (via IL36R) and promoting an immune regulatory microenvironment (via IL12A). In embodiments, this chimeric protein is referred to herein as IL36R-Alpha-IL12A.

In embodiments, the chimeric proteins of the present disclosure comprise variants of the extracellular domain, which includes the ligand/receptor-binding domain, of IL36R. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of IL36R, e.g., human IL36R.

In embodiments, the extracellular domain of IL36R has the following amino acid sequence:

(SEQ ID NO: 71)
DGCKDIFMKNEILSASQPFAFNCTFPPITSGEVSVTWYKNSSKIPVSKIIQSRIHQDETWILFLPMEW
GDSGVYQCVIKGRDSCHRIHVNLTVFEKHWCDTSIGGLPNLSDEYKQILHLGKDDSLTCHLHFPKS
CVLGPIKWYKDCNEIKGERFTVLETRLLVSNVSAEDRGNYACQAILTHSGKQYEVLNGITVSITERAG
YGGSVPKIIYPKNHSIEVQLGTTLIVDCNVTDTKDNTNLRCWRVNNTLVDDYYDESKRIREGVETHV
SFREHNLYTVNITFLEVKMEDYGLPFMCHAGVSTAYIILQLPAPDFRSKYGPP.

In embodiments, a chimeric protein comprises a variant of the extracellular domain of IL36R. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 71.

In embodiments, the first domain of a chimeric protein comprises an amino acid sequence that is at least 95% identical to the amino acid sequence of SEQ ID NO: 71.

One of ordinary skill may select variants of the known amino acid sequence of IL36R by consulting the literature, e.g., Tomuschat et al., “Altered expression of IL36gamma and IL36 receptor (IL1RL2) in the colon of patients with Hirschsprung's disease,” Pediatr. Surg. Int. 33 (2), 181-186 (2017); Penha et al., “IL-36 receptor is expressed by human blood and intestinal T lymphocytes and is dose-dependently activated via IL-36beta and induces CD4+ lymphocyte proliferation,” Cytokine 85, 18-25 (2016); Yi et al., “Structural and Functional Attributes of the Interleukin-36 Receptor,” J. Biol. Chem. 291 (32), 16597-16609 (2016), each of which is incorporated by reference in its entirety.

In embodiments, the chimeric proteins of the present disclosure comprise the extracellular domain of IL12A which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of IL12A, e.g., human IL12A.

In embodiments, the extracellular domain of IL12A has the following amino acid sequence:

(SEQ ID NO: 80)
RNLPVATPDPGMFPCLHHSQNLLRAVSNMLQKARQTLEFYPCTSEEIDHEDITKDKTSTVEACLPLE
LTKNESCLNSRETSFITNGSCLASRKTSFMMALCLSSIYEDLKMYQVEFKTMNAKLLMDPKRQIFLD
QNMLAVIDELMQALNFNSETVPQKSSLEEPDFYKTKIKLCILLHAFRIRAVTIDRVMSYLNAS.

In embodiments, a chimeric protein comprises a variant of the extracellular domain of IL12A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 80.

In embodiments, the second domain of a chimeric protein comprises an amino acid sequence that is at least 95% identical to the amino acid sequence of SEQ ID NO: 80.

One of ordinary skill may select variants of the known amino acid sequence of IL12A by consulting the literature, e.g., Wu et al., “The Contribution of Interleukin-12 Genetic Variations to Taiwanese Lung Cancer Anticancer Res. 38 (11), 6321-6327 (2018); D'Andrea et al., “Production of natural killer cell stimulatory factor (interleukin 12) by peripheral blood mononuclear cells,” J. Exp. Med. 176 (5), 1387-1398 (1992); Sieburth et al., “Assignment of genes encoding a unique cytokine (IL12) composed of two unrelated subunits to chromosomes 3 and 5,” Genomics 14 (1), 59-62 (1992); Schoenhaut et al, “Cloning and expression of murine IL-12,” J. Immunol. 148 (11), 3433-3440 (1992), each of which is incorporated by reference in its entirety.

In embodiments, a chimeric protein of the present disclosure comprises: (1) a first domain comprising the amino acid sequence of SEQ ID NO: 71, (b) a second domain comprises the amino acid sequence of SEQ ID NO: 80, and (c) a linker comprises an amino acid sequence that is at least 95% identical to SEQ ID NO: 1, SEQ ID NO: 2, SEQ ID NO: 3, or the linker underlined and/or in bold in SEQ ID NO: 100 below.

In embodiments, a IL36R-Alpha-IL12A chimeric protein of the present disclosure has the following amino acid sequence:

(SEQ ID NO: 100)
DGCKDIFMKNEILSASQPFAFNCTFPPITSGEVSVTWYKNSSKIPVSKIIQSRIHQDETWILFLPMEW
GDSGVYQCVIKGRDSCHRIHVNLTVFEKHWCDTSIGGLPNLSDEYKQILHLGKDDSLTCHLHFPKS
CVLGPIKWYKDCNEIKGERFTVLETRLLVSNVSAEDRGNYACQAILTHSGKQYEVLNGITVSITERA
GYGGSVPKIIYPKNHSIEVQLGTTLIVDCNVTDTKDNTNLRCWRVNNTLVDDYYDESKRIREGVET
HVSFREHNLYTVNITFLEVKMEDYGLPFMCHAGVSTAYIILQLPAPDFRGSGSRKGGKRGSKYGPP
CPPCPAPEFLGGPSVFLFPPKPKDQLMISRTPEVTCVVVDVSQEDPEVQFNWYVDGVEVHNAKT
KPREEQFNSTYRVVSVLTVLHQDWLSGKEYKCKVSSKGLPSSIEKTISKAKGQPREPQVYTLPPS
QEEMTKNQVSLTCLVKGFYPSDIAVEWESNGQPENNYKTTPPVLDSDGSFFLYSRLTVDKSRWQ
EGNVFSCSVLHEALHNHYTQKSLSLSLGKDEGGEDGSGSRNLPVATPDPGMFPCLHHSQNLLRA
VSNMLQKARQTLEFYPCTSEEIDHEDITKDKTSTVEACLPLELTKNESCLNSRETSFITNGSCLASR
KTSFMMALCLSSIYEDLKMYQVEFKTMNAKLLMDPKRQIFLDQNMLAVIDELMQALNFNSETVPQK
SSLEEPDFYKTKIKLCILLHAFRIRAVTIDRVMSYLNAS.

In embodiments, a chimeric protein comprises a variant of a IL36R-Alpha-IL12A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 100.

### IL36R-Beta-1L27B

In embodiments, the chimeric protein is capable of contemporaneously binding the IL36R ligand and the IL27B ligand/receptor. In embodiments, the IL36R ligand is interleukin (IL)-36, and IL27B associates with IL12A. Binding of IL36R with its ligand induces pro-inflammatory effects on various target cells, such as keratinocytes, synoviocytes, dendritic cells and T cells. Interleukin-27 subunit beta (IL27B), also known as Epstein-Barr virus induced gene 3 (EBI3), associates with IL12A to form the IL-35 interleukin a heterodimeric cytokine which functions to promote a non-canonical regulatory phenotype in T lymphocytes. IL-35 exhibits anti-inflammatory properties, that can regulate T-helper cell development, suppress T-cell proliferation, and inhibit cytotoxic T-cell activity. Accordingly, without wishing to be bound by theory, a chimeric protein comprising the extracellular domain of IL36R and the extracellular domain of IL27B is capable of contemporaneously competitively inhibiting an immune activating signal (via IL36R) and promoting an immune regulatory microenvironment (via IL27B). In embodiments, this chimeric protein is referred to herein as IL36R-Beta-IL27B.

In embodiments, the chimeric proteins of the present disclosure comprise variants of the extracellular domain, which includes the ligand/receptor-binding domain, of IL36R. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of IL36R, e.g., human IL36R.

In embodiments, the extracellular domain of IL36R has the following amino acid sequence:

(SEQ ID NO: 71)
DGCKDIFMKNEILSASQPFAFNCTFPPITSGEVSVTWYKNSSKIPVSKIIQSRIHQDETWILFLPMEW
GDSGVYQCVIKGRDSCHRIHVNLTVFEKHWCDTSIGGLPNLSDEYKQILHLGKDDSLTCHLHFPKS
CVLGPIKWYKDCNEIKGERFTVLETRLLVSNVSAEDRGNYACQAILTHSGKQYEVLNGITVSITERAG
YGGSVPKIIYPKNHSIEVQLGTTLIVDCNVTDTKDNTNLRCWRVNNTLVDDYYDESKRIREGVETHV
SFREHNLYTVNITFLEVKMEDYGLPFMCHAGVSTAYIILQLPAPDFRSKYGPP.

In embodiments, a chimeric protein comprises a variant of the extracellular domain of IL36R. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 71.

In embodiments, the first domain of a chimeric protein comprises an amino acid sequence that is at least 95% identical to the amino acid sequence of SEQ ID NO: 71.

One of ordinary skill may select variants of the known amino acid sequence of IL36R by consulting the literature, e.g., Tomuschat et al., “Altered expression of IL36gamma and IL36 receptor (IL1RL2) in the colon of patients with Hirschsprung's disease,” Pediatr. Surg. Int. 33 (2), 181-186 (2017); Penha et al., “IL-36 receptor is expressed by human blood and intestinal T lymphocytes and is dose-dependently activated via IL-36beta and induces CD4+ lymphocyte proliferation,” Cytokine 85, 18-25 (2016); Yi et al., “Structural and Functional Attributes of the Interleukin-36 Receptor,” J. Biol. Chem. 291 (32), 16597-16609 (2016), each of which is incorporated by reference in its entirety.

In embodiments, the chimeric proteins of the present disclosure comprise the extracellular domain of IL27B which includes the ligand/receptor binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the extracellular domain of IL27B, e.g., human IL27B.

In embodiments, the extracellular domain of IL27B has the following amino acid sequence:

(SEQ ID NO: 82)
RKGPPAALTLPRVQCRASRYPIAVDCSWTLPPAPNSTSPVSFIATYRLGMAARGHSWPCLQQTPTS
TSCTITDVQLFSMAPYVLNVTAVHPWGSSSSFVPFITEHIIKPDPPEGVRLSPLAERQLQVQWEPPG
SWPFPEIFSLKYWIRYKRQGAARFHRVGPIEATSFILRAVRPRARYYVQVAAQDLTDYGELSDWSL
PATATMSLGK.

In embodiments, a chimeric protein comprises a variant of the extracellular domain of IL27B. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 82.

In embodiments, the second domain of a chimeric protein comprises an amino acid sequence that is at least 95% identical to the amino acid sequence of SEQ ID NO: 82.

One of ordinary skill may select variants of the known amino acid sequence of IL27B by consulting the literature, e.g., Iranshani et al., “Decreased Gene Expression of Epstein-Barr Virus-Induced Gene 3 (EBI-3) may Contribute to the Pathogenesis of Rheumatoid Arthritis,” Immunol. Invest. 48 (4), 367-377 (2019); Larousserie et al., “Expression of IL-27 in human Th1-associated granulomatous diseases,” J. Pathol. 202 (2), 164-171 (2004), each of which is incorporated by reference in its entirety.

In embodiments, a chimeric protein of the present disclosure comprises: (1) a first domain comprising the amino acid sequence of SEQ ID NO: 71, (b) a second domain comprises the amino acid sequence of SEQ ID NO: 82, and (c) a linker comprises an amino acid sequence that is at least 95% identical to SEQ ID NO: 1, SEQ ID NO: 2, SEQ ID NO: 3, or the linker underlined and/or in bold in SEQ ID NO: 101 below.

In embodiments, a IL36R-Beta-IL27B chimeric protein of the present disclosure has the following amino acid sequence:

(SEQ ID NO: 101)
DGCKDIFMKNEILSASQPFAFNCTFPPITSGEVSVTWYKNSSKIPVSKIIQSRIHQDETWILFLPMEW
GDSGVYQCVIKGRDSCHRIHVNLTVFEKHWCDTSIGGLPNLSDEYKQILHLGKDDSLTCHLHFPKS
CVLGPIKWYKDCNEIKGERFTVLETRLLVSNVSAEDRGNYACQAILTHSGKQYEVLNGITVSITERA
GYGGSVPKIIYPKNHSIEVQLGTTLIVDCNVTDTKDNTNLRCWRVNNTLVDDYYDESKRIREGVET
HVSFREHNLYTVNITFLEVKMEDYGLPFMCHAGVSTAYIILQLPAPDFRGSGSDEGGEDGSKYGPP
CPPCPAPEFLGGPSVFLFPPKPKDQLMISRTPEVTCVVVDVSQEDPEVQFNWYVDGVEVHNAKT
KPREEQFNSTYRVVSVLTVLHQDWLSGKEYKCKVSSKGLPSSIEKTISKAKGQPREPQVYTLPPS
QEEMTKNQVSLTCLVKGFYPSDIAVEWESNGQPENNYKTTPPVLDSDGSFFLYSRLTVDKSRWQ
EGNVFSCSVLHEALHNHYTQKSLSLSLGKRKGGKRGSGSRKGPPAALTLPRVQCRASRYPIAVDC
SWTLPPAPNSTSPVSFIATYRLGMAARGHSWPCLQQTPTSTSCTITDVQLFSMAPYVLNVTAVHP
WGSSSSFVPFITEHIIKPDPPEGVRLSPLAERQLQVQWEPPGSWPFPEIFSLKYWIRYKRQGAARF
HRVGPIEATSFILRAVRPRARYYVQVAAQDLTDYGELSDWSLPATATMSLGK.

In embodiments, a chimeric protein comprises a variant of a IL36R-Beta-IL27B chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 101.

### TNFR2-Fc-TGF-beta

In embodiments, the chimeric protein is capable of contemporaneously binding the TNFR2 ligand and a ligand/receptor of a Type II transmembrane protein selected from BTNL2C-type lectin domain (CLEC) family members, GITRL, TL1A, IL-10, and TGF-beta. In embodiments, the CLEC family member is selected from AICL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+DC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D. In embodiments, this chimeric protein is referred to herein as TNFR2-Fc-Type II.

In embodiments, TNFR2 is the receptor that binds tumor necrosis factor-alpha (TNFa), which is a cytokine produced by lymphocytes and macrophages, that mediates the immune response by attracting additional white blood cells to sites of inflammation and through additional molecular mechanisms that initiate and amplify inflammation. TNFR2's binding to TNFa, helps decrease excess inflammation cause by, as examples, autoimmune diseases such as ankylosing spondylitis, juvenile rheumatoid arthritis, psoriasis, psoriatic arthritis, rheumatoid arthritis, and, potentially, in a variety of other disorders mediated by excess TNFa.

In embodiments, the chimeric proteins of the present disclosure comprise variants of the extracellular domain, which includes the ligand-binding domain, of TNFR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence with the known amino acid sequence of the extracellular domain of TNFR2, e.g., human TNFR2.

In embodiments, the extracellular domain of TNFR2 has the following amino acid sequence:

(SEQ ID NO: 102)
LPAQVAFTPYAPEPGSTCRLREYYDQTAQMCCSKCSPGQHAKVFCTKTSDTVCDSCEDSTYTQL
WNWVPECLSCGSRCSSDQVETQACTREQNRICTCRPGWYCALSKQEGCRLCAPLRKCRPGFGV
ARPGTETSDVCKPCAPGTFSNTTSSTDICRPHQICNVVAIPGNASMDAVCTSTSPTRSMAPGAVH
LPQPVSTRSQHTQPTPEPSTAPSTSFLLPMGPSPPAEGSTGD.

In embodiments, a chimeric protein comprises a variant of the extracellular domain of TNFR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 102.

In embodiments, the first domain of a chimeric protein comprises an amino acid sequence that is at least 95% identical to the amino acid sequence of SEQ ID NO: 102.

One of ordinary skill may select variants of the known amino acid sequence of TNFR2 by consulting the literature, e.g., Kohno et al., “A second tumor necrosis factor receptor gene product can shed a naturally occurring tumor necrosis factor inhibitor.” Proc. Natl. Acad. Sci. U.S.A. 87 (21), 8331-8335 (1990); Smith et al., “A receptor for tumor necrosis factor defines an unusual family of cellular and viral proteins.” Science 248 (4958), 1019-1023 (1990); Loetscher et al., “Purification and partial amino acid sequence analysis of two distinct tumor necrosis factor receptors from HL60 cells.” J. Biol. Chem. 265 (33), 20131-20138 (1990); Dembic, et al., “Two human TNF receptors have similar extracellular, but distinct intracellular, domain sequences.” Cytokine 2 (4), 231-237 (1990); Pennica et al., “Biochemical properties of the 75-kDa tumor necrosis factor receptor. Characterization of ligand binding, internalization, and receptor phosphorylation.” J. Biol. Chem. 267 (29), 21172-21178 (1992); and Park et al., “Structural basis for self-association and receptor recognition of human TRAF2.” Nature 398 (6727), 533-538 (1999), each of which is incorporated by reference in its entirety.

In embodiments, the chimeric proteins of the present disclosure comprise variants of the extracellular domain of a herein-described Type II transmembrane protein, e.g., selected from TGF-beta, 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL. In embodiments, the CLEC family member is selected from AICL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+DC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D. The amino acid sequence of the herein-described Type II transmembrane protein are publicly available, see, e.g., at the World Wide Web (www) uniprot.org and at the World Wide Web (www) ncbi.nlm.nih.gov/protein and in one or more of WO2018/157162; WO2018/157165; WO2018/157164; WO2018/157163; and WO2017/059168, the contents relevant to this embodiment are incorporated herein by reference in its entirety. Moreover, many of the herein-described Type II transmembrane proteins have been structurally characterized, e.g., by predictive algorithms and/or x-ray crystallography; again see (www) uniprot.org; the contents relevant to this embodiment are incorporated herein by reference in its entirety.

In embodiments, TGF-beta is the ligand that binds transforming growth factor β (TGF-β) receptor, which signal via heterotetrameric complexes of type I and type II dual specificity kinase receptors. Activation of TGF-β receptors induces signaling via formation of Smad complexes that are translocated to the nucleus where they act as transcription factors, as well as via non-Smad pathways, including the Erk1/2, JNK and p38 MAP kinase pathways, and the Src tyrosine kinase, phosphatidylinositol 3-kinase, and Rho GTPases.

In embodiments, the chimeric proteins of the present disclosure comprise variants of the extracellular domain, which includes the receptor-binding domain, of TGF-beta. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence with the known amino acid sequence of the extracellular domain of TGF-beta, e.g., human TGF-beta.

In embodiments, the extracellular domain of TGF-beta has the following amino acid sequence:

(SEQ ID NO: 103)
ALDTNYCFSSTEKNCCVRQLYIDFRKDLGWKWIHEPKGYHANFCLGPCP
YIWSLDTQYSKVLALYNQHNPGASAAPCCVPQALEPLPIVYYVGRKPKV
EQLSNMIVRSCKCS.

In embodiments, a chimeric protein comprises a variant of the extracellular domain of TGF-beta. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 103.

In embodiments, the first domain of a chimeric protein comprises an amino acid sequence that is at least 95% identical to the amino acid sequence of SEQ ID NO: 103.

Based on the published amino acid sequences and structural characterizations, a skilled artisan could readily determine sequence variants of the herein-described Type II transmembrane protein which retain (or enhance) the native ligand/receptor binding affinity or the Type II transmembrane protein. Examples of such variants may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of a herein-described Type II transmembrane protein, e.g., the human Type II transmembrane protein.

In embodiments, a chimeric protein of the present disclosure comprises: (1) a first domain comprising the amino acid sequence of SEQ ID NO: 102 or a variant thereof, as described above, (b) a second domain comprises the amino acid sequence of SEQ ID NO: 103, or a variant thereof, as described above, and (c) a linker comprises an amino acid sequence that is at least 95% identical to SEQ ID NO: 1, SEQ ID NO: 2, or SEQ ID NO: 3, or the linker underlined and/or in bold in SEQ ID NO: 104 below.

In embodiments, a TNFR2-fc-TGF-beta chimeric protein of the present disclosure has the following amino acid sequence:

(SEQ ID NO: 104)
LPAQVAFTPYAPEPGSTCRLREYYDQTAQMCCSKCSPGQHAKVFCTKTSDTVCDSCEDSTYTQL
WNWVPECLSCGSRCSSDQVETQACTREQNRICTCRPGWYCALSKQEGCRLCAPLRKCRPGFGV
ARPGTETSDVVCKPCAPGTFSNTTSSTDICRPHQICNVVAIPGNASMDAVCTSTSPTRSMAPGAV
HLPQPVSTRSQHTQPTPEPSTAPSTSFLLPMGPSPPAEGSTGDSKYGPPCPPCPAPEFLGGPSVF
LFPPKPKDQLMISRTPEVTCVVVDVSQEDPEVQFNWYVDGVEVHNAKTKPREEQFNSTYRVVSVL
TVLHQDWLSGKEYKCKVSSKGLPSSIEKTISNATGQPREPQVYTLPPSQEEMTKNQVSLTCLVKG
FYPSDIAVEWESNGQPENNYKTTPPVLDSDGSFFLYSRLTVDKSRWQEGNVFSCSVLHEALHNH
YTQKSLSLSLGKIEGRMDALDTNYCFSSTEKNCCVRQLYIDFRKDLGWKWIHEPKGYHANFCLGP
CPYIWSLDTQYSKVLALYNQHNPGASAAPCCVPQALEPLPIVYYVGRKPKVEQLSNMIVRSCKCS.

In embodiments, a chimeric protein comprises a variant of a TNFR2-fc-TGF-beta chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 104.

In any herein-disclosed aspect and embodiment, the chimeric protein may comprise an amino acid sequence having one or more amino acid mutations relative to any of the protein sequences disclosed herein. In embodiments, the one or more amino acid mutations may be independently selected from substitutions, insertions, deletions, and truncations.

In embodiments, the amino acid mutations are amino acid substitutions, and may include conservative and/or non-conservative substitutions. “Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, IIe; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt a-helices. As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g., selenocysteine, pyrrolysine, N-formylmethionine R-alanine, GABA and 6-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, R-alanine, fluoro-amino acids, designer amino acids such as § methyl amino acids, C α-methyl amino acids, N α-methyl amino acids, and amino acid analogs in general).

Mutations may also be made to the nucleotide sequences of the chimeric proteins by reference to the genetic code, including taking into account codon degeneracy.

In embodiments, a chimeric protein is capable of binding murine ligand(s)/receptor(s).

In embodiments, a chimeric protein is capable of binding human ligand(s)/receptor(s).

In embodiments, each extracellular domain (or variant thereof) of the chimeric protein binds to its cognate receptor or ligand with a KD of about 1 nM to about 5 nM, for example, about 1 nM, about 1.5 nM, about 2 nM, about 2.5 nM, about 3 nM, about 3.5 nM, about 4 nM, about 4.5 nM, or about 5 nM. In embodiments, the chimeric protein binds to a cognate receptor or ligand with a KD of about 5 nM to about 15 nM, for example, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 10.5 nM, about 11 nM, about 11.5 nM, about 12 nM, about 12.5 nM, about 13 nM, about 13.5 nM, about 14 nM, about 14.5 nM, or about 15 nM.

In embodiments, each extracellular domain (or variant thereof) of the chimeric protein binds to its cognate receptor or ligand with a KD of less than about 1 μM, about 900 nM, about 800 nM, about 700 nM, about 600 nM, about 500 nM, about 400 nM, about 300 nM, about 200 nM, about 150 nM, about 130 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human CSF1 with a KD of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry).

As used herein, a variant of an extracellular domain is capable of binding the receptor/ligand of a native extracellular domain. For example, a variant may include one or more mutations in an extracellular domain which do not affect its binding affinity to its receptor/ligand; alternately, the one or more mutations in an extracellular domain may improve binding affinity for the receptor/ligand; or the one or more mutations in an extracellular domain may reduce binding affinity for the receptor/ligand, yet not eliminate binding altogether.

In embodiments, the one or more mutations are located outside the binding pocket where the extracellular domain interacts with its receptor/ligand. In embodiments, the one or more mutations are located inside the binding pocket where the extracellular domain interacts with its receptor/ligand, as long as the mutations do not eliminate binding altogether. Based on the skilled artisan's knowledge and the knowledge in the art regarding receptor-ligand binding, s/he would know which mutations would permit binding and which would eliminate binding.

In embodiments, the chimeric protein exhibits enhanced stability, high-avidity binding characteristics, prolonged off-rate for target binding and protein half-life relative to single-domain fusion protein or antibody controls.

A chimeric protein of the present disclosure may comprise more than two extracellular domains. For example, the chimeric protein may comprise three, four, five, six, seven, eight, nine, ten, or more extracellular domains. A second extracellular domain may be separated from a third extracellular domain via a linker, as disclosed herein. Alternately, a second extracellular domain may be directly linked (e.g., via a peptide bond) to a third extracellular domain. In embodiments, a chimeric protein includes extracellular domains that are directly linked and extracellular domains that are indirectly linked via a linker, as disclosed herein.

### Linkers

In embodiments, the chimeric protein comprises a linker.

In embodiments, the linker comprising at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

In a chimeric protein of the present disclosure, the linker is a polypeptide selected from a flexible amino acid sequence, an IgG hinge region, or an antibody sequence.

In embodiments, the linker is derived from naturally-occurring multi-domain proteins or is an empirical linker as described, for example, in Chichili et a., (2013), Protein Sci. 22(2):153-167, Chen et a., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. a!., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, IgG4, IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH2 domain and includes residues in C_{H2}. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present disclosure comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)).

In a chimeric protein of the present disclosure, the linker comprises a hinge-CH2-CH3 Fc domain derived from IgG4. In embodiments, the linker comprises a hinge-CH2-CH3 Fc domain derived from a human IgG4. In embodiments, the linker comprises an amino acid sequence that is at least 95% identical to the amino acid sequence of any one of SEQ ID NO: 1 to SEQ ID NO: 3, e.g., at least 95% identical to the amino acid sequence of SEQ ID NO: 2. In embodiments, the linker comprises one or more joining linkers, such joining linkers independently selected from SEQ ID NO: 4 to SEQ ID NO: 50 (or a variant thereof). In embodiments, the linker comprises two or more joining linkers each joining linker independently selected from SEQ ID NO: 4 to SEQ ID NO: 50 (or a variant thereof); wherein one joining linker is N terminal to the hinge-CH2-CH3 Fc domain and another joining linker is C terminal to the hinge-CH2-CH3 Fc domain.

In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, 1253A, H310A, M428L, H433K, N434A, N434F, N434S, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AAA mutation. In embodiments, the IgG constant region comprises an 1253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et a!. Journal of Immunology. (2015), 194(11):5497-508, and U.S. Pat. No. 7,083,784, the entire contents of which are hereby incorporated by reference.

An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (e.g. other than FcRn) with effector function.

In embodiments, the Fc domain in a linker has the amino acid sequence of SEQ ID NO: 1 (see Table 1, below), or at least 90%, or 93%, or 95%, or 97%, or 98%, or 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 1, below), or at least 90%, or 93%, or 95%, or 97%, or 98%, or 99% identity thereto. For instance, in embodiments, the Fc domain in a linker comprises the amino acid sequence of SEQ ID NO: 3 (see Table 1, below), or at least 90%, or 93%, or 95%, or 97%, or 98%, or 99% identity thereto.

Further, one or more joining linkers may be employed to connect an Fc domain in a linker (e.g., one of SEQ ID NO: 1, SEQ ID NO: 2, SEQ ID NO: 3 or at least 90%, or 93%, or 95%, or 97%, or 98%, or 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NO: 4 to SEQ ID NO: 50, or variants thereof are located between an extracellular domain as disclosed herein and an Fc domain as disclosed herein.

In embodiments, the present chimeric proteins may comprise variants of the joining linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 4 to SEQ ID NO: 50.

In embodiments, the first and second joining linkers may be different or they may be the same.

Without wishing to be bound by theory, including a linker comprising at least a part of an Fc domain in a chimeric protein, helps avoid formation of insoluble and, likely, non-functional protein concatenated oligomers and/or aggregates. This is in part due to the presence of cysteines in the Fc domain which are capable of forming disulfide bonds between chimeric proteins.

In embodiments, a chimeric protein may comprise one or more joining linkers, as disclosed herein, and lack a Fc domain linker, as disclosed herein.

In embodiments, the first and/or second joining linkers are independently selected from the amino acid sequences of SEQ ID NO: 4 to SEQ ID NO: 50 and are provided in Table 1 below:

Table 1
Illustrative linkers (Fc domain linkers and joining linkers)

| SEQ ID | |
|---|---|
| NO. | Sequence |
| 1 | APEFLGGPSVFLFPPKPKDTLMISRTPEVTCVVVDVSQEDPEVQFNWYVDGVEVHNAKTKPR |
| | EEQFNSTYRVVSVLTVLHQDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSSW |
| | QEGNVFSCSVMHEALHNHYTQKSLSLSLGK |
| 2 | APEFLGGPSVFLFPPKPKDQLMISRTPEVTCVVVDVSQEDPEVQFNWYVDGVEVHNAKTKPR |
| | EEQFNSTYRVVSVLTTPHSDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSSW |
| | QEGNVFSCSVLHEALHNHYTQKSLSLSLGK |
| 3 | APEFLGGPSVFLFPPKPKDQLMISRTPEVTCVVVDVSQEDPEVQFNWYVDGVEVHNAKTKPR |
| | EEQFNSTYRVVSVLTVLHQDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSRW |
| | QEGNVFSCSVLHEALHNHYTQKSLSLSLGK |
| 4 | SKYGPPCPSCP |
| 5 | SKYGPPCPPCP |
| 6 | SKYGPP |
| 7 | IEGRMD |
| 8 | GGGVPRDCG |
| 9 | IEGRMDGGGGAGGGG |
| 10 | GGGSGGGS |
| 11 | GGGSGGGGSGGG |
| 12 | EGKSSGSGSESKST |
| 13 | GGSG |
| 14 | GGSGGGSGGGSG |
| 15 | EAAAKEAAAKEAAAK |
| 16 | EAAAREAAAREAAAREAAAR |
| 17 | GGGGSGGGGSGGGGSAS |
| 18 | GGGGAGGGG |
| 19 | GS or GGS or LE |
| 20 | GSGSGS |
| 21 | GSGSGSGSGS |
| 22 | GGGGSAS |
| 23 | APAPAPAPAPAPAPAPAPAP |
| 24 | CPPC |
| 25 | GGGGS |
| 26 | GGGGGGGGS |
| 27 | GGGGSGGGGSGGGGS |
| 28 | GGGGSGGGGSGGGGSGGGGS |
| 29 | GGGGSGGGGSGGGGSGGGGSGGGGS |
| 30 | GGGGSGGGGSGGGGSGGGGSGGGGSGGGGS |
| 31 | GGGGSGGGGSGGGGSGGGGSGGGGSGGGGSGGGGS |
| 32 | GGGGSGGGGSGGGGSGGGGSGGGGSGGGGSGGGGSGGGGS |
| 33 | GGSGGSGGGGSGGGGS |
| 34 | GGGGGGGG |
| 35 | GGGGGG |
| 36 | EAAAK |
| 37 | EAAAKEAAAK |
| 38 | EAAAKEAAAKEAAAK |
| 39 | AEAAAKEAAAKA |
| 40 | AEAAAKEAAAKEAAAKA |
| 41 | AEAAAKEAAAKEAAAKEAAAKA |
| 42 | AEAAAKEAAAKEAAAKEAAAKEAAAKA |
| 43 | AEAAAKEAAAKEAAAKEAAAKALEAEAAAKEAAAKEAAAKEAAAKA |
| 44 | PAPAP |
| 45 | KESGSVSSEQLAQFRSLD |
| 46 | GSAGSAAGSGEF |
| 47 | GGGSE |
| 48 | GSESG |
| 49 | GSEGS |
| 50 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly₄Ser)ₙ, where n is from about 1 to about 8, e.g., 1, 2, 3, 4, 5, 6, 7, or 8 (SEQ ID NO: 25 to SEQ ID NO: 32, respectively). In embodiments, the joining linker sequence is GGSGGSGGGGSGGGGS (SEQ ID NO: 33). Additional illustrative joining linkers include, but are not limited to, linkers having the sequence LE, (EAAAK)ₙ (n=1-3) (SEQ ID NO: 36 to SEQ ID NO: 38), A(EAAAK)ₙA (n=2-5) (SEQ ID NO: 39 to SEQ ID NO: 42), A(EAAAK)4ALEA(EAAAK)4A (SEQ ID NO: 43), PAPAP (SEQ ID NO: 44), KESGSVSSEQLAQFRSLD (SEQ ID NO: 45), GSAGSAAGSGEF (SEQ ID NO: 46), and (XP)ₙ, with X designating any amino acid, e.g., Ala, Lys, or Glu. In embodiments, thejoining linker is GGS. In embodiments, a joining linker has the sequence (Gly)ₙ where n is any number from 1 to 100, for example: (Gly)₈ (SEQ ID NO: 34) and (Gly)₆ (SEQ ID NO: 35).

In embodiments, the joining linker is one or more of GGGSE (SEQ ID NO: 47), GSESG (SEQ ID NO: 48), GSEGS (SEQ ID NO: 49), GEGGSGEGSSGEGSSSEGGGSEGGGSEGGGSEGGS (SEQ ID NO: 50), and ajoining linker of randomly placed G, 5, and E every 4 amino acid intervals.

In embodiments, where a chimeric protein comprises a first domain, one joining linker preceding an Fc domain, a second joining linker following the Fc domain, and a second domain, the chimeric protein may comprise the following structure:

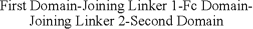

The combination of a first joining linker, an Fc Domain linker, and a second joining linker is referend to herein as a “modular linker”. In embodiments, a chimeric protein comprises a modular linker as shown in Table 2:

TABLE 2
Illustrative modular linkers

| | | | Modular Linker = Joining |
|---|---|---|---|
| Joining Linker | | Joining Linker | Linker 1 + Fc + |
| 1 | Fc | 2 | Joining Linker 2 |
| SKYGPPCPSC | APEFLGGPSVFLFPPKPKDTL | IEGRMD | SKYGPPCPSCPAPEFLGGPSV |
| P | MISRTPEVTCVVVDVSQEDPE | (SEQ ID NO: | FLFPPKPKDTLMISRTPEVTCV |
| (SEQ ID NO: 4) | VQFNWYVDGVEVHNAKTKPR | 7) | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 51) |
| SKYGPPCPSC | APEFLGGPSVFLFPPKPKDQL | IEGRMD | SKYGPPCPSCPAPEFLGGPSV |
| P | MISRTPEVTCVVVDVSQEDPE | (SEQ ID NO: | FLFPPKPKDQLMISRTPEVTCV |
| (SEQ ID NO: 4) | VQFNWYVDGVEVHNAKTKPR | 7) | WDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 52) |
| SKYGPPCPSC | APEFLGGPSVFLFPPKPKDQL | IEGRMD | SKYGPPCPSCPAPEFLGGPSV |
| P | MISRTPEVTCVVVDVSQEDPE | (SEQ ID NO: | FLFPPKPKDQLMISRTPEVTCV |
| (SEQ ID NO: 4) | VQFNWYVDGVEVHNAKTKPR | 7) | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 53) |
| SKYGPPCPPC | APEFLGGPSVFLFPPKPKDTL | IEGRMD | SKYGPPCPPCPAPEFLGGPSV |
| P | MISRTPEVTCVVVDVSQEDPE | (SEQ ID NO: | FLFPPKPKDTLMISRTPEVTCV |
| (SEQ ID NO: 5) | VQFNWYVDGVEVHNAKTKPR | 7 | VVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 54) |
| SKYGPPCPPC | APEFLGGPSVFLFPPKPKDQL | IEGRMD | SKYGPPCPPCPAPEFLGGPSV |
| P | MISRTPEVTCVVVDVSQEDPE | (SEQ ID NO: | FLFPPKPKDQLMISRTPEVTCV |
| (SEQ ID NO: 5) | VQFNWYVDGVEVHNAKTKPR | 7) | WDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 55) |
| SKYGPPCPPC | APEFLGGPSVFLFPPKPKDQL | IEGRMD | SKYGPPCPPCPAPEFLGGPSV |
| P | MISRTPEVTCVVVDVSQEDPE | (SEQ ID NO: | FLFPPKPKDQLMISRTPEVTCV |
| (SEQ ID NO: 5) | VQFNWYVDGVEVHNAKTKPR | 7) | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 56) |

In embodiments, the present chimeric proteins may comprise variants of the modular linkers disclosed in Table 2, above. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 51 to SEQ ID NO: 56.

In embodiments, the linker may be flexible, including without limitation highly flexible. In embodiments, the linker may be rigid, including without limitation a rigid alpha helix. Characteristics of illustrative joining linkers is shown below in Table 3:

TABLE 3
Characteristics of illustrative joining linkers

| Joining Linker Sequence | Characteristics |
|---|---|
| SKYGPPCPPCP (SEQ ID NO: 5) | IgG4 Hinge Region |
| IEGRMD (SEQ ID NO: 7) | Linker |
| GGGVPRDCG (SEQ ID NO: 8) | Flexible |
| GGGSGGGS (SEQ ID NO: 10) | Flexible |
| GGGSGGGGSGGG (SEQ ID NO: 11) | Flexible |
| EGKSSGSGSESKST (SEQ ID NO: 12) | Flexible + soluble |
| GGSG (SEQ ID NO: 13) | Flexible |
| GGSGGGSGGGSG (SEQ ID NO: 14) | Flexible |
| EAAAKEAAAKEAAAK (SEQ ID NO: 15) | Rigid Alpha Helix |
| EAAAREAAAREAAAREAAAR (SEQ ID NO: 16) | Rigid Alpha Helix |
| GGGGSGGGGSGGGGSAS (SEQ ID NO: 17) | Flexible |
| GGGGAGGGG (SEQ ID NO: 18) | Flexible |
| GS (SEQ ID NO: 19) | Highly flexible |
| GSGSGS (SEQ ID NO: 20) | Highly flexible |
| GSGSGSGSGS (SEQ ID NO: 21) | Highly flexible |
| GGGGSAS (SEQ ID NO: 22) | Flexible |
| APAPAPAPAPAPAPAPAPAP (SEQ ID NO: 23) | Rigid |

In embodiments, the linker may be functional. For example, without limitation, the linker may function to improve the folding and/or stability, improve the expression, improve the pharmacokinetics, and/or improve the bioactivity of the present chimeric protein. In another example, the linker may function to target the chimeric protein to a particular cell type or location.

In embodiments, a chimeric protein comprises only one joining linkers.

In embodiments, a chimeric protein lacks joining linkers.

In embodiments, the linker is a synthetic linker such as polyethylene glycol (PEG).

In embodiments, a chimeric protein has a first domain which is sterically capable of binding its ligand/receptor and/or the second domain which is sterically capable of binding its ligand/receptor. Thus, there is enough overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is referred to as “slack”) may be normally present in the extracellular domain(s), normally present in the linker, and/or normally present in the chimeric protein (as a whole). Alternately, or additionally, an amino acid sequence (for example) may be added to one or more extracellular domains and/or to the linker to provide the slack needed to avoid steric hindrance. Any amino acid sequence that provides slack may be added. In embodiments, the added amino acid sequence comprises the sequence (Gly)ₙ where n is any number from 1 to 100. Additional examples of addable amino acid sequence include the joining linkers described in Table 1 and Table 3. In embodiments, a polyethylene glycol (PEG) linker may be added between an extracellular domain and a linker to provide the slack needed to avoid steric hindrance. Such PEG linkers are well known in the art.

### Nucleic Acids

In one aspect, the present disclosure provides an isolated polynucleotide encoding the chimeric protein of any of the embodiments disclosed herein.

In one aspect, the present disclosure provides a host cell comprising the vector of any of the embodiments disclosed herein. In one aspect, the present disclosure provides a host cell comprising an RNA (without limitations, e.g., mmRNA) encoring the chimeric protein of any of the embodiments disclosed herein. A host cell comprising the nucleic acid, e.g., the mmRNA of any of the embodiments disclosed herein.

In embodiments, the polynucleotide is RNA, optionally, an mRNA.

In embodiments, the polynucleotide is or comprises an mRNA or a modified mRNA (mmRNA). The modified polypeptide may include a polynucleotide modification including, but not limited to, a nucleoside modification. In embodiments, the polynucleotide is or comprises an mmRNA. In embodiments, the mmRNA comprises one or more nucleoside modifications. In embodiments, the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

In embodiments, the mmRNA does not cause a substantial induction of the innate immune response of a cell into which the mmRNA is introduced. In embodiments, the modification in the mmRNA enhance one or more of the efficiency of production of the chimeric protein, intracellular retention of the mmRNA, and viability of contacted cells, and possess reduced immunogenicity.

In embodiments, the mmRNA has a length sufficient to include an open reading frame encoding the chimeric protein of the present disclosure.

Modified mRNAs need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least about 50% modified nucleotides, at least about 80% modified nucleotides, or at least about 90% modified nucleotides.

In embodiments, the mmRNAs may contain a modified pyrimidine such as uracil or cytosine. In embodiments, at least about 5%, at least about 10%, at least about 25%, at least about 50%, In embodiments, the modified uracil may be replaced by a compound having a single unique structure, or can be replaced by a plurality of compounds having different structures disclosed above (e.g., same mmRNA may contain 2, 3, 4 or more types of uniquely modified uracil). In embodiments, at least about 5%, at least about 10%, at least about 25%, at least about 50%, at least about 80%, at least about 90% or 100% of the cytosine in the nucleic acid may be replaced with a modified cytosine. The modified cytosine can be replaced by a compound having a single unique structure, or can be replaced by a plurality of compounds having different structures disclosed above (e.g., same mmRNA may contain 2, 3, 4 or more types of uniquely modified cytosine).

In embodiments, modified nucleosides include pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In embodiments, the nucleotide can be modified on the major groove face and can include replacing hydrogen on C-5 of uracil with a methyl group or a halo group.

In embodiments, a modified nucleoside is 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine or 5′-O-(1-Thiophosphate)-Pseudouridine.

Further examples of modified nucleotides and modified nucleotide combinations are disclosed in U.S. Pat. Nos. 8,710,200; 8,822,663; 8,999,380; 9,181,319; 9,254,311; 9,334,328; 9,464,124; 9,950,068; 10,626,400; 10,808,242; 11,020,477, and WO 2014/028429, the entire contents of which are hereby incorporated by reference. The methods for synthesizing the modified mRNA are disclosed, e.g., in US Patent Application Publication Nos. 20170204152, the entire contents of which are hereby incorporated by reference.

In embodiments, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the cytosine residues of the mmRNA are replaced by a modified cytosine residues. In embodiments, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the uracil residues of the mmRNA are replaced by a modified uracil residues.

In embodiments, the mmRNA further comprises a 5′ untranslated region (UTR) and/or a 3′UTR, wherein either or both may independently contain one or more different nucleoside modifications. In such embodiments, nucleoside modifications may also be present in the translatable region. In embodiments, the mmRNA further comprises a Kozak sequence. In embodiments, the mmRNA further comprises a internal ribosome entry site (IRES).

In embodiments, the mmRNA further comprises a 5′-cap and/or a poly A tail.

In embodiments, the 5′-cap contains a 5′-5′-triphosphate linkage between the 5′-most nucleotide and guanine nucleotide. In embodiments, the 5′-cap comprises a methylation of the ultimate and penultimate most 5′-nucleotides on the 2′-hydroxyl group. In embodiments, the 5′-cap facilitates binding the mRNA Cap Binding Protein (CBP), confers mRNA stability in the cell and/or confers translation competency.

In embodiments, the poly-A tail is greater than about 30 nucleotides, or greater than about 40 nucleotides in length. In embodiments, the poly-A tail is at least about 40 nucleotides, or at least about 45 nucleotides, or at least about 55 nucleotides, or at least about 60 nucleotides, or at least about 80 nucleotides, or at least about 90 nucleotides, or at least about 100 nucleotides, or at least about 120 nucleotides, or at least about 140 nucleotides, or at least about 160 nucleotides, or at least about 180 nucleotides, or at least about 200 nucleotides, or at least about 250 nucleotides, or at least about 300 nucleotides, or at least about 350 nucleotides, or at least about 400 nucleotides, or at least about 450 nucleotides, or at least about 500 nucleotides, or at least about 600 nucleotides, or at least about 700 nucleotides, or at least about 800 nucleotides, or at least about 900 nucleotides, or at least about 1000 nucleotides in length.

An aspect of the present disclosure relates to a nucleic acid encoding a chimeric protein of a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor. In embodiments, the polynucleotide is or comprises an mRNA or a modified mRNA (mmRNA) according to any of the embodiments disclosed herein.

In embodiments, the first domain comprises a transmembrane protein, a secreted protein, or a membrane-anchored extracellular protein selected from TNFR2, IL11RA, DR3, MADCAM, VCAM, IL36R, IL18BP, DcR3, OSMR, gp130, IL23R, IL12RB1, ITGA4, and ITGB7. In embodiments, the nucleic acid is RNA, optionally, an mRNA. In embodiments, the second domain comprises a transmembrane protein, a secreted protein, or a membrane-anchored extracellular protein selected from TGF-beta, DcR3, PD-L1, CCL20, CCL25, IL18BP, IL12A, IL27B, GITRL, and IL10.

In one aspect, the present disclosure relates to a nucleic acid encoding a chimeric protein, wherein the chimeric protein comprises a first domain comprising a portion of IL11RA and a second domain comprising a portion of DcR3. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of DR3 and a second domain comprising a portion of PD-L1. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of MADCAM and a second domain comprising a portion of CCL20. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of MADCAM and a second domain comprising a portion of CCL25. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of MADCAM and a second domain comprising a portion of PD-L1. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of VCAM and a second domain comprising a portion of PD-L1. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of IL36R and a second domain comprising a portion of DcR3. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of IL18BP and a second domain comprising a portion of DcR3. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of DcR3 and a second domain comprising a portion of IL18BP. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of OSMR and a second domain comprising a portion of DcR3. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of gp130 and a second domain comprising a portion of DcR3. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of DcR3 and a second domain comprising a portion of IL12A. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of DcR3 and a second domain comprising a portion of IL27B. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of IL23R and a second domain comprising a portion of DcR3. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of IL12RB1 and a second domain comprising a portion of DcR3. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of ITGA4 and a second domain comprising a portion of DcR3. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of ITGB7 and a second domain comprising a portion of DcR3. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of ITGA4 and a second domain comprising a portion of GITRL. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of ITGB7 and a second domain comprising a portion of GITRL. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of ITGA4 and a second domain comprising a portion of I10. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of ITGB7 and a second domain comprising a portion of I10. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of ITGA4 and a second domain comprising a portion of IL12A. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of ITGB7 and a second domain comprising a portion of IL27B. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of IL36R and a second domain comprising a portion of IL12A. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of IL36R and a second domain comprising a portion of IL27B. In embodiments, the nucleic acid is RNA, optionally, an mRNA. In embodiments, the polynucleotide is or comprises an mRNA or a modified mRNA (mmRNA) according to any of the embodiments disclosed herein.

In one aspect, the present disclosure relates to a nucleic acid encoding a chimeric protein, wherein the chimeric protein comprises a first domain comprising a portion of TNFR2 and a second domain comprising an extracellular domain of a transmembrane protein selected from TGF-beta, 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL; in embodiments, the second domain comprises TGF-beta. In embodiments, the CLEC family member is selected from AICL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+DC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D. In embodiments, the nucleic acid is RNA, optionally, an mRNA. In embodiments, the polynucleotide is or comprises an mRNA or a modified mRNA (mmRNA) according to any of the embodiments disclosed herein.

In another aspect, the present disclosure provides a nucleic acid encoding a chimeric protein, wherein the chimeric protein comprises: (a) a first domain comprising a portion of DR3 that is capable of binding a DR3 ligand/receptor (e.g. TL1A), (b) a second domain comprising a portion of PD-L1 that is capable of binding PD-1, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In yet another aspect, the present disclosure provides a nucleic acid encoding a chimeric protein, wherein the chimeric protein comprises: (a) a first domain comprising a portion of MADCAM that is capable of binding a MADCAM ligand/receptor, (b) a second domain comprising a portion of CCL20 that is capable of binding a CCL20 receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

An aspect of the present disclosure provides a nucleic acid encoding a chimeric protein, wherein the chimeric protein comprises: (a) a first domain comprising a portion of MADCAM that is capable of binding a MADCAM ligand/receptor, (b) a second domain comprising a portion of CCL25 that is capable of binding a CCL25 receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In another aspect, the present disclosure provides a nucleic acid encoding a chimeric protein, wherein the chimeric protein comprises: (a) a first domain comprising a portion of MADCAM that is capable of binding a MADCAM ligand/receptor, (b) a second domain comprising a portion of PD-L1 that is capable of binding PD-1, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In another aspect, the present disclosure provides a nucleic acid encoding a chimeric protein, wherein the chimeric protein comprises: (a) a first domain comprising a portion of VCAM that is capable of binding a VCAM ligand/receptor, (b) a second domain comprising a portion of PD-L1 that is capable of binding PD-1, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of IL36R that is capable of binding an IL36R ligand, (b) a second domain comprising a portion of DcR3 that is capable of binding a DcR3 ligand, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In another aspect, the present disclosure provides a nucleic acid encoding a chimeric protein, wherein the chimeric protein comprises: (a) a first domain comprising a portion of IL18BP that is capable of binding a IL18BP ligand/receptor, (b) a second domain comprising a portion of DcR3 that is capable of binding a DcR3 ligand, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In another aspect, the present disclosure provides a nucleic acid encoding a chimeric protein, wherein the chimeric protein comprises: (a) a first domain comprising a portion of DcR3 that is capable of binding a DcR3 ligand, (b) a second domain comprising a portion of IL18BP that is capable of binding a IL18BP ligand/receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In yet another aspect, the present disclosure provides a nucleic acid encoding a chimeric protein, wherein the chimeric protein comprises: (a) a first domain comprising a portion of OSMR that is capable of binding an OSMR ligand, (b) a second domain comprising a portion of DcR3 that is capable of binding a DcR3 ligand, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of gp130 that is capable of binding a gp130 ligand/receptor, (b) a second domain comprising a portion of DcR3 that is capable of binding a DcR3 ligand, and (c) a linker linking the first domain and the second domain and comprising a hinge-CH2-CH3 Fc domain.

In embodiments, the nucleic acid is RNA, optionally, an mRNA. In embodiments, the polynucleotide is or comprises an mRNA or a modified mRNA (mmRNA) according to any of the embodiments disclosed herein. In an aspect, the present disclosure provides a nucleic acid encoding a chimeric protein, wherein the chimeric protein comprises: (a) a first domain comprising a portion of DcR3 that is capable of binding a DcR3 ligand, (b) a second domain comprising a portion of IL12A that is capable of binding a IL12A ligand/receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In another aspect, the present disclosure provides a nucleic acid encoding a chimeric protein, wherein the chimeric protein comprises: (a) a first domain comprising a portion of DcR3 that is capable of binding a DcR3 ligand, (b) a second domain comprising a portion of IL27B that is capable of binding a IL27B ligand/receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of IL23R that is capable of binding an IL23R ligand/receptor, (b) a second domain comprising a portion of DcR3 that is capable of binding a DcR3 ligand, and (c) a linker linking the first domain and the second domain and comprising a hinge-CH2-CH3 Fc domain.

In embodiments, the nucleic acid is RNA, optionally, an mRNA. In embodiments, the polynucleotide is or comprises an mRNA or a modified mRNA (mmRNA) according to any of the embodiments disclosed herein. In yet another aspect, the present disclosure provides a nucleic acid encoding a chimeric protein, wherein the chimeric protein comprises: (a) a first domain comprising a portion of IL12RB1 that is capable of binding an IL12RB1 ligand/receptor, (b) a second domain comprising a portion of DcR3 that is capable of binding a DcR3 ligand, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of ITGA4 that is capable of binding an ITGA4 ligand/receptor, (b) a second domain comprising a portion of DcR3 that is capable of binding a DcR3 ligand, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of ITGB7 that is capable of binding an ITGB7 ligand/receptor, (b) a second domain comprising a portion of DcR3 that is capable of binding a DcR3 ligand, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of ITGA4 that is capable of binding an ITGA4 ligand/receptor, (b) a second domain comprising a portion of GITRL that is capable of binding a GITRL receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of ITGB7 that is capable of binding an ITGB7 ligand/receptor, (b) a second domain comprising a portion of GITRL that is capable of binding a GITRL ligand, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of ITGA4 that is capable of binding an ITGA4 ligand/receptor, (b) a second domain comprising a portion of IL10 that is capable of binding an I10 receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of ITGB7 that is capable of binding an ITGB7 ligand/receptor, (b) a second domain comprising a portion of IL10 that is capable of binding an I10 receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of ITGA4 that is capable of binding an ITGA4 ligand/receptor, (b) a second domain comprising a portion of IL12A that is capable of binding an IL12A ligand/receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of ITGB7 that is capable of binding an ITGB7 ligand/receptor, (b) a second domain comprising a portion of IL27B that is capable of binding an IL27B ligand/receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of IL36R that is capable of binding an IL36R ligand, (b) a second domain comprising a portion of IL12A that is capable of binding an IL12A ligand/receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In an aspect, the present disclosure provides a nucleic acid encoding a chimeric protein, wherein the chimeric protein comprises: (a) a first domain comprising a portion of IL36R that is capable of binding an IL36R ligand, (b) a second domain comprising a portion of IL27B that is capable of binding an IL27B ligand/receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

Another aspect of the present disclosure is a chimeric protein, or a nucleic acid encoding the chimeric protein, wherein the chimeric protein comprises: (a) a first domain comprising a portion of TNFR2 that is capable of binding a TNFR2 ligand/receptor, (b) a second domain comprising a portion of TGF-beta that is capable of binding a TGF-beta ligand/receptor, and (c) a linker linking the first domain and the second domain and comprising a hinge-CH2-CH3 Fc domain. In embodiments, the nucleic acid is RNA, optionally, an mRNA. In embodiments, the polynucleotide is or comprises an mRNA or a modified mRNA (mmRNA) according to any of the embodiments disclosed herein.

In embodiments, the polynucleotide is or comprises DNA. In embodiments, the polynucleotide is or comprises a minicircle or a plasmid DNA. In embodiments, the plasmid DNA is devoid of any prokaryotic components. In embodiments, the polynucleotide comprises a tissue-specific control element. In embodiments, the tissue-specific control element is a promoter or an enhancer. In embodiments, the plasmid DNA is an expression vector. In embodiments, the DNA is or comprises a minicircle. In embodiments, the minicircle is a circular molecule, which is optionally small. In embodiments, the minicircle utilizes a cellular transcription and translation machinery to produce an encoded gene product. In embodiments, the minicircle is devoid of any prokaryotic components. In embodiments, the minicircle only comprises substantially only sequences of mammalian origin (or those that have been optimized for mammalian cells). In embodiments, the minicircle lacks or has reduced amount of DNA sequence elements that are recognized by the innate immune system and/or toll-like receptors. In embodiments, the minicircle is produced by excising any bacterial components of from a parental plasmid, thereby making it smaller than a parental DNA sequence. In embodiments, the minicircle is of non-viral origin. In embodiments, the minicircle remains episomal. In embodiments, the minicircle does not replicate with a host cell. In embodiments, expression of the chimeric protein in non-dividing cells harboring a minicircle lasts for at least 2 days, or at least 4 days, or at least 6 days, or at least 8 days, or at least 10 days, or at least 12 days, or at least 14 days, or at least 16 days, or at least 18 days, or at least 20 days, or at least 22 days, or at least 24 days, or longer in dividing cells. In embodiments, expression of the chimeric protein in non-dividing cells harboring a minicircle lasts for at least 4 days, or at least 6 days, or at least 8 days, or at least 10 days, or at least 1 week, or at least 2 weeks, or at least 3 weeks, or at least 4 weeks, or at least 5 weeks, or at least 6 weeks, or at least 1 month, or at least 2 months, or at least 3 months, or at least 4 months, or at least 5 months, or at least 6 months, or at least 8 months, or longer in dividing cells.

In one aspect, the present disclosure provides a vector comprising the polynucleotide of any one of the embodiments disclosed herein. In embodiments, the chimeric protein can be provided as an expression vector. In embodiments, the expression vector is a DNA expression vector or an RNA expression vector. In embodiments, the expression vector is a viral expression vector. In embodiments, the expression vector is a non-viral expression vector (without limitation, e.g., a plasmid).

### Pharmaceutical Compositions

Aspects of the present disclosure include a pharmaceutical composition comprising the chimeric protein, or nucleic acid encoding the chimeric protein of any of the herein disclosed aspects or embodiments.

In embodiments, the chimeric protein in the pharmaceutical composition has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor.

In embodiments, the portion of the first domain is capable of binding the native ligand/receptor for the transmembrane protein, the secreted protein, or the membrane-anchored extracellular protein.

In embodiments, the portion of the second domain is capable of binding the native ligand/receptor for the transmembrane protein, the secreted protein, or the membrane-anchored extracellular protein.

In embodiments, the first domain comprises substantially the entire extracellular domain of the transmembrane protein, substantially the entire secreted protein, or substantially the entire membrane-anchored extracellular protein.

In embodiments, the second domain comprises substantially the entire extracellular domain of the transmembrane protein, substantially the entire secreted protein, or substantially the entire membrane-anchored extracellular protein.

In embodiments, the binding of the portion of the first domain to its ligand/receptor decreases immune system activity by activating an immune inhibitory signal or inhibiting an immune activating signal.

In embodiments, the binding of the portion of the second domain to its ligand/receptor decreases immune system activity by activating an immune inhibitory signal or by inhibiting an immune activating signal.

In embodiments, the portion of the first domain comprises a transmembrane protein, a secreted protein, or a membrane-anchored extracellular protein selected from TNFR2, IL11RA, DR3, MADCAM, VCAM, IL36R, IL18BP, DcR3, OSMR, gp130, IL23R, IL12RB1, ITGA4, and ITGB7.

In embodiments, the portion of the second domain comprises a transmembrane protein, a secreted protein, or a membrane-anchored extracellular protein selected from TGF-beta, DcR3, PD-L1, CCL20, CCL25, IL18BP, IL12A, IL27B, GITRL, and IL10.

In embodiments, the first domain comprises a portion of IL11RA and the second domain comprises a portion of DcR3.

In embodiments, the first domain comprises a portion of DR3 and the second domain comprises a portion of PD-L1.

In embodiments, the first domain comprises a portion of MADCAM and the second domain comprises a portion of CCL20.

In embodiments, the first domain comprises a portion of MADCAM and the second domain comprises a portion of CCL25.

In embodiments, the first domain comprises a portion of MADCAM and the second domain comprises a portion of PD-L1.

In embodiments, the first domain comprises a portion of VCAM and the second domain comprises a portion of PD-L1.

In embodiments, the first domain comprises a portion of IL36R and the second domain comprises a portion of DcR3.

In embodiments, the first domain comprises a portion of IL18BP and the second domain comprises a portion of DcR3.

In embodiments, the first domain comprises a portion of DcR3 and the second domain comprises a portion of IL18BP.

In embodiments, the first domain comprises a portion of OSMR and the second domain comprises a portion of DcR3.

In embodiments, the first domain comprises a portion of gp130 and the second domain comprises a portion of DcR3.

In embodiments, the first domain comprises a portion of DcR3 and the second domain comprises a portion of IL12A.

In embodiments, the first domain comprises a portion of DcR3 and the second domain comprises a portion of IL27B.

In embodiments, the first domain comprises a portion of IL23R and the second domain comprises a portion of DcR3.

In embodiments, the first domain comprises a portion of IL12RB1 and the second domain comprises a portion of DcR3.

In embodiments, the first domain comprises a portion of ITGA4 and the second domain comprises a portion of DcR3.

In embodiments, the first domain comprises a portion of ITGB7 and the second domain comprises a portion of DcR3.

In embodiments, the first domain comprises a portion of ITGA4 and the second domain comprises a portion of GITRL.

In embodiments, the first domain comprises a portion of ITGB7 and the second domain comprises a portion of GITRL.

In embodiments, the first domain comprises a portion of ITGA4 and the second domain comprises a portion of IL10.

In embodiments, the first domain comprises a portion of ITGB7 and the second domain comprises a portion of IL10.

In embodiments, the first domain comprises a portion of ITGA4 and the second domain comprises a portion of IL12A.

In embodiments, the first domain comprises a portion of ITGB7 and the second domain comprises a portion of IL27B.

In embodiments, the first domain comprises a portion of IL36R and the second domain comprises a portion of IL12A.

In embodiments, the first domain comprises a portion of IL36R and the second domain comprises a portion of IL27B.

In embodiments, the first domain comprises a portion of TNFR2 and the second domain comprises an extracellular domain of a transmembrane protein selected from TGF-beta, 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL; in embodiments, the second domain comprises an extracellular domain of TGF-beta. In embodiments, the CLEC family member is selected from AICL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+DC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D.

In embodiments, the binding of either or both of the first domain and the second domains to its ligand/receptor occurs with slow off rates (Koff), which provides a long interaction of a receptor and its ligand. In embodiments, the long interaction provides a prolonged decrease in immune system activity which comprises sustained activation of an immune inhibitory signal and/or a sustained inhibition of an immune activating signal. In embodiments, the sustained activation of the immune inhibitory signal and/or the sustained inhibition of the immune activating signal reduces the activity or proliferation of an immune cell, e.g., a B cell or a T cell. In embodiments, the sustained activation of the immune inhibitory signal and/or the sustained inhibition of the immune activating signal decreases synthesis and/or decreases release of a pro-inflammatory cytokine. In embodiments, the sustained activation of the immune inhibitory signal and/or the sustained inhibition of the immune activating signal increases synthesis and/or increases release of an anti-inflammatory cytokine. In embodiments, the sustained activation of the immune inhibitory signal and/or the sustained inhibition of the immune activating signal decreases antibody production and/or decreases secretion of antibodies by a B cell, e.g., an antibody that recognizes a self-antigen. In embodiments, the sustained activation of the immune inhibitory signal and/or the sustained inhibition of the immune activating signal decreases the activity of and/or decreases the number of T cytotoxic cells, e.g., which recognize a self-antigen and kill cells presenting or expressing the self-antigen. In embodiments, the sustained activation of the immune inhibitory signal and/or the sustained inhibition of the immune activating signal increases the activity and/or increases the number of T regulatory cells.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain, e.g., a hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of I111RA that is capable of binding a I111RA ligand, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL11RA-Fc-DcR3.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of DR3 that is capable of binding a DR3 ligand/receptor, (b) a second domain comprising a portion of PD-L1 that is capable of binding PD-1, and (c) a linker linking the first domain and the second domain and comprising a hinge-CH2-CH3 Fc domain. In some embodiments, this chimeric protein is referred to herein as DR3-Fc-PD-L1.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of MADCAM that is capable of binding a MADCAM ligand/receptor, (b) a second domain comprising a portion of CCL20 that is capable of binding a CCL20 receptor, and (c) a linker linking the first domain and the second domain and comprising a hinge-CH2-CH3 Fc domain. In some embodiments, this chimeric protein is referred to herein as MADCAM-Fc-CCL20.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of MADCAM that is capable of binding a MADCAM ligand/receptor, (b) a second domain comprising a portion of CCL25 that is capable of binding a CCL25 receptor, and (c) a linker linking the first domain and the second domain and comprising a hinge-CH2-CH3 Fc domain. In some embodiments, this chimeric protein is referred to herein as MADCAM-Fc-CCL25.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of MADCAM that is capable of binding a MADCAM ligand/receptor, (b) a second domain comprising a portion of PD-L1 that is capable of binding PD-1, and (c) a linker linking the first domain and the second domain and comprising a hinge-CH2-CH3 Fc domain. In some embodiments, this chimeric protein is referred to herein as MADCAM-Fc-PD-L1.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of VCAM that is capable of binding a VCAM ligand/receptor, (b) a second domain comprising a portion of PD-L1 that is capable of binding PD-1, and (c) a linker linking the first domain and the second domain and comprising a hinge-CH2-CH3 Fc domain. In some embodiments, this chimeric protein is referred to herein as VCAM-Fc-PD-L1.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of IL36R that is capable of binding an IL36R ligand, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL36R-Fc-DcR3.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of IL18BP that is capable of binding a IL18BP ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL18BP-Fc-DcR3.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of DcR3 that is capable of binding a DcR3 ligand, (b) a second domain comprising a portion of IL18BP that is capable of binding a IL18BP ligand/receptor, and (c) a linker linking the first domain and the second domain and comprising a hinge-CH2-CH3 Fc domain. In some embodiments, this chimeric protein is referred to herein as DcR3-Fc-IL18BP.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of OSMR that is capable of binding an OSMR ligand, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as OSMR-Alpha-DcR3.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of gp130 that is capable of binding a gp130 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as gp130-Beta-DcR3.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of DcR3 that is capable of binding a DcR3 ligand, (b) a second domain comprising a portion of IL12A that is capable of binding a IL12A ligand/receptor, and (c) a linker linking the first domain and the second domain and comprising a hinge-CH2-CH3 Fc domain. In some embodiments, this chimeric protein is referred to herein as DcR3-Alpha-IL12A.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of DcR3 that is capable of binding a DcR3 ligand, (b) a second domain comprising a portion of IL27B that is capable of binding a IL27B ligand/receptor, and (c) a linker linking the first domain and the second domain and comprising a hinge-CH2-CH3 Fc domain. In some embodiments, this chimeric protein is referred to herein as DcR3-Beta-IL27B.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of IL23R that is capable of binding an IL23R ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL23R-Alpha-DcR3.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of IL12RB1 that is capable of binding an IL12RB1 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL12RB1-Beta-DcR3.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of ITGA4 that is capable of binding an ITGA4 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as ITGA4-Alpha-DcR3.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of ITGB7 that is capable of binding an ITGB7 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as ITGB7-Beta-DcR3.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of ITGA4 that is capable of binding an ITGA4 ligand/receptor, (b) a second domain comprising a portion of GITRL that is capable of binding a GITRL ligand, and (c) a linker linking the first domain and the second domain and comprising a hinge-CH2-CH3 Fc domain. In some embodiments, this chimeric protein is referred to herein as ITGA4-Alpha-GITRL.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of ITGB7 that is capable of binding an ITGB7 ligand/receptor, (b) a second domain comprising a portion of GITRL that is capable of binding a GITRL ligand, and (c) a linker linking the first domain and the second domain and comprising a hinge-CH2-CH3 Fc domain. In some embodiments, this chimeric protein is referred to herein as ITGB7-Beta-GITRL.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of ITGA4 that is capable of binding an ITGA4 ligand/receptor, (b) a second domain comprising a portion of IL10 that is capable of binding an IL10 receptor, and (c) a linker linking the first domain and the second domain and comprising a hinge-CH2-CH3 Fc domain. In some embodiments, this chimeric protein is referred to herein as ITGA4-Alpha-IL10.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of ITGB7 that is capable of binding an ITGB7 ligand/receptor, (b) a second domain comprising a portion of IL10 that is capable of binding an IL10 receptor, and (c) a linker linking the first domain and the second domain and comprising a hinge-CH2-CH3 Fc domain. In some embodiments, this chimeric protein is referred to herein as ITGB7-Beta-IL10.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of ITGA4 that is capable of binding an ITGA4 ligand/receptor, (b) a second domain comprising a portion of IL12A that is capable of binding an IL12A ligand/receptor, and (c) a linker linking the first domain and the second domain and comprising a hinge-CH2-CH3 Fc domain. In some embodiments, this chimeric protein is referred to herein as ITGA4-Alpha-IL12A.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of ITGB7 that is capable of binding an ITGB7 ligand/receptor, (b) a second domain comprising a portion of IL27B that is capable of binding an IL27B ligand/receptor, and (c) a linker linking the first domain and the second domain and comprising a hinge-CH2-CH3 Fc domain. In some embodiments, this chimeric protein is referred to herein as ITGB7-Beta-IL27B.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of IL36R that is capable of binding an IL36R ligand, (b) a second domain comprising a portion of IL12A that is capable of binding an IL12A ligand/receptor, and (c) a linker linking the first domain and the second domain and comprising a hinge-CH2-CH3 Fc domain. In some embodiments, this chimeric protein is referred to herein as IL36R-Alpha-IL12A.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of IL36R that is capable of binding an IL36R ligand, (b) a second domain comprising a portion of IL27B that is capable of binding an IL27B ligand/receptor, and (c) a linker linking the first domain and the second domain and comprising a hinge-CH2-CH3 Fc domain. In some embodiments, this chimeric protein is referred to herein as IL36R-Beta-IL27B.

In embodiments, the pharmaceutical composition comprises a chimeric protein or a nucleic acid encoding the chimeric protein (without limitation, e.g., mmRNA), wherein the chimeric protein comprises: (a) a first domain comprising a portion of TNFR2 that is capable of binding a TNFR2 ligand/receptor, (b) a second domain comprising a portion of TGF-beta that is capable of binding a TGF-beta ligand/receptor, and (c) a linker linking the first domain and the second domain and comprising a hinge-CH2-CH3 Fc domain. In some embodiments, this chimeric protein is referred to as TNFR2-Fc-TGF-beta.

In embodiments, the hinge-CH2-CH3 Fc domain comprises at least one cysteine residue capable of forming a disulfide bond. In embodiments, the hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the chimeric protein in the pharmaceutical composition may be a recombinant fusion protein.

In embodiments, nucleic acid in the pharmaceutical composition may be a modified mRNA (mmRNA).

In one aspect, the present disclosure provides a pharmaceutical composition comprising a pharmaceutically acceptable excipient or carrier, and the chimeric protein of any of the embodiments disclosed herein, the isolated polynucleotide of any of the embodiments disclosed herein, the mmRNA of any of the embodiments disclosed herein, or the vector of any of the embodiments disclosed herein. In embodiments, the pharmaceutical composition comprises the mmRNA of any of the embodiments disclosed herein.

In embodiments, the carrier is a lipidoid, aliposome, a lipoplex, a lipid nanoparticle, a polymeric nanoparticle, a peptide, a protein, a cell, a nanoparticle mimic, a nanotube, or a conjugate. In embodiments, the pharmaceutical composition is formulated as a lipid nanoparticle (LNP), a lipoplex, or a liposome. In embodiments, the pharmaceutical composition is formulated as a lipid nanoparticle (LNP). In embodiments, the lipid nanoparticles comprise lipids selected from an ionizable lipid (e.g., an ionizable cationic lipid selected from DLin-DMA, DLin-K-DMA, DLin-KC2-DMA, DLin-MC3-DMA, 98N12-5, and C12-200); a structural lipid (e.g., distearoylphosphatidylcholine (DSPC)); cholesterol, and a polyethyleneglycol (PEG)-lipid (e.g., a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE).

In embodiments, the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In embodiments, the pharmaceutical composition is formulated for parenteral administration. In embodiments, the pharmaceutical composition is formulated for topical, dermal, intradermal, intramuscular, intraperitoneal, intraarticular, intravenous, subcutaneous, intraarterial or transdermal administration. In embodiments, the pharmaceutical composition is formulated for topical administration.

In one aspect, the present disclosure provides a pharmaceutical composition comprising a pharmaceutically acceptable excipient or carrier, and the chimeric protein of any one of the embodiments disclosed herein, the isolated polynucleotide of any one of the embodiments disclosed herein, the vector of the embodiments disclosed herein, or the host cell of any of the embodiments disclosed herein. In embodiments, the pharmaceutical composition comprises the nucleic acid, e.g., the mmRNA of any one of the embodiments disclosed herein.

In embodiments, the carrier is a lipidoid, aliposome, a lipoplex, a lipid nanoparticle, a polymeric nanoparticle, a peptide, a protein, a cell, a nanoparticle mimic, a nanotube, or a conjugate. In embodiments, the pharmaceutical composition is formulated as a lipid nanoparticles (LNPs), a lipoplex, or a liposome. In embodiments, the pharmaceutical composition is formulated as a lipid nanoparticles (LNPs).

In embodiments, the lipid nanoparticles comprise lipids selected from an ionizable lipid (e.g., an ionizable cationic lipid selected from DLin-DMA, DLin-K-DMA, DLin-KC2-DMA, DLin-MC3-DMA, 98N12-5, and C12-200); a structural lipid (e.g., distearoylphosphatidylcholine (DSPC)); cholesterol, and a polyethyleneglycol (PEG)-lipid (e.g., a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE); and the nucleic acid, e.g., the mmRNA.

In embodiments, the lipid nanoparticles comprise lipids selected from an ionizable lipid; a structural lipid; cholesterol, and a polyethyleneglycol (PEG)-lipid; 1,2-dioleoyl-3-trimethylammoniumpropane (DOTAP); dioleoylphosphatidylethanolamine (DOPE); and the nucleic acid, e.g., the mmRNA. In embodiments, the ionizable lipid is an ionizable cationic lipid selected from DLin-DMA, DLin-K-DMA, DLin-KC2-DMA, DLin-MC3-DMA, 98N12-5, and C12-200. In embodiments, the polyethyleneglycol (PEG)-lipid is selected from a PEG-diacylglycerol (DAG), a PEG-dialkyloxypropyl (DAA), a PEG-phospholipid, a PEG-ceramide (Cer), or a mixture thereof, or a PEG-dilauryloxypropyl (e.g., C12, a PEG-dimyristyloxypropyl (C14), a PEG-dipalmityloxypropyl (C16), or a PEG-distearyloxypropyl (C18)).

In embodiments, the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In embodiments, the isolated polynucleotide is or comprises a conjugated polynucleotide sequence that is introduced into cells by various transfection methods such as, e.g., methods that employ lipid particles. In embodiments, a composition, including a gene transfer construct, comprises a delivery particle. In embodiments, the delivery particle comprises a lipid-based particle (e.g., a lipid nanoparticle (LNP)), cationic lipid, or a biodegradable polymer). Lipid nanoparticle (LNP) delivery of gene transfer construct provides certain advantages, including transient, non-integrating expression to limit potential off-target events and immune responses, and efficient delivery with the capacity to transport large cargos. LNPs have been used for delivery of small interfering RNA (siRNA) and mRNA, and for in vitro and in vivo delivering CRISPR/Cas9 components to hepatocytes and the liver. For example, U.S. Pat. No. 10,195,291 describes the use of LNPs for delivery of RNA interference (RNAi) therapeutic agents.

In embodiments, the composition in accordance with embodiments of the present disclosure is in the form of a LNP. In embodiments, the LNP comprises one or more lipids selected from 1,2-dioleoyl-3-trimethylammonium propane (DOTAP); N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy(polyethylene glycol)-2000](DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC).

In embodiments, the composition can have a lipid and a polymer in various ratios, wherein the lipid can be selected from, e.g., DOTAP, DC-Chol, PC, Triolein, DSPE-PEG, and wherein the polymer can be, e.g., PEI or Poly Lactic-co-Glycolic Acid (PLGA). Any other lipid and polymer can be used additionally or alternatively.

In embodiments, the ratio of the lipid and the polymer is about 0.5:1, or about 1:1, or about 1:1.5, or about 1:2, or about 1:2.5, or about 1:3, or about 3:1, or about 2.5:1, or about 2:1, or about 1.5:1, or about 1:1, or about 1:0.5. In embodiments, the LNP comprises a cationic lipid, non-limiting examples of which include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-′)amino)ethyl)(2 hydroxydodecyl)amino)ethyl) piperazin-1-yl)ethylazanediyl)didodecan-2-ol (Tech G1), 1,2-Dilinoleyloxo-3-(2-N,N-dimethylamino) ethoxypropane (DLin-EG-DMA), or a mixture thereof.

In embodiments, the LNP comprises one or more molecules selected from polyethylenimine (PEI) and poly(lactic-co-glycolic acid) (PLGA), and N-Acetylgalactosamine (GaINAc), which are suitable for hepatic delivery. In embodiments, the LNP comprises a hepatic-directed compound as described, e.g., in U.S. Pat. No. 5,985,826, which is incorporated by reference herein in its entirety. GaINAc is known to target Asialoglycoprotein Receptor (ASGPR) expressed on mammalian hepatic cells. See Hu et al. Protein Pept Lett. 2014; 21(10):1025-30.

In some examples, the isolated polynucleotide can be formulated or complexed with PEI or a derivative thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In embodiments, the LNP is a conjugated lipid, non-limiting examples of which include a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (C12, a PEG-dimyristyloxypropyl (C14), a PEG-dipalmityloxypropyl (C16), or a PEG-distearyloxypropyl (C18).

In embodiments, a nanoparticle is a particle having a diameter of less than about 1000 nm. In embodiments, nanoparticles of the present disclosure have a greatest dimension (e.g., diameter) of about 500 nm or less, or about 400 nm or less, or about 300 nm or less, or about 200 nm or less, or about 100 nm or less. In embodiments, nanoparticles of the present disclosure have a greatest dimension ranging between about 50 nm and about 150 nm, or between about 70 nm and about 130 nm, or between about 80 nm and about 120 nm, or between about 90 nm and about 110 nm. In embodiments, the nanoparticles of the present disclosure have a greatest dimension (e.g., a diameter) of about 100 nm.

In embodiments, the isolated polynucleotide or mmRNA (and/or additional agents) are included various formulations. Any isolated polynucleotide or mmRNA (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In embodiments, the pharmaceutical composition further comprises an immunosuppressive agent. In embodiments, the immunosuppressive agent is selected from the group consisting of an antibody (e.g., basiliximab, daclizumab, and muromonab), an anti-immunophilin (e.g., cyclosporine, tacrolimus, and sirolimus), an antimetabolite (e.g., azathioprine and methotrexate), a cytostatic (such as alkylating agents), a cytotoxic antibiotic, an inteferon, a mycophenolate, an opioid, a small biological agent (e.g., fingolimod and myriocin), and a TNF binding protein.

In embodiments, the pharmaceutical composition further comprises an anti-inflammatory drug, e.g., a non-steroidal anti-inflammatory or a corticosteroid. In embodiments, the non-steroidal anti-inflammatory is selected from the group consisting of acetyl salicylic acid (aspirin), benzyl-2,5-diacetoxybenzoic acid, celecoxib, diclofenac, etodolac, etofenamate, fulindac, glycol salicylate, ibuprofen, indomethacin, ketoprofen, methyl salicylate, nabumetone, naproxen, oxaprozin, phenylbutazone, piroxicam, salicylic acid, salicylmides, and vimovo® (a combination of naproxen and esomeprazole magnesium). In embodiments, the corticosteroid is selected from the group consisting of alpha-methyl dexamethasone, amcinafel, amcinafide, beclomethasone dipropionate, beclomethasone dipropionate., betamethasone and the balance of its esters, betamethasone benzoate, betamethasone dipropionate, betamethasone valerate, beta-methyl betamethasone, bethamethasone, chloroprednisone, clescinolone, clobetasol valerate, clocortelone, cortisone, cortodoxone, desonide, desoxymethasone, dexamethasone, dichlorisone, diflorasone diacetate, diflucortolone valerate, difluorosone diacetate, difluprednate, fluadrenolone, flucetonide, fluclorolone acetonide, flucloronide, flucortine butylester, fludrocortisone, flumethasone pivalate, flunisolide, fluocinonide, fluocortolone, fluoromethalone, fluosinolone acetonide, fluperolone, fluprednidene (fluprednylidene) acetate, fluprednisolone, fluradrenolone acetonide, flurandrenolone, halcinonide, hydrocortisone, hydrocortisone acetate, hydrocortisone butyrate, hydroxyltriamcinolone, medrysone, meprednisone, methylprednisolone, paramethasone, prednisolone, prednisone, triamcinolone, and triamcinolone acetonide.

In embodiments, the pharmaceutical composition further comprises both an immunosuppressive agent and an anti-inflammatory drug.

In embodiments, the chimeric proteins (and/or an anti-inflammatory drug and/or an immunosuppressive agent) disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions disclosed herein are in the form of a pharmaceutically acceptable salt.

Further, any chimeric protein (and/or an anti-inflammatory drug and/or an immunosuppressive agent) disclosed herein can be administered to a subject as a component of pharmaceutical composition, that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent disclosed herein, if desired, can also comprise minor amounts of wetting or emulsifying agents, or pH buffering agents.

In embodiments, the chimeric proteins disclosed herein are resuspended in a saline buffer (including, without limitation TBS, PBS, and the like).

In embodiments, the chimeric proteins may by conjugated and/or fused with another agent to extend half-life or otherwise improve pharmacodynamic and pharmacokinetic properties. In embodiments, the chimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In embodiments, each of the individual chimeric proteins is fused to one or more of the agents described in BioDrugs (2015) 29:215-239, the entire contents of which are hereby incorporated by reference.

The present disclosure includes the disclosed chimeric protein (and/or an anti-inflammatory drug and/or an immunosuppressive agent) in various formulations of pharmaceutical composition. Any chimeric protein (and/or an anti-inflammatory drug and/or an immunosuppressive agent) disclosed herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the pharmaceutical compositions comprising the chimeric protein (and/or an anti-inflammatory drug and/or an immunosuppressive agent) can also include a solubilizing agent. Also, the agents can be delivered with a suitable vehicle or delivery device as known in the art. Combination therapies outlined herein can be co-delivered in a single delivery vehicle or delivery device. Pharmaceutical compositions for administration can optionally include a local anesthetic such as, for example, lignocaine to lessen pain at the site of the injection.

The pharmaceutical compositions comprising the chimeric protein (and/or an anti-inflammatory drug and/or an immunosuppressive agent) of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In embodiments, any chimeric protein (and/or an anti-inflammatory drug and/or an immunosuppressive agent) disclosed herein is formulated in accordance with routine procedures as a pharmaceutical composition adapted for a mode of administration disclosed herein.

### Methods of Treatment

An aspect of the present disclosure is a method of treating an autoimmune disease comprising administering to a subject in need thereof an effective amount of a pharmaceutical composition comprising the chimeric protein, or nucleic acid encoding the chimeric protein of any of the herein disclosed aspects or embodiments.

In some embodiments, the subject has an Inflammatory Bowel Disease (IBD) such as Crohn's disease or ulcerative colitis.

An aspect of the present disclosure is a method of treating Crohn's disease comprising administering to a subject in need thereof an effective amount of a pharmaceutical composition comprising the chimeric protein, or nucleic acid encoding the chimeric protein of any of the herein disclosed aspects or embodiments. Crohn's disease may affect any part of the gastrointestinal tract, including the small and large intestine. Complications may occur outside the gastrointestinal tract and may include anemia, skin rashes, arthritis, inflammation of the eye, and tiredness. While the cause of Crohn's disease is unknown, it is believed to be due to a combination of environmental, immune and bacterial factors in genetically susceptible individuals. It results in a chronic inflammatory disorder, in which the body's immune system attacks the gastrointestinal tract possibly directed at microbial antigens. Diagnosis is based on a number of findings including biopsy and appearance of the bowel wall, medical imaging and description of the disease. There are no medications or surgical procedures that can cure Crohn's disease. Treatment options help with symptoms, maintain remission, and prevent relapse, and include corticosteroid and methotrexate, which can be used in conjunction with the present disclosure.

An aspect of the present disclosure is a method of treating ulcerative colitis comprising administering to a subject in need thereof an effective amount of a pharmaceutical composition comprising the chimeric protein, or nucleic acid encoding the chimeric protein of any of the herein disclosed aspects or embodiments. Ulcerative colitis (UC) is along-term condition that results in inflammation and ulcers of the colon and rectum. The primary symptom of active disease is abdominal pain and diarrhea mixed with blood. Weight loss, fever, and anemia may also occur. Often symptoms come on slowly and can range from mild to severe. Symptoms typically occur intermittently with periods of no symptoms between flares. Complications may include megacolon, inflammation of the eye, joints, or liver, and colon cancer. The cause of UC may involve immune system dysfunction, genetics, changes in the normal gut bacteria, and environmental factors. Several medications are used to treat symptoms including aminosalicylates such as sulfasalazine, steroids, immunosuppressants such as azathioprine, and biological therapy; which may be used in conjunction with the present disclosure.

In some embodiments, the subject has an Irritable Bowel Syndrome (IBS), including IBS with constipation (IBS-C); IBS with diarrhea (IBS-D), or IBS with mixed bowel habits (IBS-M).

In some embodiments, irritable bowel syndrome with constipation (IBS-C) manifests with symptoms including abdominal pain and discomfort, bloating, gas, and inability to pass stools. The cause of IBS-C is unknown, and there is no cure. Present treatments include lifestyle modifications, dietary changes, psychosocial therapy, and medications. In some embodiments, irritable bowel syndrome with diarrhea (IBS-D) manifests as IBS with increased diarrhea. The cause of IBS-D is unknown, and there is no cure. Present treatments include diet changes, stress relief, and over-the-counter anti-diarrheal medications. In some embodiments, irritable bowel syndrome with mixed bowel habits (IBS-M) manifests as IBS with alternating diarrhea and constipation symptoms. The cause of IBS-M is unknown, and there is no cure.

In embodiments, the chimeric protein used in the method of treating an autoimmune disease has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is alinker adjoining the first domain and the second domain. In this aspect, either or both of the first domain and the second domain decreases self-directed immune system activity when bound to its ligand/receptor.

In embodiments, the portion of the first domain is capable of binding the native ligand/receptor for the transmembrane protein, the secreted protein, or the membrane-anchored extracellular protein.

In embodiments, the portion of the second domain is capable of binding the native ligand/receptor for the transmembrane protein, the secreted protein, or the membrane-anchored extracellular protein.

In embodiments, the first domain comprises substantially the entire extracellular domain of the transmembrane protein, substantially the entire secreted protein, or substantially the entire membrane-anchored extracellular protein.

In embodiments, the second domain comprises substantially the entire extracellular domain of the transmembrane protein, substantially the entire secreted protein, or substantially the entire membrane-anchored extracellular protein.

In embodiments, the binding of the portion of the first domain to its ligand/receptor decreases immune system activity by activating an immune inhibitory signal or inhibiting an immune activating signal.

In embodiments, the binding of the portion of the second domain to its ligand/receptor decreases immune system activity by activating an immune inhibitory signal or by inhibiting an immune activating signal.

In embodiments, the portion of the first domain comprises a transmembrane protein, a secreted protein, or a membrane-anchored extracellular protein selected from TNFR2, IL11RA, DR3, MADCAM, VCAM, IL36R, IL18BP, DcR3, OSMR, gp130, IL23R, IL12RB1, ITGA4, and ITGB7.

In embodiments, the portion of the second domain comprises a transmembrane protein, a secreted protein, or a membrane-anchored extracellular protein selected from TGF-beta, DcR3, PD-L1, CCL20, CCL25, IL18BP, IL12A, IL27B, GITRL, and IL10.

In embodiments, the first domain comprises a portion of IL11RA and the second domain comprises a portion of DcR3.

In embodiments, the first domain comprises a portion of DR3 and the second domain comprises a portion of PD-L1.

In embodiments, the first domain comprises a portion of MADCAM and the second domain comprises a portion of CCL20.

In embodiments, the first domain comprises a portion of MADCAM and the second domain comprises a portion of CCL25.

In embodiments, the first domain comprises a portion of MADCAM and the second domain comprises a portion of PD-L1.

In embodiments, the first domain comprises a portion of VCAM and the second domain comprises a portion of PD-L1.

In embodiments, the first domain comprises a portion of IL36R and the second domain comprises a portion of DcR3.

In embodiments, the first domain comprises a portion of IL18BP and the second domain comprises a portion of DcR3.

In embodiments, the first domain comprises a portion of DcR3 and the second domain comprises a portion of IL18BP.

In embodiments, the first domain comprises a portion of OSMR and the second domain comprises a portion of DcR3.

In embodiments, the first domain comprises a portion of gp130 and the second domain comprises a portion of DcR3.

In embodiments, the first domain comprises a portion of DcR3 and the second domain comprises a portion of IL12A.

In embodiments, the first domain comprises a portion of DcR3 and the second domain comprises a portion of IL27B.

In embodiments, the first domain comprises a portion of IL23R and the second domain comprises a portion of DcR3.

In embodiments, the first domain comprises a portion of IL12RB1 and the second domain comprises a portion of DcR3.

In embodiments, the first domain comprises a portion of ITGA4 and the second domain comprises a portion of DcR3.

In embodiments, the first domain comprises a portion of ITGB7 and the second domain comprises a portion of DcR3.

In embodiments, the first domain comprises a portion of ITGA4 and the second domain comprises a portion of GITRL.

In embodiments, the first domain comprises a portion of ITGB7 and the second domain comprises a portion of GITRL.

In embodiments, the first domain comprises a portion of ITGA4 and the second domain comprises a portion of IL10.

In embodiments, the first domain comprises a portion of ITGB7 and the second domain comprises a portion of IL10.

In embodiments, the first domain comprises a portion of ITGA4 and the second domain comprises a portion of IL12A.

In embodiments, the first domain comprises a portion of ITGB7 and the second domain comprises a portion of IL27B.

In embodiments, the first domain comprises a portion of IL36R and the second domain comprises a portion of IL12A.

In embodiments, the first domain comprises a portion of IL36R and the second domain comprises a portion of IL27B.

In embodiments, the first domain comprises a portion of TNFR2 and the second domain comprises an extracellular domain of a transmembrane protein selected from TGF-beta, 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL; in embodiments, the second domain comprises an extracellular domain of TGF-beta. In embodiments, the CLEC family member is selected from AICL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+DC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D.

In embodiments, the binding of either or both of the first domain and the second domains to its ligand/receptor occurs with slow off rates (Koff), which provides a long interaction of a receptor and its ligand. In embodiments, the long interaction provides a prolonged decrease in immune system activity which comprises sustained activation of an immune inhibitory signal and/or a sustained inhibition of an immune activating signal. In embodiments, the sustained activation of the immune inhibitory signal and/or the sustained inhibition of the immune activating signal reduces the activity or proliferation of an immune cell, e.g., a B cell or a T cell. In embodiments, the sustained activation of the immune inhibitory signal and/or the sustained inhibition of the immune activating signal decreases synthesis and/or decreases release of a pro-inflammatory cytokine. In embodiments, the sustained activation of the immune inhibitory signal and/or the sustained inhibition of the immune activating signal increases synthesis and/or increases release of an anti-inflammatory cytokine. In embodiments, the sustained activation of the immune inhibitory signal and/or the sustained inhibition of the immune activating signal decreases antibody production and/or decreases secretion of antibodies by a B cell, e.g., an antibody that recognizes a self-antigen. In embodiments, the sustained activation of the immune inhibitory signal and/or the sustained inhibition of the immune activating signal decreases the activity of and/or decreases the number of T cytotoxic cells, e.g., which recognize a self-antigen and kill cells presenting or expressing the self-antigen. In embodiments, the sustained activation of the immune inhibitory signal and/or the sustained inhibition of the immune activating signal increases the activity and/or increases the number of T regulatory cells.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain, e.g., a hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the chimeric protein used in the method of treating an autoimmune disease comprises: (a) a first domain comprising a portion of IL11RA that is capable of binding a I111RA ligand, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL11RA-Fc-DcR3.

In embodiments, the chimeric protein used in the method of treating an autoimmune disease comprises: (a) a first domain comprising a portion of DR3 that is capable of binding a DR3 ligand/receptor, (b) a second domain comprising a portion of PD-L1 that is capable of binding PD-1, and (c) a linker linking the first domain and the second domain and comprising a hinge-CH2-CH3 Fc domain. In some embodiments, this chimeric protein is referred to herein as DR3-Fc-PD-L1.

In embodiments, the chimeric protein used in the method of treating an autoimmune disease comprises: (a) a first domain comprising a portion of MADCAM that is capable of binding a MADCAM ligand/receptor, (b) a second domain comprising a portion of CCL20 that is capable of binding a CCL20 receptor, and (c) a linker linking the first domain and the second domain and comprising a hinge-CH2-CH3 Fc domain. In some embodiments, this chimeric protein is referred to herein as MADCAM-Fc-CCL20.

In embodiments, the chimeric protein used in the method of treating an autoimmune disease comprises: (a) a first domain comprising a portion of MADCAM that is capable of binding a MADCAM ligand/receptor, (b) a second domain comprising a portion of CCL25 that is capable of binding a CCL25 receptor, and (c) a linker linking the first domain and the second domain and comprising a hinge-CH2-CH3 Fc domain. In some embodiments, this chimeric protein is referred to herein as MADCAM-Fc-CCL25.

In embodiments, the chimeric protein used in the method of treating an autoimmune disease comprises: (a) a first domain comprising a portion of MADCAM that is capable of binding a MADCAM ligand/receptor, (b) a second domain comprising a portion of PD-L1 that is capable of binding PD-1, and (c) a linker linking the first domain and the second domain and comprising a hinge-CH2-CH3 Fc domain. In some embodiments, this chimeric protein is referred to herein as MADCAM-Fc-PD-L1.

In embodiments, the chimeric protein used in the method of treating an autoimmune disease comprises: (a) a first domain comprising a portion of VCAM that is capable of binding a VCAM ligand/receptor, (b) a second domain comprising a portion of PD-L1 that is capable of binding PD-1, and (c) a linker linking the first domain and the second domain and comprising a hinge-CH2-CH3 Fc domain. In some embodiments, this chimeric protein is referred to herein as VCAM-Fc-PD-L1.

In embodiments, the chimeric protein used in the method of treating an autoimmune disease comprises: (a) a first domain comprising a portion of IL36R that is capable of binding an IL36R ligand, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL36R-Fc-DcR3.

In embodiments, the chimeric protein used in the method of treating an autoimmune disease comprises: (a) a first domain comprising a portion of IL18BP that is capable of binding a IL18BP ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL18BP-Fc-DcR3.

In embodiments, the chimeric protein used in the method of treating an autoimmune disease comprises: (a) a first domain comprising a portion of DcR3 that is capable of binding a DcR3 ligand, (b) a second domain comprising a portion of IL18BP that is capable of binding a IL18BP ligand/receptor, and (c) a linker linking the first domain and the second domain and comprising a hinge-CH2-CH3 Fc domain. In some embodiments, this chimeric protein is referred to herein as DcR3-Fc-IL18BP.

In embodiments, the chimeric protein used in the method of treating an autoimmune disease comprises: (a) a first domain comprising a portion of OSMR that is capable of binding an OSMR ligand, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as OSMR-Alpha-DcR3.

In embodiments, the chimeric protein used in the method of treating an autoimmune disease comprises: (a) a first domain comprising a portion of gp130 that is capable of binding a gp130 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as gp130-Beta-DcR3.

In embodiments, the chimeric protein used in the method of treating an autoimmune disease comprises: (a) a first domain comprising a portion of DcR3 that is capable of binding a DcR3 ligand, (b) a second domain comprising a portion of IL12A that is capable of binding a IL12A ligand/receptor, and (c) a linker linking the first domain and the second domain and comprising a hinge-CH2-CH3 Fc domain. In some embodiments, this chimeric protein is referred to herein as DcR3-Alpha-IL12A.

In embodiments, the chimeric protein used in the method of treating an autoimmune disease comprises: (a) a first domain comprising a portion of DcR3 that is capable of binding a DcR3 ligand, (b) a second domain comprising a portion of IL27B that is capable of binding a IL27B ligand/receptor, and (c) a linker linking the first domain and the second domain and comprising a hinge-CH2-CH3 Fc domain. In some embodiments, this chimeric protein is referred to herein as DcR3-Beta-IL27B.

In embodiments, the chimeric protein used in the method of treating an autoimmune disease comprises: (a) a first domain comprising a portion of IL23R that is capable of binding an IL23R ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL23R-Alpha-DcR3.

In embodiments, the chimeric protein used in the method of treating an autoimmune disease comprises: (a) a first domain comprising a portion of IL12RB1 that is capable of binding an IL12RB1 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as IL12RB1-Beta-DcR3.

In embodiments, the chimeric protein used in the method of treating an autoimmune disease comprises: (a) a first domain comprising a portion of ITGA4 that is capable of binding an ITGA4ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as ITGA4-Alpha-DcR3.

In embodiments, the chimeric protein used in the method of treating an autoimmune comprises: (a) a first domain comprising a portion of ITGB7 that is capable of binding an ITGB7 ligand/receptor, (b) a second domain comprising a portion of DcR3 that is capable of binding a DcR3 ligand, and (c) a linker linking the first domain and the second domain and comprising a hinge-CH2-CH3 Fc domain. In some embodiments, this chimeric protein is referred to herein as ITGB7-Beta-DcR3.

In embodiments, the chimeric protein used in the method of treating an autoimmune disease comprises: (a) a first domain comprising a portion of ITGA4 that is capable of binding an ITGA4ligand/receptor, (b) a second domain comprising a portion of GITRL that is capable of binding a GITRL ligand, and (c) a linker linking the first domain and the second domain and comprising a hinge-CH2-CH3 Fc domain. In some embodiments, this chimeric protein is referred to herein as ITGA4-Alpha-GITRL.

In embodiments, the chimeric protein used in the method of treating an autoimmune disease comprises: (a) a first domain comprising a portion of ITGB7 that is capable of binding an ITGB7 ligand/receptor, (b) a second domain comprising a portion of GITRL that is capable of binding a GITRL ligand, and (c) a linker linking the first domain and the second domain and comprising a hinge-CH2-CH3 Fc domain. In some embodiments, this chimeric protein is referred to herein as ITGB7-Beta-GITRL.

In embodiments, the chimeric protein used in the method of treating an autoimmune disease comprises: (a) a first domain comprising a portion of ITGA4 that is capable of binding an ITGA4ligand/receptor, (b) a second domain comprising a portion of IL10 that is capable of binding an IL10 receptor, and (c) a linker linking the first domain and the second domain and comprising a hinge-CH2-CH3 Fc domain. In some embodiments, this chimeric protein is referred to herein as ITGA4-Alpha-IL10.

In embodiments, the chimeric protein used in the method of treating an autoimmune disease comprises: (a) a first domain comprising a portion of ITGB7 that is capable of binding an ITGB7 ligand/receptor, (b) a second domain comprising a portion of IL10 that is capable of binding an IL10 receptor, and (c) a linker linking the first domain and the second domain and comprising a hinge-CH2-CH3 Fc domain. In some embodiments, this chimeric protein is referred to herein as ITGB7-Beta-IL10.

In embodiments, the chimeric protein used in the method of treating an autoimmune disease comprises: (a) a first domain comprising a portion of ITGA4 that is capable of binding an ITGA4ligand/receptor, (b) a second domain comprising a portion of IL12A that is capable of binding an IL12A ligand/receptor, and (c) a linker linking the first domain and the second domain and comprising a hinge-CH2-CH3 Fc domain. In some embodiments, this chimeric protein is referred to herein as ITGA4-Alpha-IL12A.

In embodiments, the chimeric protein used in the method of treating an autoimmune disease comprises: (a) a first domain comprising a portion of ITGB7 that is capable of binding an ITGB7 ligand/receptor, (b) a second domain comprising a portion of IL27B that is capable of binding an IL27B ligand/receptor, and (c) a linker linking the first domain and the second domain and comprising a hinge-CH2-CH3 Fc domain. In some embodiments, this chimeric protein is referred to herein as ITGB7-Beta-IL27B.

In embodiments, the chimeric protein used in the method of treating an autoimmune disease comprises: (a) a first domain comprising a portion of IL36R that is capable of binding an IL36R ligand, (b) a second domain comprising a portion of IL12A that is capable of binding an IL12A ligand/receptor, and (c) a linker linking the first domain and the second domain and comprising a hinge-CH2-CH3 Fc domain. In some embodiments, this chimeric protein is referred to herein as IL36R-Alpha-IL12A.

In embodiments, the chimeric protein used in the method of treating an autoimmune disease comprises: (a) a first domain comprising a portion of IL36R that is capable of binding an IL36R ligand, (b) a second domain comprising a portion of IL27B that is capable of binding an IL27B ligand/receptor, and (c) a linker linking the first domain and the second domain and comprising a hinge-CH2-CH3 Fc domain. In some embodiments, this chimeric protein is referred to herein as IL36R-Beta-IL27B.

In embodiments, the chimeric protein used in the method of treating an autoimmune disease comprises: (a) a first domain comprising a portion of TNFR2 that is capable of binding a TNFR2 ligand/receptor, (b) a second domain comprising a portion of TGF-beta that is capable of binding a TGF-beta ligand/receptor, and (c) a linker linking the first domain and the second domain and comprising a hinge-CH2-CH3 Fc domain. In some embodiments, this chimeric protein is referred to as TNFR2-Fc-TGF-beta.

In embodiments, the hinge-CH2-CH3 Fc domain comprises at least one cysteine residue capable of forming a disulfide bond. In embodiments, the hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the method further comprises administering to the subject an anti-inflammatory drug, e.g., a non-steroidal anti-inflammatory or a corticosteroid. In embodiments, the pharmaceutical composition and the anti-inflammatory drug are provided simultaneously (e.g., as two distinct pharmaceutical compositions or as a single pharmaceutical composition), the pharmaceutical composition is provided after the anti-inflammatory drug is provided, or the pharmaceutical composition is provided before the anti-inflammatory drug is provided. In embodiments, the non-steroidal anti-inflammatory is selected from the group consisting of acetyl salicylic acid (aspirin), benzyl-2,5-diacetoxybenzoic acid, celecoxib, diclofenac, etodolac, etofenamate, fulindac, glycol salicylate, ibuprofen, indomethacin, ketoprofen, methyl salicylate, nabumetone, naproxen, oxaprozin, phenylbutazone, piroxicam, salicylic acid, salicylmides, and vimovo® (a combination of naproxen and esomeprazole magnesium). In embodiments, the corticosteroid is selected from the group consisting of alpha-methyl dexamethasone, amcinafel, amcinafide, beclomethasone dipropionate, beclomethasone dipropionate., betamethasone and the balance of its esters, betamethasone benzoate, betamethasone dipropionate, betamethasone valerate, beta-methyl betamethasone, bethamethasone, chloroprednisone, clescinolone, clobetasol valerate, clocortelone, cortisone, cortodoxone, desonide, desoxymethasone, dexamethasone, dichlorisone, diflorasone diacetate, diflucortolone valerate, difluorosone diacetate, difluprednate, fluadrenolone, flucetonide, fluclorolone acetonide, flucloronide, flucortine butylester, fludrocortisone, flumethasone pivalate, flunisolide, fluocinonide, fluocortolone, fluoromethalone, fluosinolone acetonide, fluperolone, fluprednidene (fluprednylidene) acetate, fluprednisolone, fluradrenolone acetonide, flurandrenolone, halcinonide, hydrocortisone, hydrocortisone acetate, hydrocortisone butyrate, hydroxyltriamcinolone, medrysone, meprednisone, methylprednisolone, paramethasone, prednisolone, prednisone, triamcinolone, and triamcinolone acetonide.

In embodiments, the method further comprises administering to the subject an immunosuppressive agent. In embodiments, the pharmaceutical composition and the immunosuppressive agent are provided simultaneously (e.g., as two distinct pharmaceutical compositions or as a single pharmaceutical composition), the pharmaceutical composition is provided after the immunosuppressive agent is provided, or the pharmaceutical composition is provided before the immunosuppressive agent is provided. In embodiments, the immunosuppressive agent is selected from the group consisting of an antibody (e.g., basiliximab, daclizumab, and muromonab), an anti-immunophilin (e.g., cyclosporine, tacrolimus, and sirolimus), an antimetabolite (e.g., azathioprine and methotrexate), a cytostatic (such as alkylating agents), a cytotoxic antibiotic, an inteferon, a mycophenolate, an opioid, a small biological agent (e.g., fingolimod and myriocin), and a TNF binding protein.

In embodiments, the method further comprises administering to the subject an anti-inflammatory drug (as disclosed herein) and an immunosuppressive agent (as disclosed herein). The order of administration of the pharmaceutical composition comprising a therapeutically effective amount of the chimeric protein, the anti-inflammatory drug, and the immunosuppressive agent is not limited. As examples, the pharmaceutical composition may be administered before the anti-inflammatory drug and the immunosuppressive agent (e.g., which are formulated into a single pharmaceutical composition or as two pharmaceutical compositions); the pharmaceutical composition may be administered before the anti-inflammatory drug and after the immunosuppressive agent; the pharmaceutical composition may be administered with the anti-inflammatory drug (e.g., in a single pharmaceutical composition or in two pharmaceutical compositions) and before the immunosuppressive agent; the anti-inflammatory drug and the immunosuppressive agent may be administered in a single pharmaceutical composition or in two pharmaceutical compositions before the pharmaceutical composition is administered; and the pharmaceutical composition, the anti-inflammatory drug, and the immunosuppressive agent may be administered together, e.g., in a single composition.

In embodiments, the method treats an autoimmune disease selected from ankylosing spondylitis, diabetes mellitus, Grave's disease, Hashimoto's thyroiditis, hypersensitivity reactions (e.g., allergies, hay fever, asthma, and acute edema cause type I hypersensitivity reactions), inflammatory bowel diseases (e.g., colitis ulcerosa and Crohn's disease), multiple sclerosis, psoriasis, psoriasis, rheumatoid arthritis, sarcoidosis, Sjogren's syndrome, systemic lupus erythematosus, and vasculitis.

### Administration, Dosing, and Treatment Regimens

Routes of administration include, for example: intradermal, intratumoral, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

As examples, administration results in the release of chimeric protein (and/or an anti-inflammatory drug and/or an immunosuppressive agent) disclosed herein into the bloodstream (via enteral or parenteral administration), or alternatively, the chimeric protein (and/or an anti-inflammatory drug and/or an immunosuppressive agent) is administered directly to the site of active disease.

Any chimeric protein (and/or an anti-inflammatory drug and/or an immunosuppressive agent) disclosed herein can be administered orally. Any chimeric protein (and/or an anti-inflammatory drug and/or an immunosuppressive agent) can also be administered by any other convenient route, for example, by intravenous infusion or bolus injection, by absorption through epithelial or mucocutaneous linings (e.g., oral mucosa, rectal and intestinal mucosa, etc.). Administration can be systemic or local. Various delivery systems are known, e.g., encapsulation in liposomes, microparticles, microcapsules, or capsules, and can be used to facilitate administration.

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any chimeric protein (and/or an anti-inflammatory drug and/or an immunosuppressive agent) disclosed herein as well as the dosing schedule can depend on various parameters, including, but not limited to, the disease being treated, the subject's general health, and the administering physician's discretion.

Any chimeric protein disclosed herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an anti-inflammatory drug and/or an immunosuppressive agent, to a subject in need thereof.

In embodiments, a chimeric protein and an anti-inflammatory drug and/or an immunosuppressive agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart.

The dosage of any chimeric protein (and/or an anti-inflammatory drug and/or an immunosuppressive agent) disclosed herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

For administration of any chimeric protein disclosed herein by parenteral injection, the dosage may be about 0.1 mg to about 250 mg per day, about 1 mg to about 20 mg per day, or about 3 mg to about 5 mg per day. Generally, when orally or parenterally administered, the dosage of any chimeric protein disclosed herein may be about 0.1 mg to about 1500 mg per day, or about 0.5 mg to about 10 mg per day, or about 0.5 mg to about 5 mg per day, or about 200 to about 1,200 mg per day (e.g., about 200 mg, about 300 mg, about 400 mg, about 500 mg, about 600 mg, about 700 mg, about 800 mg, about 900 mg, about 1,000 mg, about 1,100 mg, about 1,200 mg per day).

In embodiments, administration of the chimeric protein disclosed herein is by parenteral injection at a dosage of about 0.1 mg to about 1500 mg per treatment, or about 0.5 mg to about 10 mg per treatment, or about 0.5 mg to about 5 mg per treatment, or about 200 to about 1,200 mg per treatment (e.g., about 200 mg, about 300 mg, about 400 mg, about 500 mg, about 600 mg, about 700 mg, about 800 mg, about 900 mg, about 1,000 mg, about 1,100 mg, about 1,200 mg per treatment).

In embodiments, a suitable dosage of the chimeric protein is in a range of about 0.01 mg/kg to about 100 mg/kg of body weight, or about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, inclusive of all values and ranges therebetween.

In embodiments, delivery of a chimeric protein disclosed herein (and/or an anti-inflammatory drug and/or an immunosuppressive agent) can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A chimeric protein (and/or an anti-inflammatory drug and/or an immunosuppressive agent) disclosed herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In embodiments, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any chimeric protein (and/or an anti-inflammatory drug and/or an immunosuppressive agent) disclosed herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any chimeric protein (and/or an anti-inflammatory drug and/or an immunosuppressive agent) disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any chimeric protein (and/or an anti-inflammatory drug and/or an immunosuppressive agent) disclosed herein can be administered in a single daily dose, or the total daily dosage can be administered in divided doses of two, three or four times daily. Furthermore, any chimeric protein (and/or an anti-inflammatory drug and/or an immunosuppressive agent) disclosed herein can be administered continuously rather than intermittently throughout the dosage regimen.

### Vectors and Cells

An aspect of the present disclosure is an expression vector comprising a nucleic acid encoding the chimeric protein of any of the herein disclosed aspects or embodiments. The expression vector comprises a nucleic acid encoding the chimeric protein disclosed herein. In embodiments, the expression vector comprises DNA or RNA. In embodiments, the expression vector is a mammalian expression vector.

An expression vector may be produced by cloning the nucleic acids encoding the three fragments (the first domain, followed by a linker sequence, followed by the second) into a vector (plasmid, viral or other). Accordingly, in embodiments, the present chimeric proteins are engineered as such.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and APL. Non-limiting examples of prokaryotic expression vectors may include the Agt vector series such as Agt11 (Huynh et a., in “DNA Cloning Techniques, Vol. I: A Practical Approach,” 1984, (D. Glover, ed.), pp. 49-78, IRL Press, Oxford), and the pET vector series (Studier et al., Methods Enzymol/1990, 185:60-89). Prokaryotic host-vector systems cannot perform much of the post-translational processing of mammalian cells, however. Thus, eukaryotic host-vector systems may be particularly useful. A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et a., Cancer Res 1989, 49:2735-42; and Taylor et a., Mol Cell Biol 1990, 10:165-75). Heat shock promoters or stress promoters also may be advantageous for driving expression of the chimeric proteins in recombinant host cells.

In embodiments, expression vectors of the invention comprise a nucleic acid encoding the chimeric proteins, or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the invention is capable of expressing operably linked encoding nucleic acid in a human cell. In embodiments, the cell is a tumor cell. In another embodiment, the cell is a non-tumor cell. In embodiments, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In embodiments, the present disclosure contemplates the use of inducible promoters capable of effecting high level of expression transiently in response to a cue. For example, when in the proximity of a tumor cell, a cell transformed with an expression vector for the chimeric protein (and/or an anti-inflammatory drug and/or an immunosuppressive agent) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (e.g., “upstream”). Expression control regions can also be located at the 3′ end of the transcribed sequence (e.g., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There is a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et a., J. Biol. Chem. 262, 4429-4432 (1987); and Wagner et a., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et a., TIG 15:326-332, 1999; Kootstra et a., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), FIp (Broach, et a!., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et a., supra), and components for integrating viruses such as AAV, retroviruses, and antiviruses having components that provide for virus integration such as the LTR sequences of retroviruses or lentivirus and the ITR sequences of AAV (Kootstra et a!., Ann. Rev. Pharm. Toxicol., 43:413-439, 2003). In addition, direct and targeted genetic integration strategies may be used to insert nucleic acid sequences encoding the chimeric fusion proteins including CRISPR/CAS9, zinc finger, TALEN, and meganuclease gene-editing technologies.

In embodiments, the expression vectors for the expression of the chimeric proteins (and/or an anti-inflammatory drug and/or an immunosuppressive agent) are viral vectors. Many viral vectors useful for gene therapy are known (see, e.g., Lundstrom, Trends Biotechnol., 21: 1 17, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In embodiments, the invention provides methods of transducing a human cell in vivo, comprising contacting a solid tumor in vivo with a viral vector of the invention.

Another aspect of the present disclosure is a host cell comprising the expression vector of the preceding aspect and embodiments.

Expression vectors can be introduced into host cells for producing the present chimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et a., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the chimeric proteins disclosed herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, cancer patients, and patients with an infectious disease, private laboratory deposits, public culture collections such as the American Type Culture Collection (ATCC), or from commercial suppliers.

Cells that can be used for production of the present chimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, and fetal liver. The choice of cell type depends on the type of tumor or infectious disease being treated or prevented, and can be determined by one of skill in the art.

Production and purification of Fc-containing macromolecules (such as monoclonal antibodies) has become a standardized process, with minor modifications between products. For example, many Fc containing macromolecules are produced by human embryonic kidney (HEK) cells (or variants thereof) or Chinese Hamster Ovary (CHO) cells (or variants thereof) or in some cases by bacterial or synthetic methods. Following production, the Fc containing macromolecules that are secreted by HEK or CHO cells are purified through binding to Protein A columns and subsequently ‘polished’ using various methods. Generally speaking, purified Fc containing macromolecules are stored in liquid form for some period of time, frozen for extended periods of time or in some cases lyophilized. In embodiments, production of the chimeric proteins contemplated herein may have unique characteristics as compared to traditional Fc containing macromolecules. In certain examples, the chimeric proteins may be purified using specific chromatography resins, or using chromatography methods that do not depend upon Protein A capture. In embodiments, the chimeric proteins may be purified in an oligomeric state, or in multiple oligomeric states, and enriched for a specific oligomeric state using specific methods. Without being bound by theory, these methods could include treatment with specific buffers including specified salt concentrations, pH and additive compositions. In other examples, such methods could include treatments that favor one oligomeric state over another. The chimeric proteins obtained herein may be additionally ‘polished’ using methods that are specified in the art. In embodiments, the chimeric proteins are highly stable and able to tolerate a wide range of pH exposure (between pH 3-12), are able to tolerate a large number of freeze/thaw stresses (greater than 3 freeze/thaw cycles) and are able to tolerate extended incubation at high temperatures (longer than 2 weeks at 40 degrees C.). In embodiments, the chimeric proteins are shown to remain intact, without evidence of degradation, deamidation, etc. under such stress conditions.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish. In embodiments, the subject and/or animal may comprise fluorescently-tagged cells (with e.g., GFP). In embodiments, the subject and/or animal is a transgenic animal comprising a fluorescent cell.

In embodiments, the subject and/or animal is a human. In embodiments, the human is a pediatric human. In embodiments, the human is an adult human. In embodiments, the human is a geriatric human. In embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In embodiments, the subject is a non-human animal, and therefore the invention pertains to veterinary use.

In a specific embodiment, the non-human animal is a household pet. In another specific embodiment, the non-human animal is a livestock animal.

### Kits and Medicaments

Aspects of the present disclosure provide kits that can simplify the administration of any chimeric protein or pharmaceutical composition as disclosed herein.

An illustrative kit of the invention comprises any chimeric protein and/or pharmaceutical composition disclosed herein in unit dosage form. In embodiments, the unit dosage form is a container, such as a pre-filled syringe, which can be sterile, containing any agent disclosed herein and a pharmaceutically acceptable carrier, diluent, excipient, or vehicle. The kit can further comprise a label or printed instructions instructing the use of any agent disclosed herein. The kit may also include a lid speculum, topical anesthetic, and a cleaning agent for the administration location. The kit can also further comprise one or more additional agent disclosed herein. In embodiments, the kit comprises a container containing an effective amount of a composition of the invention and an effective amount of another composition, such those disclosed herein.

The chimeric protein of any of the herein disclosed aspects or embodiments may be used as a medicament in the treatment of an autoimmune disease, e.g., selected from ankylosing spondylitis, diabetes mellitus, Grave's disease, Hashimoto's thyroiditis, hypersensitivity reactions (e.g., allergies, hay fever, asthma, and acute edema cause type I hypersensitivity reactions), inflammatory bowel diseases (e.g., colitis ulcerosa and Crohn's disease), multiple sclerosis, psoriasis, psoriasis, rheumatoid arthritis, sarcoidosis, Sjogren's syndrome, systemic lupus erythematosus, and vasculitis.

The present disclosure includes the use of the chimeric protein of any of the herein-disclosed aspects or embodiments in the manufacture of a medicament.

Any aspect or embodiment disclosed herein can be combined with any other aspect or embodiment as disclosed herein.

The invention will be further described in the following examples, which do not limit the scope of the invention described in the claims.

### EXAMPLES

### Example 1. In Vivo Characterization of Chimeric Proteins in IBS Model

A DSS-induced mouse model of irritable bowel syndrome (IBS) was used to assess the efficacy of various chimeric proteins in treating IBS, particularly by evaluating which chimeric proteins protected mice from weight loss.

In particular, mice were divided into the following groups:

| | | | |
|---|---|---|---|
| No DSS | Group 1 Cage 1 | Chimeric Protein A | Group 4 Cage 7 |
| | Group 1 Cage 2 | 0.9 mg/ml | Group 4 Cage 8 |
| DSS only | Group 2 Cage 3 | mTNFR2-Fc- | Group 5 Cage 9 |
| | Group 2 Cage 4 | TGF-beta | Group 5 Cage 10 |
| | | 2.4 mg/ml | |
| mCTLA4-Ig | Group 3 Cage 5 | Chimeric Protein B | Group 6 Cage 11 |
| BioXcell | Group 3 Cage 6 | 7 mg/ml | Group 6 Cage 12 |
| #BE0099 | | | |

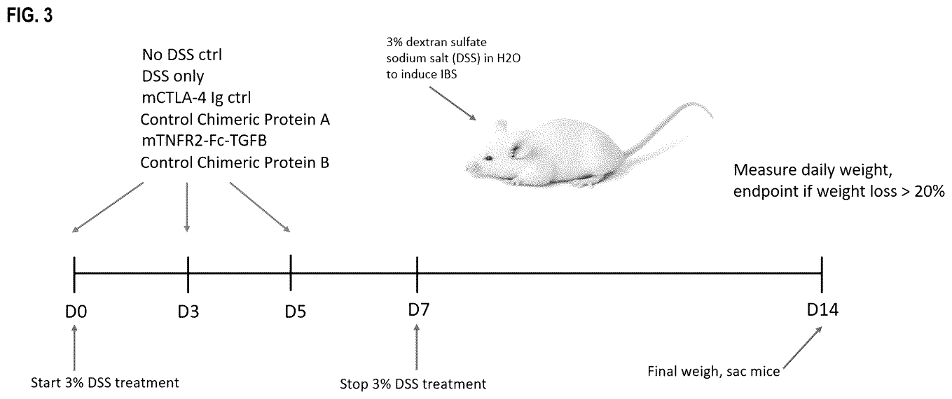

FIG. 3 depicts an overall schematic of the experiment. Specifically, mice were administered 3% DSS treatment at Day 0, and the DSS treatment was terminated on Day 7. Concurrently, the mice were administered the following (according to the group division above) on Days 0, 3, and 5: (1) No DSS (control); (2) DSS only; (3) mCTLA-4 Ig (control); (4) control chimeric protein A; (5) mTNFR2-Fc-TGF-beta chimeric protein; and (6) control chimeric protein B. The mice were weighed daily, with an endpoint if the weight loss was greater than 20%. On Day 14, the mice were weighed for a final time and sacrifice.

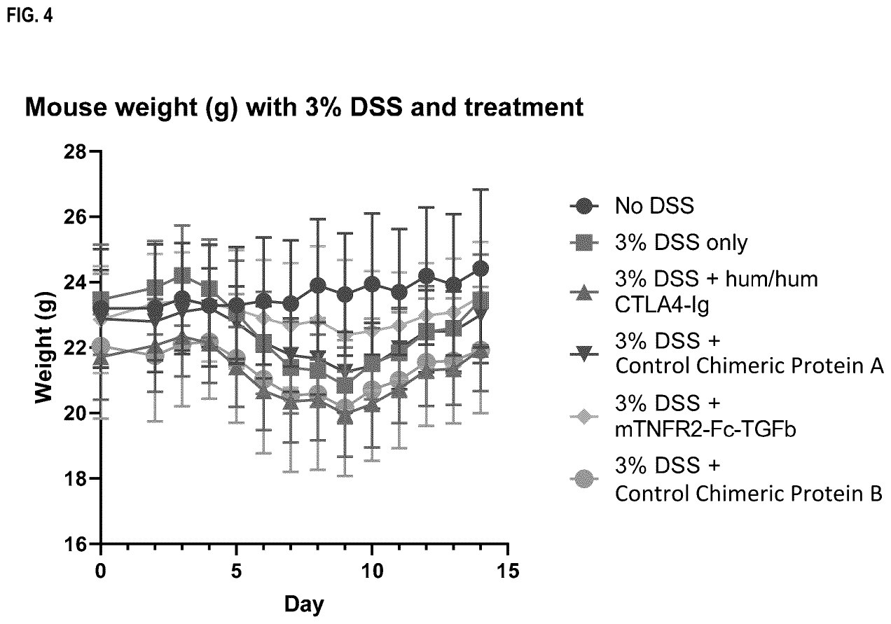

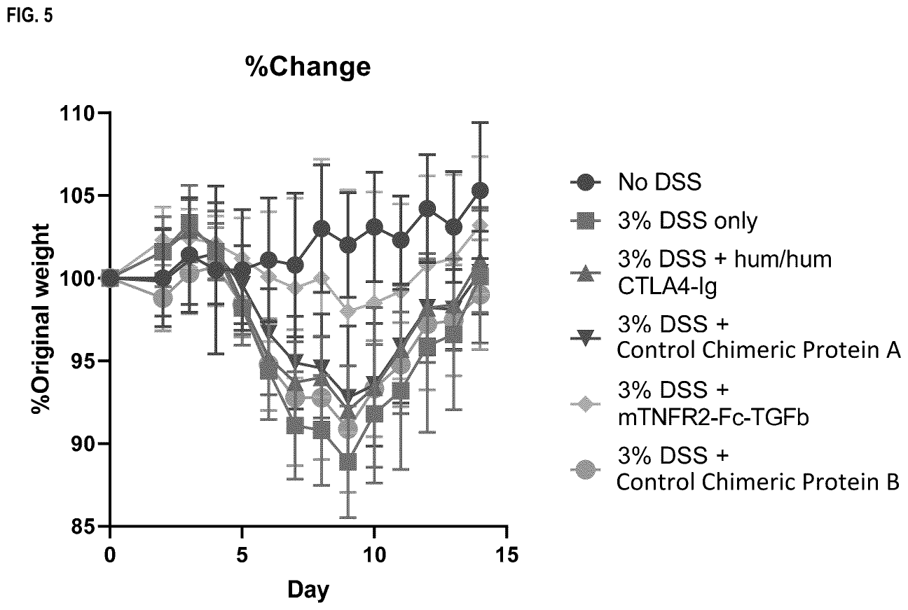

FIG. 4 shows mouse weight (g) with 3% DSS and various treatments over the course of the two-week experiment. The results shown in FIG. 4 demonstrate that, among the chimeric protein treatments, the group that was administered mTNFR2-Fc-TGF-beta (mTNFR2-Fc-TGF-beta) exhibited the greatest protection from weight loss. This finding is confirmed when the data is presented in terms of percent change. FIG. 5 shows that the mice administered with the mTNFR2-Fc-TGF-beta chimeric protein suffered the least from a percent change from their original weight.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

1.-125. (canceled)

126. A method of treating an autoimmune disease in a subject in need thereof, the method comprising administering to the subject in need thereof a pharmaceutical composition comprising a nucleic acid encoding a chimeric protein, wherein the chimeric protein comprises:
(a) a first domain comprising an extracellular domain of TNFR2 that is capable of binding an TNFR2 ligand,
(b) a second domain comprising a portion of TGF-beta that is capable of binding a TGF-beta receptor, and
(c) a linker linking the first domain and the second domain and comprising a hinge-CH2-CH3 Fc domain.

127. The method of claim 126, wherein the extracellular domain of TNFR2 comprises an amino acid sequence that is at least 95% identical to the amino acid sequence of SEQ ID NO: 102.

128. The method of claim 126, wherein the portion of TGF-beta comprises an amino acid sequence that is at least 95% identical to the amino acid sequence of SEQ ID NO: 103.

129. The method of claim 126, wherein the hinge-CH2-CH3 Fc domain is derived from IgG1 or IgG4.

130. The method of claim 129, wherein the IgG4 is a human IgG4.

131. The method of claim 126, wherein the linker comprises an amino acid sequence that is at least 95% identical to the amino acid sequence of SEQ ID NO: 1, SEQ ID NO: 2, or SEQ ID NO: 3.

132. The method of claim 131, wherein the linker further comprises one or more joining linkers having an amino acid sequences independently selected from SEQ ID NO: 4 to SEQ ID NO: 50.

133. The method of claim 132, wherein the linker comprises two or more joining linkers, wherein one joining linker is N terminal to the hinge-CH2-CH3 Fc domain and another joining linker is C terminal to the hinge-CH2-CH3 Fc domain

134. The method of claim 126, wherein the chimeric protein comprises an amino acid sequence that is at least 95% identical to the amino acid sequence of SEQ ID NO: 104.

135. The method of claim 126, wherein the administration causes a decrease in immune system activity which comprises sustained activation of an immune inhibitory signal and/or a sustained inhibition of an immune activating signal.

136. The method of claim 135, wherein the decrease in immune system activity comprises sustained activation of an immune inhibitory signal and/or a sustained inhibition of an immune activating signal.

137. The method of claim 126, wherein the nucleic acid is or comprises an mRNA, modified mRNA (mmRNA) or DNA.

138. The method of claim 137, wherein the nucleic acid is or comprises an mmRNA that comprises one or more nucleoside modifications.

139. The method of claim 138, wherein the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

140. The method of claim 139, wherein the mmRNA further comprises a 5′-cap and/or a poly A tail.

141. The method of claim 126, wherein the pharmaceutical composition is formulated as a lipid nanoparticle (LNP), a lipoplex, or a liposome.

142. The method of claim 141, wherein the pharmaceutical composition is formulated as a lipid nanoparticle (LNP).

143. The method of claim 142, wherein the lipid nanoparticles comprise lipids selected from an ionizable lipid, a structural lipid, cholesterol, and a polyethyleneglycol (PEG)-lipid

144. The method of claim 143, wherein the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle.

145. The method of claim 126, wherein the autoimmune disease is irritable bowel syndrome, inflammatory bowel disease, Crohn's disease or ulcerative colitis.
